(19) United States
(12) Patent Application Publication
(10) Pub. No.: US 2025/0051367 A1
(43) Pub. Date: Feb. 13, 2025

(54) HETEROCYCLE-CONTAINING LONP1 INHIBITOR COMPOUNDS, USES AND METHODS

(71) Applicant: Pretzel Therapeutics, Inc., Waltham, MA (US)

(72) Inventors: Jeremy Green, Waltham, MA (US); Paul Charifson, Carrabassett Valley, ME (US)

(73) Assignee: Pretzel Therapeutics, Inc., Waltham, MA (US)

(21) Appl. No.: 18/716,914

(22) PCT Filed: Dec. 6, 2022
(86) PCT No.: PCT/US2022/052008

§ 371 Date: Jun. 5, 2024

Related U.S. Application Data
(60) Provisional application No. 63/286,441, filed on Dec. 6, 2021.
(60) Provisional application No. 63/290,841, filed on Dec. 17, 2021.
(60) Provisional application No. 63/319,596, filed on Mar. 14, 2022.
(60) Provisional application No. 63/358,741, filed on Jul. 6, 2022.

(51) Int. Cl. C07F 5/02; A61K 31/69

(52) U.S. Cl. CPC C07F 5/025; A61K 31/69

(57) ABSTRACT

Disclosed are compounds according to Formula (1) which inhibit LONP1, and pharmaceutical compositions comprising compounds of the disclosure. Compounds and pharmaceutical compositions of the disclosure may be useful for the treatment of diseases and disorders associated with LONP1, including oncologic diseases and disorders, such as cancer, and diseases and disorders related to mitochondrial dysfunction, such as neurodegenerative disorders, metabolic disorders, and diseases associated with the aging process. The disclosure also relates to methods of using such compounds and compositions for the treatment of such diseases and disorders.

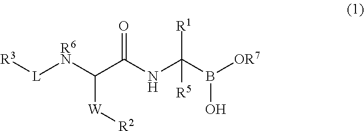

### FIELD OF THE INVENTION

The present invention relates to novel LONP1 inhibitors, their pharmaceutically acceptable salts, and pharmaceutical compositions thereof. The present invention also relates to methods of using such compounds and compositions, including to inhibit LONP1 and to treat oncologic diseases and disorders, such as cancer, and various diseases and disorders related to mitochondrial dysfunction, such as neurodegenerative disorders, metabolic disorders, and diseases associated with the aging process.

### BACKGROUND OF THE INVENTION

The mitochondrial Lon serine protease, LONP1, is an enzyme that is a member of the AAA+ superfamily of proteases (i.e., ATP-dependent proteases (ATPases) associated with diverse cellular activities). Widely conserved across eukaryotic species, human LONP1 is a 959-amino acid protein that consists of three domains: the N-terminal domain involved in substrate binding, the AAA+ (ATPase) domain, and the C-terminal domain (named the P-domain) involved in proteolytic activity. The ATPase and protease domains are the most well-conserved across species, while the N-terminal domain is the most variable.

LONP1 performs at least four different functions: proteolysis of damaged and oxidized proteins of the mitochondrial matrix; chaperone activity, namely the correct folding of proteins imported into the mitochondria; regulation of mitochondrial protein levels, including mitochondrial transcription factor A (TFAM); and binding to mitochondrial DNA (“mtDNA”) and RNA. As for the proteolytic activity of LONP1, like all the other proteases in the AAA+ family, it binds its substrate, unfolds it using the ATPase domain, and then digests it from the N or C-terminus. Its chaperone activity, mediated by the ATP-binding domain and the N-terminal domain, is crucial for mitochondrial homeostasis, as it is involved in the assembly of mitochondrial membrane complexes.

LONP1 has multiple, natural substrates, one of which is the mtDNA binding and packaging protein TFAM. TFAM has a crucial role in transcription initiation and mtDNA replication. Inhibition of LONP1 reportedly leads to increased levels of the TFAM protein, which in turn may lead to higher levels of mtDNA. TFAM and mtDNA have a mutual dependence for stability, whereby TFAM binds mtDNA and protects it from degradation, but when not bound to mtDNA, TFAM is rapidly degraded. LONP1 has been shown to regulate mtDNA copy number in Drosophila melanogaster by cleaving TFAM. In human cells with severe mtDNA deficits, depletion of LONP1 can increase levels of TFAM and upregulate mtDNA content.

Another natural substrate of LONP1 is POLγA, the catalytic subunit of DNA polymerase γ (POLγ). POLγ is the main protein responsible for mitochondrial DNA (mtDNA) replication. The accessory POLγB subunit acts to stabilize POLγA and to prevent LONP1-dependent degradation. Disease causing mutations such as A467T weaken interactions between POLγA and POLγB, which in turn makes POLγA susceptible to degradation by LONP1.

LONP1 is also required during embryogenesis. A homozygous deletion of the LONP1 gene in a mouse causes embryonic lethality. In line with this observation, mutations that change LONP1 activity during embryogenesis can cause a congenital syndrome known as CODAS, characterized by Cerebral, Ocular, Dental, Auricular and Skeletal anomalies. Further supporting a role during embryogenesis, defective mitochondrial protease LONP1 has also been linked to a classical, congenital mitochondrial disease. The mutant (Tyr565His) protein displayed higher ATPase activity, but reduced protease activity (see Peter, B. et. al., “Defective Mitochondrial Protease LonP1 Can Cause Classical Mitochondrial Disease,” Hum. Mol. Genet., 27, 10, 1743-1750 (2018)).

Additionally, LONP1 has a central role in the regulation of mitochondrial function, impacting bioenergetics in various cells and often causing disease (see Gibellini L. et. al., “LonP1 Differently Modulates Mitochondrial Function and Bioenergetics of Primary Versus Metastatic Colon Cancer Cells,” Front. Oncol. 8, 254 (2018). LONP1 upregulation is a characteristic shared by various types of cancer cells. Higher expression of LONP1 is correlated with tumor progression and aggressiveness. For instance, LONP1 overproduction is functionally linked to colorectal cancer cells by inducing the epithelial mesenchymal transition, an early step in the formation of metastases (see id). Furthermore, LONP1 is a regulator of mitochondrial proteostasis, which is required for maintaining the respiratory chain and degrading misfolded, oxidatively damaged or unassembled proteins. As such, inhibition of LONP1 is believed to be a mechanism by which various oncogenic diseases, such as cancers may be treated.

Similarly, multiple myeloma is an exceedingly prevalent and incurable cancer in the elderly (see Maneix, L. et al., “The Mitochondrial Protease LonP1 Promotes Proteasome Inhibitor Resistance in Multiple Myeloma,” Cancers 13, 843, 14-19 (2021)). Proteasome inhibitors are a common treatment for myeloma, but for unknown reasons, over time, a resistance to treatment develops. Compounds that inhibit LONP1 may provide a means to more thoroughly understand the molecular mechanisms that lead to such drug resistance in the treatment of multiple myeloma (see id).

While aspects of LONP1 biochemistry are known, its full physiological role in mitochondrial gene expression and homeostasis, as well as its underlying impact in the etiology of various disease states, remains unclear. LONP1 inhibitors will provide insight into, for example, the relationship between LONP1, mtDNA copy number, and human diseases. Pharmacological inhibition of LONP1 is one means by which to gain a further understanding of the role of this protease in cell physiology and the development of disease. LONP1 inhibitors have been reported, for example, in Kingsley, L. J. et al., J. Med. Chem. 64, 8, 4857-4869 (2021). In view of the numerous and varied roles of LONP1, there is a need for additional, potent, and specific inhibitors of LONP1.

### SUMMARY OF THE INVENTION

Provided are compounds, pharmaceutically acceptable salts of the compounds, pharmaceutical compositions comprising the compounds or their salts, methods of using the compounds, salts of the compounds, or pharmaceutical compositions of the compounds or their salts, and therapeutic uses of the compounds, or pharmaceutical compositions of the compounds or their salts, for treating diseases related to oncologic diseases and disorders, such as cancer, and/or various diseases and disorders related to mitochondrial dysfunction, such as neurodegenerative disorders, metabolic disorders, and diseases associated with the aging process. The compounds and their pharmaceutically acceptable salts are particularly useful as inhibitors of LONP1.

In one aspect there is provided a compound of structural Formula 1:

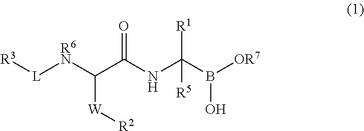

or a pharmaceutically acceptable salt, solvate, stereoisomer or mixture of stereoisomers, tautomer, isotopic form, pharmaceutically active metabolite thereof, or combinations thereof, wherein:
R¹ is selected from the group consisting of: deuterium, C1-C4 alkyl, C1-C4 alkoxyl, C1-C4 oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R⁸, CONR⁸R⁹, NR⁸R⁹, SR⁸, SO2NR⁸R⁹, C1-C4 alkyl, C1-C4 haloalkyl, C1-C4 alkoxy, phenyl, or a 5- or 6-membered heteroaryl;
W is C1-C4 alkyl, optionally substituted with one or more of deuterium, halogen, hydroxyl, CN, methyl or ethyl;
R² is a 5 to 14 membered heterocyclic mono-, bi- or tricyclic ring optionally having one or more heteroatoms selected from N, O and S, wherein the heterocyclic ring is optionally substituted with one or more substituent selected from deuterium, halogen, hydroxyl, CN, C1-C4 alkyl, C1-C4 haloalkyl or C1-C4 alkoxy;
L is C(O), C(O)O, C(O)NR⁴, S(O)₂, or a bond;
R³ is C₁-C₄ alkyl optionally substituted with one or more substituents each independently selected from the group consisting of deuterium, halogen, cyano, hydroxyl, C₁-C₄ alkoxyl, 5 or 6 membered aryl (e.g. phenyl) or 5 or 6 membered heteroaryl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C₁-C₄ alkoxyl, or C₁-C₄ alkyl that is optionally substituted with one to three substituents selected from deuterium, halogen, cyano, hydroxyl, or C₁-C₄ alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R⁸, CONR⁸R⁹, NR⁸R⁹, SR⁸, SO2NR⁸R⁹, C₁-C₄ alkoxyl, or C₁-C₄ alkyl that is optionally substituted with one to three substituents selected from deuterium, halogen, cyano, hydroxyl, or C₁-C₄ alkoxyl;
R⁴ is hydrogen, deuterium, or C1-C4 alkyl optionally substituted with one or more of halogen, hydroxyl and phenyl, wherein phenyl is optionally substituted with one or more substituent selected from halogen, hydroxyl and C1-C2 alkyl;
R⁵ is selected from hydrogen, deuterium or C1-C2 alkyl;
R⁶ is selected from hydrogen, deuterium or C1-C2 alkyl optionally substituted with one or more substituents each independently selected from the group consisting of halogen, hydroxyl, cyano, methoxyl and phenyl;
R⁷ is hydrogen, or R⁷ and R¹, together with the boron atom to which —OR⁷ is attached form a 5-membered heteroalkyl ring; and
R⁸ and R⁹ are each independently selected from hydrogen, deuterium, C1-C4 alkyl; C1-C4 haloalkyl, C1-C5 alkyl-alkoxyl, C3-C7 cycloalkyl, or R⁸ and R⁹ together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

Embodiments of the present disclosure include compounds of the disclosure (that is, compounds of Formula 1) or their pharmaceutically acceptable salts wherein one or more hydrogen atom is substituted with a deuterium atom.

Another aspect of the disclosure is directed to pharmaceutical compositions comprising a compound of the disclosure (that is, compounds of Formula 1) or a pharmaceutically acceptable salt thereof and a pharmaceutically acceptable excipient.

Other aspects of the disclosure are directed to methods of treating a disease or disorder, such as a disease or disorder characterized by mitochondrial dysfunction, such methods comprising administering to a subject in need thereof a therapeutically effective amount of a compound of the disclosure, a pharmaceutically acceptable salt thereof, or a composition comprising such as compound.

In various aspects and embodiments of the methods and therapeutic uses disclosed herein, the disease is selected from Alper's syndrome (Alpers-Huttenlocher syndrome), ataxia neuropathy syndrome (ANS), Mitochondrial DNA Depletion Syndrome (MDDS), Leigh Syndrome (Leigh Disease), Leber's Hereditary Optic Neuropathy (LHON), chronic progressive external ophthalmoplegia (CPEO), myoclonic epilepsy myopathy sensory ataxia (MEMSA), MELAS (Mitochondrial Encephalopathy, Lactic Acidosis, and Stroke-like episodes) syndrome, MERRF (myoclonus epilepsy with ragged-red fibers) syndrome, mitochondrial neurogastrointestinal encephalomyopathy (MNGIE), neuropathy, ataxia, and retinitis pigmentosa (NARP), Kearn's-Sayre Syndrome (KSS), and Pearson's Syndrome. In some aspects and embodiments the disease or disorder is selected from Alzheimer's disease, Parkinson's disease, obesity, diabetes, non-alcoholic steatohepatitis (NASH), and related metabolic syndromes such as non-alcoholic fatty liver disease (NAFLD).

Other aspects of the disclosure are directed to compounds or (pharmaceutical) compositions comprising compounds of the disclosure for use in methods for treating a disease or disorder, such as a disease or disorder characterized by mitochondrial dysfunction. These therapeutic uses may comprise administering to a subject in need thereof a therapeutically effective amount of a compound of the disclosure, a pharmaceutically acceptable salt thereof, or a composition comprising such a compound. Suitable diseases or disorders are those described above and herein below.

In some embodiments, the disease to be treated with a compound or composition of the disclosure is associated with mtDNA mutations or deletions, for example: m.3243A>G, m.11778G>A, m.14484T>C, m.3460G>A, m.8344A>G, m.3271T>C, m.3251A>G, m.8356T>C, m.4274T>C, m.14709T>C, m.12320A>G, m.4269A>G, m.12258C>A, m.1606G>A, m.10010T>C, m.7445A>G and m.1555A>G (see https://mitomap.org/MITOMAP).

Additional aspects and embodiments of the disclosure relate to methods of treating cancers and compounds or compositions for use in such methods: for example, those identified in Wong, K. S. et al. “Recent Advances in Targeting Human Mitochondrial AAA+ Proteases to Develop Novel Cancer Therapeutics,” Advances in Experimental Medicine and Biology, 1158,119-142 (2019), wherein the use or method comprising using a compound or composition of the disclosure or its pharmaceutically acceptable salt.

Further aspects and embodiments of the disclosure relate to methods of treating cancer, neurodegenerative disorders, metabolic disorders, and diseases associated with the aging process; and compounds and compositions of the disclosure for use in such methods.

Within the scope of this disclosure it is expressly intended that the various aspects, embodiments, examples and alternatives set out in the preceding paragraphs, in the claims and/or in the following description, and in particular the individual features thereof, may be taken independently or in any combination. That is, all embodiments and/or features of any aspect or embodiment can be combined in any way and/or combination, unless such features are incompatible. More particularly, it is specifically intended that any embodiment of any aspect may form an embodiment of any other aspect, and all such combinations are encompassed within the scope of the disclosure. The applicant reserves the right to change any originally filed claim or file any new claim accordingly, including the right to amend any originally filed claim to depend from and/or incorporate any feature of any other claim although not originally claimed in that manner.

### DETAILED DESCRIPTION OF THE INVENTION

Described herein are compounds and compositions (e.g. organic molecules, research tools, pharmaceutical formulations and therapeutics); uses for the compounds and compositions of the disclosure (in vitro and in vivo); as well as corresponding methods, whether diagnostic, therapeutic or for research applications. The chemical synthesis and biological testing of the compounds of the disclosure are also described. Beneficially, the compounds, compositions, uses and methods have utility in research towards and/or the treatment of diseases or disorders in animals, such as humans. Diseases or disorders which may benefit from LONP1 modulation include mitochondrial diseases, cancer and/or oncologic disease.

However, the compounds of the disclosure may also or alternatively be useful as lead molecules for the selection, screening and development of further derivatives that may have one or more improved beneficial drug property, as desired.

The disclosure also encompasses salts, solvates and functional derivatives of the compounds described herein. These compounds may be useful in the treatment of diseases or disorders characterized by mitochondrial disfunction; particularly those which may benefit from LONP1 inhibition.

Inhibitors of LONP1 are useful in compositions and methods suitable for treating many disorders, such as disorders characterized by mitochondrial dysfunction, including cancer. In some embodiments, the disease is selected from the group consisting of adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma colon cancer, colorectal cancer, cancer of connective tissue, esophageal cancer, embryonal carcinoma, fibrosarcoma, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas, mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, and vulvar cancer.

### Definitions

Unless defined otherwise, all technical and scientific terms used herein have the same meaning as commonly understood by one of ordinary skill in the art (e.g. in organic, physical or theoretical chemistry; biochemistry and molecular biology).

Unless otherwise indicated, the practice of the present disclosure employs conventional techniques in chemistry and chemical methods, biochemistry, molecular biology, pharmaceutical formulation, and delivery and treatment regimens for patients, which are within the capabilities of a person of ordinary skill in the art. Such techniques are also described in the literature cited herein. All documents cited in this disclosure are herein incorporated by reference in their entirety. Prior to setting forth the further detailed description and Examples of this disclosure, a number of definitions are provided that will assist in the understanding of the disclosure.

In accordance with this disclosure, the terms ‘molecule’ or ‘molecules’ are used interchangeably with the terms ‘compound’ or ‘compounds’, and sometimes the term ‘chemical structure’. The term ‘drug’ is typically used in the context of a pharmaceutical, pharmaceutical composition, medicament or the like, which has a known or predicted physiological or in vitro activity of medical significance; but such characteristics and qualities are not excluded in a molecule or compound of the disclosure. The term ‘drug’ is therefore used interchangeably with the alternative terms and phrases ‘therapeutic (agent)’, ‘pharmaceutical (agent)’, and ‘active (agent)’. Therapeutics according to the disclosure also encompass compositions and pharmaceutical formulations comprising the compounds of the disclosure.

Prodrugs and solvates of the compounds of the disclosure are also encompassed within the scope of the disclosure. The term ‘prodrug’ means a compound (e.g. a drug precursor) that is transformed in vivo to yield a compound of the disclosure or a pharmaceutically acceptable salt, solvate or ester of the compound. The transformation may occur by various mechanisms (e.g. by metabolic or chemical processes), such as by hydrolysis of a hydrolysable bond, e.g. in blood (see Higuchi & Stella (1987), “Pro-drugs as Novel Delivery Systems”, vol. 14 of the A.C.S. Symposium Series; (1987), “Bioreversible Carriers in Drug Design”, Roche, ed., American Pharmaceutical Association and Pergamon Press). The compositions and medicaments of the disclosure therefore may comprise prodrugs of the compounds of the disclosure. In some aspects and embodiments the compounds of the disclosure may be themselves prodrugs which may be metabolized in vivo to give the therapeutically effective compound.

The scope of this disclosure also includes various deuterated forms of the compounds of any of Formula 1 (inc. corresponding subgeneric formulas defined herein), respectively, or a pharmaceutically acceptable salt and/or a corresponding tautomer form thereof (including subgeneric formulas, as defined above). Each available hydrogen atom attached to a carbon atom may be independently replaced with a deuterium atom. A person of ordinary skill in the art will know how to synthesise deuterated forms of the compounds of Formula 1 disclosed herein (including subgeneric formulas, as defined above) or a pharmaceutically acceptable salt and/or a corresponding tautomer form thereof (including subgeneric formulas, as defined herein) of the present disclosure. For example, deuterated materials, such as alkyl groups may be prepared by conventional techniques (see for example: methyl-d3-amine available from Aldrich Chemical Co., Milwaukee, WI, Cat. No. 489,689-2).

The disclosure also includes isotopically-labelled compounds which are identical to those recited in Formula 1 disclosed herein (inc. corresponding subgeneric formulas defined herein), respectively, or a pharmaceutically acceptable salt and/or a corresponding tautomer form thereof (including subgeneric formulas, as defined above), but for the fact that one or more atoms are replaced by an atom having an atomic mass or mass number different from the atomic mass or mass number most commonly found in nature. Examples of isotopes that can be incorporated into compounds of this disclosure include isotopes of hydrogen, carbon, nitrogen, oxygen, fluorine, iodine and chlorine such as 3H, 11 C, 14 C, 18 F, 123 I or 125 I. Compounds of the present disclosure and pharmaceutically acceptable salts of said compounds that contain the aforementioned isotopes and/or other isotopes of other atoms are within the scope of this disclosure. Isotopically labelled compounds of the present disclosure, for example those into which radioactive isotopes such as 3H or 14 C have been incorporated, are useful in drug and/or substrate tissue distribution assays. Tritiated, i.e. 3H, and carbon-14, i.e. 14 C, isotopes can be particularly beneficial for their ease of preparation and detectability. 11 C and 18 F isotopes are particularly useful in PET (positron emission tomography).

In the context of the present disclosure, the terms ‘individual’, ‘subject’, or ‘patient’ are used interchangeably to indicate an animal that may be suffering from a medical (pathological) condition and may be responsive to a compound/molecule, pharmaceutical drug, medical treatment or therapeutic treatment regimen of the disclosure. The animal is suitably a mammal, such as a human, cow, sheep, pig, dog, cat, bat, mouse or rat. In particular, the subject may be a human.

The term ‘alkyl’ refers to a monovalent, optionally substituted, saturated aliphatic hydrocarbon radical. Any number of carbon atoms may be present, but typically the number of carbon atoms in the alkyl group may be from 1 to about 20, from 1 to about 12, from 1 to about 6 or from 1 to about 4. Usefully, the number of carbon atoms is indicated, for example, a C1-C12 alkyl (or C1-C12 alkyl) refers to any alkyl group containing 1 to 12 carbon atoms in the chain. An alkyl group may be a straight chain (i.e. linear), branched chain, or cyclic. ‘Lower alkyl’ refers to an alkyl of 1 to 6 carbon atoms in the chain, and may have from 1 to 4 carbon atoms, or 1 to 2 carbon atoms. Thus, representative examples of lower alkyl radicals include methyl, ethyl, n-propyl, n-butyl, n-pentyl, n-hexyl, isopropyl, isobutyl, isopentyl, amyl (C₅H₁₁), sec-butyl, tert-butyl, sec-amyl, tert-pentyl, 2-ethylbutyl, 2,3-dimethylbutyl, and the like. ‘Higher alkyl’ refers to alkyls of 7 carbons and above, including n-heptyl, n-octyl, n-nonyl, n-decyl, n-dodecyl, n-tetradecyl, n-hexadecyl, n-octadecyl, n-eicosyl, and the like, along with branched variations thereof. A linear carbon chain of say 4 to 6 carbons would refer to the chain length not including any carbons residing on a branch, whereas in a branched chain it would refer to the total number. Optional substituents for alkyl and other groups are described herein.

The term ‘alkoxy’ or ‘alkoxyl’ as used herein refers to a monovalent radical of the formula RO—, where R is any alkyl, alkenyl or alkynyl as defined herein. Alkoxyl groups may be optionally substituted by any of the optional substituents described herein. ‘Lower alkoxyl’ has the formula RO—, where the R group is a lower alkyl, alkenyl or alkynyl. Representative alkoxy radicals include methoxy, ethoxy, n-propoxy, n-butoxy, n-pentyloxy, n-hexyloxy, isopropoxy, isobutoxy, isopentyloxy, amyloxy, sec-butoxy, tert-butoxy, tert-pentyloxy, and the like. Particularly exemplary alkoxyl groups are methoxyl and ethoxyl.

The term ‘cycloalkyl’ as used herein refers to a cyclized alkyl ring having the indicated number of carbon atoms in a specified range. Thus, for example, ‘C₃-C₆ cycloalkyl’ encompasses each of cyclopropyl, cyclobutyl, cyclopentyl, and cyclohexyl.

The term ‘aryl’ as used herein refers to a substituted or unsubstituted aromatic carbocyclic radical containing from 5 to about 15 carbon atoms (‘C6-C15 aryl’); and preferably 6 to 12 carbon atoms (‘C6-C12 aryl’). An aryl group may have only one individual carbon ring, or may comprise one or more fused rings in which at least one ring is aromatic in nature. A ‘phenyl’ is a radical formed by removal of a hydrogen atom from a benzene ring, and may be substituted or unsubstituted. A ‘phenoxyl’ group, therefore, is a radical of the formula RO—, wherein R is a phenyl radical. ‘Benzyl’ is a radical of the formula R—CH2-, wherein R is phenyl, and ‘benzyloxy’ is a radical of the formula RO—, wherein R is benzyl. The point of attachment to the base molecule on such fused aryl ring systems may be a C atom of the aromatic portion or a C or a N atom of the non-aromatic portion of the ring system. Non-limiting examples of aryl radicals include, phenyl, naphthyl, anthracenyl, benzyl, biphenyl, furanyl, pyridinyl, indanyl, anthraquinolyl, tetrahydronaphthyl, a benzoic acid radical, a furan-2-carboxylic acid radical, and the like.

The term ‘cycloaryl’ herein refers to a polycyclic group wherein an aryl group is fused to a 5- or 6-membered aliphatic ring. For example, C₆-C₁₂ cycloaryl means a C₆-C₁₂ aryl fused to a 5- or 6-membered aliphatic ring.

The term ‘heteroaryl’ as used herein refers to (i) a 5- or 6-membered ring having the characteristics of aromaticity containing at least one heteroatom selected from N, O and S, wherein each N is optionally in the form of an oxide, and (ii) a 9- or 10-membered bicyclic fused ring system, wherein the fused ring system of (ii) contains at least one heteroatom independently selected from N, O and S, wherein each ring in the fused ring system contains zero, one or more than one heteroatom, at least one ring is aromatic, each N is optionally in the form of an oxide, and each S in a ring which is not aromatic is optionally S(O) or S(O)₂. Typically, heteroaryl groups contain 5 to 14 ring atoms (‘5-14 membered heteroaryl’), and preferably 5 to 12 ring atoms (‘5-12 membered heteroaryl’). Heteroaryl rings are attached to the base molecule via a ring atom of the heteroaromatic ring, such that aromaticity is maintained. Suitable 5- and 6-membered heteroaromatic rings include, for example, pyridyl, 3-fluroropyridyl, 4-fluoropyridyl, 3-methoxypyridyl, 4-methoxypyridyl, pyrrolyl, pyrazinyl, pyrimidinyl, pyridazinyl, triazinyl, thienyl, furanyl, imidazolyl, pyrazolyl, triazolyl (i.e., 1,2,3-triazolyl or 1,2,4-triazolyl), tetrazolyl, oxazolyl, isooxazolyl, oxadiazolyl (i.e., the 1,2,3-, 1,2,4-, 1,2,5-(furazanyl), or 1,3,4-isomer), oxatriazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzisoxazolyl, benzoxazolyl, benzothiazolyl, chromenyl, quinolinyl, isoquinolinyl, benzopiperidinyl, benzofuranyl, imidazo[1,2-a]pyridinyl, benzotriazolyl, indazolyl, indolinyl, and isoindolinyl.

The term ‘heteroaryloxy’ or ‘heteroaryloxyl’ as used herein refers to an —O— heteroaryl group.

The terms ‘heterocycle’ or ‘heterocyclic’ group or ‘heterocyclyl’ as used herein refer to a monovalent radical of from about 4- to about 15-ring atoms, and preferably 3-, 4-, 5-, 6-, 7-, 8-, 9- or 10-ring members. Generally, the heterocyclic group contains one, two or three heteroatoms, selected independently from N, O and S. A preferred heteroatom is N. A heterocyclic group may have only one individual ring or may comprise more than one fused rings in which at least one ring contains a heteroatom. It may be fully saturated or partially saturated and may be substituted or unsubstituted as in the case or aryl and heteroaryl groups. In some embodiments the heterocyclic ring is a monocyclic ring having 5- or 6-members and at least one heteroatom selected from N, O and S, wherein each N is optionally in the form of an oxide. In some embodiments, the heterocyclic ring is a bicyclic ring having 9- or 10-members, wherein the fused ring system contains at least one heteroatom independently selected from N, O and S. In some embodiments, the heterocyclic ring is a tricyclic ring having 12- or 14-members, wherein the fused ring system contains at least one heteroatom independently selected from N, O and S. It should be appreciated that in bi and tricyclic ring systems each ring in the fused ring system may contain zero, one or more than one heteroatom, provided at least one ring contains a heteroatom, and wherein each N is optionally in the form of an oxide, and each S in a ring is optionally S(O) or S(O)₂.

Representative examples of unsaturated 5-membered heterocycles with only one heteroatom include 2- or 3-pyrrolyl, 2- or 3-furanyl, and 2- or 3-thiophenyl. Corresponding partially saturated or fully saturated radicals include 3-pyrrolin-2-yl, 2- or 3-pyrrolindinyl, 2- or 3-tetrahydrofuranyl, and 2- or 3-tetrahydrothiophenyl. Representative unsaturated 5-membered heterocyclic radicals having two heteroatoms include imidazolyl, oxazolyl, thiazolyl, pyrazolyl, and the like. The corresponding fully saturated and partially saturated radicals are also included. Representative examples of unsaturated 6-membered heterocyces with only one heteroatom include 2-, 3-, or 4-pyridinyl, 2H-pyranyl, and 4H-pryanyl. Corresponding partially saturated or fully saturated radicals include 2-, 3-, or 4-piperidinyl, 2-, 3-, or 4-tetrahydropyranyl and the like. Representative unsaturated 6-membered heterocyclic radicals having two heteroatoms include 3- or 4-pyridazinyl, 2-, 4-, or 5-pyrimidinyl, 2-pyrazinyl, morpholino, and the like. The corresponding fully saturated and partially saturated radicals are also included, e.g. 2-piperazine. The heterocyclic radical is bonded through an available carbon atom or heteroatom in the heterocyclic ring directly to the entity. Thus, a heterocyclic ring is attached to the base molecule via a ring atom of a saturated or unsaturated ring that contains a heteroatom. In some embodiments, where indicated, the heterocyclic group may be bonded to the entity through a linker such as an alkylene such as methylene or ethylene.

The disclosure encompasses fused ring systems, for example, ‘bicyclic’ or ‘tricyclic’ ring systems. In the context of the present disclosure, it is specifically intended that a fused ring system may include more than one fused aromatic ring, more than one fused non-aromatic/aliphatic ring, or one or more aromatic ring fused to one or more non-aromatic/aliphatic ring, such as a fusion of an aryl group with a cycloalkyl (or cycloalkenyl) group. Furthermore, it is intended that a fused ring system termed a bicyclic (or tricyclic) aryl is attached to the associated molecule via an aryl group, whereas a bicyclic (or tricyclic) cycloalkyl/cycloalkenyl is attached to the associated molecule via the cycloalkyl/cycloalkenyl group.

Similarly, in the context of fused ring systems, it is specifically intended that a bicyclic (or tricyclic) heteroaryl or heterocycloalkyl/heterocycloalkenyl need not contain heteroatoms in each of the fused ring systems. Rather, a bicyclic of tricyclic heteroaryl group may have one or more heteroatoms in any ring of the fused ring system, and not necessarily in the aryl ring that is the point of attachment to the associated molecule. Likewise, a bicyclic of tricyclic heterocycloalkyl or heterocycloalkenyl group may have one or more heteroatoms in any ring of the fused ring system, and not necessarily in the heterocycloalkyl or heterocycloalkenyl ring that is the point of attachment to the associated molecule.

The term ‘substituted’ means that one or more hydrogen atoms (attached to a carbon or heteroatom) is replaced with a selection from the indicated group of substituents, provided that the designated atom's normal valency under the existing circumstances is not exceeded. The group may be optionally substituted with particular substituents at positions that do not significantly interfere with the preparation of compounds falling within the scope of this invention and on the understanding that the substitution(s) does not significantly adversely affect the biological activity or structural stability of the compound. Combinations of substituents are permissible only if such combinations result in stable compounds. By ‘stable compound’ or ‘stable structure’, it is meant a compound that is sufficiently robust to survive isolation to a useful degree of purity from a reaction mixture and/or formulation into an efficacious therapeutic agent. The term ‘optionally substituted’ or ‘optional substituents’ as used herein means that the groups in question are either unsubstituted or substituted with one or more of the substituents specified. When the groups in question are substituted with more than one substituent, the substituents may be the same or different. Furthermore, the terms ‘independently’, ‘independently are’, and ‘independently selected from’ mean that the substituents in question may be the same or different.

The term ‘deuterium’ as used herein refers to an isotope of hydrogen that has one proton and one neutron in its nucleus and that has twice the mass of ordinary hydrogen. Deuterium herein is represented by the symbol ‘D’. The term ‘deuterated’ by itself or used to modify a compound or group as used herein refers to the presence of at least one deuterium atom attached to carbon. For example, the term ‘deuterated compound’ refers to a compound which contains one or more carbon-bound deuterium(s). In a deuterated compound of the present disclosure, when a particular position is designated as having deuterium, it is understood that the abundance of deuterium at that position is substantially greater than the natural abundance of deuterium, which is about 0.015%. The term ‘undeuterated’ or ‘non-deuterated’ as used herein refers to the ratio of deuterium atoms of which is not more than the natural isotopic deuterium content, which is about 0.015%; in other words, all hydrogen are present at their natural isotopic percentages. Unless otherwise stated, when a position is designated specifically as ‘H’ or ‘hydrogen’, the position is understood to have hydrogen at its natural abundance isotopic composition.

The term ‘isotopic enrichment factor’ as used herein refers to the ratio between the isotope abundance and the natural abundance of a specified isotope.

The term ‘isotopologue’ as used herein refers to a species in which the chemical structure differs from a specific compound of the invention only in the isotopic composition thereof.

The term ‘substantially free of other stereoisomers’ as used herein means less than 10% of other stereoisomers, preferably less than 5% of other stereoisomers, more preferably less than 2% of other stereoisomers and most preferably less than 1% of other stereoisomers are present.

The term ‘pharmaceutically acceptable salt’ as used herein refers to a salt that is not biologically or otherwise undesirable (e.g., not toxic or otherwise harmful). A salt of a compound of the invention is formed between an acid and a basic group of the compound, or a base and an acidic group of the compound. For example, when the compounds of the invention contain at least one basic group (i.e., groups that can be protonated), the invention includes the compounds in the form of their acid addition salts with organic or inorganic acids such as, for example, but not limited to salts with hydrogen chloride, hydrogen bromide, phosphoric acid, sulfuric acid, nitric acid, benzenesulfonic acid, acetic acid, citric acid, glutamic acid, lactic acid, and methanesulfonic acid. When compounds of the invention contain one or more acidic groups (e.g., a carboxylic acid), the invention includes the pharmaceutically acceptable salts of the compounds formed with but not limited to alkali metal salts, alkaline earth metal salts or ammonium salts. Examples of such salts include, but are not limited to, sodium salts, potassium salts, calcium salts, magnesium salts or salts with ammonia or organic amines such as, for example, ethylamine, ethanolamine, triethanolamine or amino acids. Additional examples of such salts can be found in Stahl, P. H. et al. Pharmaceutical Salts: Properties, Selection, and Use, 2nd Revised Edition, Wiley, 2011.

The terms ‘treatment’, ‘treating’ and ‘treat’ as used herein, include their generally accepted meanings, i.e., the management and care of a patient for the purpose of preventing, reducing the risk in incurring or developing a given condition or disease, prohibiting, restraining, alleviating, ameliorating, slowing, stopping, delaying, or reversing the progression or severity, and holding in check existing characteristics of a disease, disorder, or pathological condition, including the alleviation or relief of symptoms or complications, or the cure or elimination of the disease, disorder, or condition.

The term ‘therapeutically effective amount’ as used herein refers to that amount of compound of the invention that will elicit the biological or medical response of a tissue, system, animal, or human that is being sought by a researcher, veterinarian, medical doctor or other. As will be recognized by a person of ordinary skill in the art, a therapeutically effective amount of the compounds of the invention will vary and will depend on the disease treated, the severity of the disease, the route of administration, and the gender, age, and general health condition of the subject to whom the compound is being administered. The therapeutically effective amount may be administered as a single dose once a day, or as split doses administered multiple (e.g., two, three or four) times a day. The therapeutically effective amount may also be administered through continuous dosing, such as through infusion or with an implant.

Unless defined otherwise, ‘room temperature’ is intended to mean a temperature of from about 18 to 28° C., typically between about 18 and 25° C., and more typically between about 18 and 22° C. As used herein, the phrase ‘room temperature’ may be shortened to ‘rt’ or ‘RT’.

### Compounds:

Disclosed herein is a compound having the structural Formula 1:

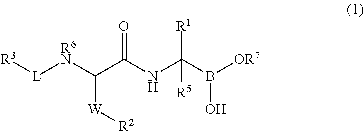

or a pharmaceutically acceptable salt, solvate, stereoisomer or mixture of stereoisomers, tautomer, isotopic form, pharmaceutically active metabolite thereof, or combinations thereof, wherein:

R¹ is selected from the group consisting of: deuterium, C1-C4 alkyl, C1-C4 alkoxyl, C1-C4 oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R⁸, CONR⁸R⁹, NR⁸R⁹, SR⁸, SO2NR⁸R⁹, C1-C4 alkyl, C1-C4 haloalkyl, C1-C4 alkoxy, phenyl, or a 5- or 6-membered heteroaryl;
W is C1-C4 alkyl, optionally substituted with one or more of deuterium, halogen, hydroxyl, CN, methyl or ethyl;
R² is a 5 to 14 membered heterocyclic mono-, bi- or tricyclic ring optionally having one or more heteroatoms selected from N, O and S, wherein the heterocyclic ring is optionally substituted with one or more substituent selected from deuterium, halogen, hydroxyl, CN, C1-C4 alkyl, C1-C4 haloalkyl or C1-C4 alkoxy;
L is C(O), C(O)O, C(O)NR⁴, S(O)₂, or a bond;
R³ is C₁-C₄ alkyl optionally substituted with one or more substituents each independently selected from the group consisting of deuterium, halogen, cyano, hydroxyl, C₁-C₄ alkoxyl, 5 or 6 membered aryl (e.g. phenyl) or 5 or 6 membered heteroaryl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C₁-C₄ alkoxyl, or C₁-C₄ alkyl that is optionally substituted with one to three substituents selected from deuterium, halogen, cyano, hydroxyl, or C₁-C₄ alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R⁸, CONR⁸R⁹, NR⁸R⁹, SR⁸, SO2NR⁸R⁹, C₁-C₄ alkoxyl, or C₁-C₄ alkyl that is optionally substituted with one to three substituents selected from deuterium, halogen, cyano, hydroxyl, or C₁-C₄ alkoxyl;
R⁴ is hydrogen, deuterium, or C1-C4 alkyl optionally substituted with one or more of halogen, hydroxyl and phenyl, wherein phenyl is optionally substituted with one or more substituent selected from halogen, hydroxyl and C1-C2 alkyl;
R⁵ is selected from hydrogen, deuterium or C1-C2 alkyl;
R⁶ is selected from hydrogen, deuterium or C1-C2 alkyl optionally substituted with one or more substituents each independently selected from the group consisting of halogen, hydroxyl, cyano, methoxyl and phenyl;
R⁷ is hydrogen, or R⁷ and R¹, together with the boron atom to which —OR⁷ is attached form a 5-membered heteroalkyl ring; and
R⁸ and R⁹ are each independently selected from hydrogen, deuterium, C1-C4 alkyl; C1-C4 haloalkyl, C1-C5 alkyl-alkoxyl, C3-C7 cycloalkyl, or R⁸ and R⁹ together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

In embodiments, R¹ is methyl, ethyl, n-propyl, i-propyl, n-butyl or tert-butyl, each optionally substituted with a phenyl ring. In some embodiments R¹ is suitably selected from methyl, n-propyl, n-butyl or tert-butyl. In various embodiments R¹ is selected from phenyl-(CH₂)₂— or phenyl-(CH₂)₃—. In certain embodiments, R¹ is methyl. In certain embodiments, R¹ is methyl substituted with a phenyl ring. In certain embodiments, R¹ is ethyl. In certain embodiments, R¹ is ethyl substituted with a phenyl ring. In certain embodiments, R¹ is n-propyl. In certain embodiments, R¹ is n-propyl substituted with a phenyl ring. In certain embodiments, R¹ is tert-butyl. In certain embodiments, R¹ is n-butyl. In certain embodiments, R¹ is methoxymethyl optionally substituted with a phenyl ring. In embodiments, R¹ is CO2H. In embodiments, R¹ is CO2R¹⁰. In embodiments, R¹ is CONR¹⁰R¹¹. In embodiments, R¹ is NR¹⁰R¹¹. In embodiments, R¹ is SR¹⁰. In embodiments, R¹ is SO2NR¹⁰R¹¹.

In embodiments, W is methyl, ethyl, n-propyl, n-butyl, each optionally substituted with one or more of deuterium, halogen, hydroxyl, CN, methyl or ethyl. In certain embodiments, W is C1-C2 alkyl, optionally substituted with one or more of deuterium, halogen, hydroxyl, CN, methyl or ethyl. In some embodiments, W is suitably selected from methyl or ethyl, each optionally substituted with one to three substituents selected from deuterium, F, Cl, hydroxyl or methyl deuterium, F, Cl, or hydroxyl. In certain embodiments, W is methyl. In certain embodiments, W is methyl substituted with a methyl group. In certain embodiments, W is ethyl. In certain embodiments, W is ethyl substituted with a methyl group.

In embodiments, R² is a 5 or 6 membered heterocyclic ring having one or more heteroatoms selected from N, O and S, wherein the heterocyclic ring is optionally substituted with one or more substituent selected from deuterium, halogen, hydroxyl, CN, C1-C4 alkyl, C1-C4 haloalkyl or C1-C4 alkoxy. In certain embodiments, R² is a 5 or 6 membered heterocyclic ring having one or two heteroatoms selected from N, O and S, wherein the heterocyclic ring is optionally substituted with one or more substituent selected from deuterium, halogen, hydroxyl, CN, methyl, ethyl, C1-C2 haloalkyl or C1-C2 alkoxy. In certain embodiments, R² is a 5 or 6 membered heterocyclic ring having one heteroatom selected from N, O and S, wherein the heterocyclic ring is optionally substituted with one or more substituent selected from F, Cl, hydroxyl, methyl, ethyl, wherein the methyl and ethyl are optionally substituted with one or more halogen or deuterium. In certain embodiments, R² is a 5 or 6 membered heterocyclic ring having two heteroatoms selected from N, O and S, wherein the heterocyclic ring is optionally substituted with one or more substituent selected from F, Cl, hydroxyl, methyl, ethyl, wherein the methyl and ethyl are optionally substituted with one or more halogen or deuterium.

In embodiments, R² is a 9 or 10 membered bicyclic heterocyclic ring having one or more heteroatoms selected from N, O and S, wherein the heterocyclic ring is optionally substituted with one or more substituent selected from deuterium, halogen, hydroxyl, CN, C1-C4 alkyl, C1-C4 haloalkyl or C1-C4 alkoxy. In certain embodiments, R² is a 9 or 10 membered heterocyclic ring having one or two heteroatoms selected from N, O and S, wherein the heterocyclic ring is optionally substituted with one or more substituent selected from deuterium, halogen, hydroxyl, CN, methyl, ethyl, C1-C2 haloalkyl or C1-C2 alkoxy. In certain embodiments, R² is a 9 or 10 membered heterocyclic ring having one heteroatom selected from N, O and S, wherein the heterocyclic ring is optionally substituted with one or more substituent selected from F, Cl, hydroxyl, methyl, ethyl, wherein the methyl and ethyl are optionally substituted with one or more halogen or deuterium. In certain embodiments, R² is a 9 or 10 membered heterocyclic ring having two heteroatoms selected from N, O and S, wherein the heterocyclic ring is optionally substituted with one or more substituent selected from F, Cl, hydroxyl, methyl, ethyl, wherein the methyl and ethyl are optionally substituted with one or more halogen or deuterium.

In embodiments, R² may have one, two, three or more heteroatoms, wherein: (i) the heteroatom or heteroatoms is N; or (ii) the heteroatom is selected from one or more of the group consisting of N and O; or (iii) the heteroatom is selected from one or more of the group consisting of N and S; or (iv) the heteroatom is selected from one or more of the group consisting of O and S. In some embodiments, R² has one heteroatom. In some embodiments, R² has two heteroatoms. In some embodiments, R² has three heteroatoms.

In embodiments, R² may be a heterocyclic ring selected from the group of: tetrahydrofuranyl, furanyl, pyrrolidinyl, pyrrolyl, thiophenyl, imidazolyl, pyrazolyl, oxazolyl, isooxazolyl, thiazolyl, isothiazolyl, oxadiazolyl, pyridinyl, piperdinyl, pyridazinyl, piperazinyl, pyrimidinyl, pyrazinyl, tetrahydropyranyl, pyranyl, dioxanyl, morpholinyl, azepanyl, oxepanyl, oxazepanyl, pyrrolizidinyl, indolyl, isoindolyl, indolizinyl, benzimidazolyl, purinyl, quinolinyl, isoquinolinyl, quinazolinyl, or pteridinyl, wherein the heterocyclic ring may be optionally substituted. In embodiments, the heterocyclic ring is joined to W via a carbon atom or via a heteroatom. R² may be joined to W via any suitable ring atom.

In embodiments, the R² group is selected from: 2-, 3-, 4- or 5-tetrahydrofuranyl, 2-, 3-, 4- or 5-furanyl, 1-, 2-, 3-, 4- or 5-pyrrolidinyl, 1-, 2-, 3-, 4- or 5-pyrrolyl, 2-, 3-, 4- or 5-thiophenyl, 1-, 2, 3-, 4- or 5-imidazolyl, 1-, 2-, 3-, 4- or 5-pyrazolyl, 2-, 3-, 4- or 5-oxazolyl, 2-, 3-, 4- or 5-isooxazolyl, 2-, 3-, 4- or 5-thiazolyl, 2-, 3-, 4- or 5-isothiazolyl, 2-, 3-, 4- or 5-oxadiazolyl, 1-, 2-, 3-, 4-, 5- or 6-pyridinyl, 1-, 2-, 3-, 4-, 5- or 6-piperidinyl, 1-, 2-, 3-, 4-, 5- or 6-pyridazinyl, 1-, 2-, 3-, 4-, 5- or 6-piperazinyl, 1-, 2-, 3-, 4-, 5- or 6-pyrimidinyl, 1-, 2-, 3-, 4-, 5- or 6-pyrazinyl, 1-, 2-, 3-, 4-, 5- or 6-tetrahydropyranyl, 2-, 3-, 4-, 5- or 6-pyranyl, 2-, 3-, 5- or 6-dioxanyl, 2-, 3-, 4-, 5- or 6-morpholinyl, 1-, 2-, 3-, 4-, 5-, 6- or 7-azepanyl, 2-, 3-, 4-, 5-, 6- or 7-oxepanyl, 2-, 3-, 4-, 5-, 6- or 7-oxazepanyl, 1-, 2-, 3-, 5-, 6-, 7- or 8-pyrrolizidinyl, 1-, 2-, 3-, 4-, 5-, 6- or 7-indolyl, 1-, 2-, 3-, 4-, 5-, 6-, 7-isoindolyl, 1-, 2-, 3-, 5-, 6-, 7- or 8-indolizinyl, 1-, 2-, 3-, 4-, 5-, 6- or 7-benzimidazolyl, 1-, 2-, 3-, 6-, 7-, 8- or 9-purinyl, 1-, 2-, 3-, 4-, 5-, 6-, 7- or 8-quinolinyl, 1-, 2-, 3-, 4-, 5-, 6-, 7- or 8-isoquinolinyl, 1-, 2-, 3-, 4-, 5-, 6-, 7- or 8-quinazolinyl, or 1-, 2-, 3-, 4-, 5-, 6-, 7- or 8-pteridinyl. In certain embodiments, the R² group is selected from: 2, 3- or 5-tetrahydrofuranyl, 2, 3- or 5-furanyl, 2—, 3- or 5-pyrrolidinyl, 2, 3- or 5-pyrrolyl, 2, 3- or 5-thiophenyl, 2, 3- or 5-imidazolyl, 2, 3- or 5-pyrazolyl, 2, 3- or 5-oxazolyl, 2, 3- or 5-isooxazolyl, 2, 3- or 5-thiazolyl, 2, 3- or 5-isothiazolyl, 2, 3- or 5-oxadiazolyl, 2, 3- or 4-pyridinyl, 2, 3- or 4-piperidinyl, 2, 3- or 4-pyridazinyl, 2, 3- or 4-piperazinyl, 2, 3- or 4-pyrimidinyl, 2, 3- or 4-pyrazinyl, 2, 3- or 4-tetrahydropyranyl, 2, 3- or 4-pyranyl, 2- or 3-dioxanyl, 2, 3- or 4-morpholinyl, 2, 3- or 4-azepanyl, 2, 3- or 4-oxepanyl, 2-, 3- or 4-oxazepanyl, 2, 3- or 5-pyrrolizidinyl, 2, 3- or 4-indolyl, 2, 3- or 4-isoindolyl, 2, 3- or 5-indolizinyl, 2, 3- or 4-benzimidazolyl, 2, 3- or 6-purinyl, 2, 3- or 4-quinolinyl, 2, 3- or 4-isoquinolinyl, 2, 3- or 4-quinazolinyl, or 2, 3- or 4-pteridinyl.

In certain suitable embodiments, R² is a heterocyclic ring selected from: imidazolyl, pyrazolyl, oxazolyl, thiazolyl or indolyl. In embodiments, R² is selected from: 2-imidazolyl, 3-pyrazolyl, 2-oxazolyl, 5-oxazolyl, 2-thiazolyl or 3-indolyl.

In embodiments, R² is substituted with methyl. In certain embodiments, the methyl substituent is attached to a carbon atom of the heterocyclic ring. In certain embodiments, the methyl substituent is attached to a heteroatom, particularly N, of the heterocyclic ring.

In particular embodiments, R² is a heterocyclic ring selected from: 1-methyl-2-imidazolyl, 1-methyl-3-pyrazolyl, 2-oxazolyl, 5-oxazolyl, 2-thiazolyl or 3-indolyl. In certain embodiments, R² is 1-methyl-2-imidazolyl. In certain embodiments, R² is 1-methyl-3-pyrazolyl. In certain embodiments, R² is 2-oxazolyl. In certain embodiments, R² is 5-oxazolyl. In certain embodiments, R² is 2-thiazolyl. In certain embodiments, R² is 3-indolyl.

In embodiments, L is selected from C(O), C(O)O, C(O)NH, C(O)N(CH₃) or SO₂. Suitably, L may be selected from C(O), C(O)O and C(O)NH. In certain embodiments, L is C(O). In certain embodiments, L is a bond. In certain embodiments, L is C(O)O. In certain embodiments, L is C(O)NR⁴. In certain embodiments, L is SO₂.

In embodiments, R³ is selected from C1-C4 alkyl, a 5- or 6-membered heteroaryl, C6 aryl, a 5- or 6-membered heterocycloalkyl and C6 cycloalkyl. R³ is optionally substituted. In some embodiments, R³ is selected from methyl, ethyl, n-propyl, i-propyl, n-butyl or tert-butyl, each optionally substituted with a phenyl ring. In various embodiments, R³ is selected from methyl, i-propyl and tert-butyl. In some suitable embodiments, R³ is selected from phenyl, phenyl-(CH₂)— and phenyl-(CH₂)₂—, wherein the phenyl group is optionally substituted. In particular embodiments, R³ may be selected from an aryl, heteroaryl, cycloalkyl or heterocycloalkyl selected from tetrahydropyranyl, pyrazinyl, tetrahydropyrrolyl, tetrahydrofuranyl, tetrahydropyranyl, cyclohexanyl, oxazolyl and morpholinyl, wherein said aryl, heteroaryl, cycloalkyl or heterocycloalkyl is optionally substituted. In such embodiments, R³ may be selected from n-tetrahydropyrrolyl, n-morpholinyl and pyrazinyl. In particular embodiments, R³ is pyrazinyl. In any such embodiments, the substituent on said R³ group may be selected from one to three of halogen, hydroxyl and C1-C2 alkyl. In particular, the substituent may be selected from one or two of Cl, hydroxyl and methyl. In some embodiments, R³ is selected from 2-chlorophenyl, 3-chlorophenyl, 2,5-dichlorophenyl, 2,4-dimethyloxazolyl, and 3-hydroxy n-tetrahydropyrrolyl.

In particular embodiments, R³ is pyrazinyl.

In certain embodiments, R³ may be C1-C4 alkyl optionally substituted with one or more substituents independently selected from the groups consisting of fluoro, chloro, cyano, or methoxyl; or R³ may be cycloalkyl, heterocyclyl, aryl, cycloaryl, or heteroaryl, any of which is optionally substituted with one or more fluoro, chloro, cyano, methoxyl, or C₁-C₄ alkyl that is optionally substituted with one to three fluoro, chloro, cyano, or methoxyl. In certain embodiments, R³ is C₁-C₄ alkyl optionally substituted with one or more substituents each independently selected from the groups consisting of fluoro, chloro, cyano, or methoxyl. In certain embodiments, R³ is cycloalkyl which is optionally substituted with one or more fluoro, chloro, cyano, methoxyl, or C₁-C₄ alkyl that is optionally substituted with one to three fluoro, chloro, cyano, or methoxyl. In certain embodiments, R³ is heterocyclyl which is optionally substituted with one or more fluoro, chloro, cyano, methoxyl, or C₁-C₄ alkyl that is optionally substituted with one to three fluoro, chloro, cyano, or methoxyl. In certain embodiments, R³ is aryl which is optionally substituted with one or more fluoro, chloro, cyano, methoxyl, or C₁-C₄ alkyl that is optionally substituted with one to three fluoro, chloro, cyano, or methoxyl. In certain embodiments, R³ is cycloaryl which is optionally substituted with one or more fluoro, chloro, cyano, methoxyl, or C₁-C₄ alkyl that is optionally substituted with one to three fluoro, chloro, cyano, or methoxylln certain embodiments, R³ is heteroaryl which is optionally substituted with one or more fluoro, chloro, cyano, methoxyl, or C₁-C₄ alkyl that is optionally substituted with one to three fluoro, chloro, cyano, or methoxyl. In certain embodiments, R³ is methyl, tert-butyl, trifluoromethyl, phenyl optionally substituted with one or more fluoro, chloro, cyano, methoxyl, or C₁-C₄ alkyl that is optionally substituted with one to three fluoro, chloro, cyano, or methoxyl; pyrdinyl optionally substituted with one or more fluoro, chloro, cyano, methoxyl, or C₁-C₄ alkyl that is optionally substituted with one to three fluoro, chloro, cyano, or methoxyl; piperidinyl optionally substituted with one or more fluoro, chloro, cyano, methoxyl, or C₁-C₄ alkyl that is optionally substituted with one to three fluoro, chloro, cyano, or methoxyl; pyrrolidinyl optionally substituted with one or more fluoro, chloro, cyano, methoxyl, or C₁-C₄ alkyl that is optionally substituted with one to three fluoro, chloro, cyano, or methoxyl; imidazolyl optionally substituted with one or more fluoro, chloro, cyano, methoxyl, or C₁-C₄ alkyl that is optionally substituted with one to three fluoro, chloro, cyano, or methoxyl; pyrazolyl optionally substituted with one or more fluoro, chloro, cyano, methoxyl, or C₁-C₄ alkyl that is optionally substituted with one to three fluoro, chloro, cyano, or methoxyl; thiazolyl optionally substituted with one or more fluoro, chloro, cyano, methoxyl, or C₁-C₄ alkyl that is optionally substituted with one to three fluoro, chloro, cyano, or methoxyl; pyrazinyl optionally substituted with one or more fluoro, chloro, cyano, methoxyl, or C₁-C₄ alkyl that is optionally substituted with one to three fluoro, chloro, cyano, or methoxyl; oxazolyl optionally substituted with one or more fluoro, chloro, cyano, methoxyl, or C₁-C₄ alkyl that is optionally substituted with one to three fluoro, chloro, cyano, or methoxyl; or morpholinyl optionally substituted with one or more fluoro, chloro, cyano, methoxyl, or C₁-C₄ alkyl that is optionally substituted with one to three fluoro, chloro, cyano, or methoxyl.

In certain embodiments, R³ is methyl. In certain embodiments, R³ is tert-butyl. In certain embodiments, R³ is trifluoromethyl. In certain embodiments, R³ is phenyl optionally substituted with one or more fluoro, chloro, cyano, methoxyl, or C₁-C₄ alkyl that is optionally substituted with one to three fluoro, chloro, cyano, or methoxyl. In certain embodiments, R³ is pyridinyl optionally substituted with one or more fluoro, chloro, cyano, methoxyl, or C₁-C₄ alkyl that is optionally substituted with one to three fluoro, chloro, cyano, or methoxyl. In certain embodiments, R³ is piperidinyl optionally substituted with one or more fluoro, chloro, cyano, methoxyl, or C₁-C₄ alkyl that is optionally substituted with one to three fluoro, chloro, cyano, or methoxyl. In certain embodiments, R³ is pyrrolidinyl optionally substituted with one or more fluoro, chloro, cyano, methoxyl, or C₁-C₄ alkyl that is optionally substituted with one to three fluoro, chloro, cyano, or methoxyl. In certain embodiments, R³ is imidazolyl optionally substituted with one or more fluoro, chloro, cyano, methoxyl, or C₁-C₄ alkyl that is optionally substituted with one to three fluoro, chloro, cyano, or methoxyl. In certain embodiments, R³ is pyrazolyl optionally substituted with one or more fluoro, chloro, cyano, methoxyl, or C₁-C₄ alkyl that is optionally substituted with one to three fluoro, chloro, cyano, or methoxyl. In certain embodiments, R³ is thiazolyl optionally substituted with one or more fluoro, chloro, cyano, methoxyl, or C₁-C₄ alkyl that is optionally substituted with one to three fluoro, chloro, cyano, or methoxyl. In certain embodiments, R³ is pyrazinyl optionally substituted with one or more fluoro, chloro, cyano, methoxyl, or C₁-C₄ alkyl that is optionally substituted with one to three fluoro, chloro, cyano, or methoxyl. In certain embodiments, R³ is oxazolyl optionally substituted with one or more fluoro, chloro, cyano, methoxyl, or C₁-C₄ alkyl that is optionally substituted with one to three fluoro, chloro, cyano, or methoxyl. In certain embodiments, R³ is morpholinyl optionally substituted with one or more fluoro, chloro, cyano, methoxyl, or C₁-C₄ alkyl that is optionally substituted with one to three fluoro, chloro, cyano, or methoxyl.

In embodiments, R³ is CO2H. In embodiments, R³ is CO2R⁸. In embodiments, R³ is CONR⁸R⁹. In embodiments, R³ is NR⁸R⁹. In embodiments, R³ is SR⁸. In embodiments, R³ is SO2NR⁸R⁹.

In embodiments, R⁸ and R⁹ are each independently selected from hydrogen, deuterium, C1-C4 alkyl; C1-C4 haloalkyl or C1-C5 alkyl-alkoxyl. In embodiments, R⁸ and R⁹ are each independently selected from hydrogen, deuterium, C1-C4 alkyl; C1-C4 haloalkyl, C1-C5 alkyl-alkoxyl or C3-C7 cycloalkyl. In embodiments, R⁸ and R⁹ are each independently selected from hydrogen, deuterium, C1-C2 alkyl; C1-C2 haloalkyl, C1-C2 alkyl-alkoxyl or C3-C5 cycloalkyl. In embodiments, R⁸ and R⁹ together with the N to which they are attached form 3 to 7 membered heterocyclic ring optionally having one or more additional heteroatoms selected from N, O and S. In any such embodiments, the C3-C7 cycloalkyl, C3-C5 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl. In embodiments, the substituents may be selected from one, two or three of deuterium, F, Cl, hydroxyl, oxo, CN, C1-C2 alkyl, C1-C2 haloalkyl or C1-C2 alkoxyl.

In embodiments, R⁴ is selected from hydrogen and methyl.

In embodiments, R⁵ is selected from hydrogen or C1-C2 alkyl. In embodiments, R⁵ is hydrogen. In embodiments, R⁵ is deuterium. In embodiments, R⁵ is C1-C2 alkyl. In other embodiments, In embodiments, R⁵ is methyl. In other embodiments, R⁵ is ethyl.

In embodiments, R⁶ is selected from hydrogen, phenyl-(CH₂)— and phenyl-(CH₂)₂—. In particular embodiments, R⁶ is hydrogen.

In particular embodiments, R⁷ is hydrogen.

In aspects and embodiments, the compound of the disclosure may have the structural formula 2:

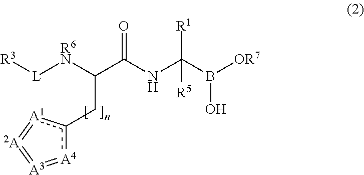

wherein:
n is 1, 2 or 3;
each of A¹ to A⁴ are independently selected from C(R⁸), N(R⁹), N, O or S;
R⁸ is selected from hydrogen, deuterium, halogen, hydroxyl, CN, C1-C4 alkyl, C1-C4 haloalkyl or C1-C4 alkoxy;
R⁹ is selected from hydrogen, deuterium, C1-C4 alkyl or C1-C4 haloalkyl; and R¹ and R³ to R⁷, and L are as defined elsewhere herein.

In embodiments of the compound of formula 2, A¹ is selected from N(R⁹), N, O or S. In embodiments of the compound of formula 2, A² is selected from N(R⁹), N, O or S. In embodiments of the compound of formula 2, A³ is selected from N(R⁹), N, O or S. In embodiments of the compound of formula 2, A⁴ is selected from N(R⁹), N, O or S.

In embodiments of the compound of formula 2, one, two or three of A¹ to A⁴ are C(R⁸). Typically two or three of A¹ to A⁴ are C(R⁸). In some embodiments, two of A¹ to A⁴ are C(R⁸). In some embodiments, three of A¹ to A⁴ are C(R⁸).

In embodiments of the compound of formula 2, A¹ is selected from N(R⁸) or N; and A² is selected from N(R⁹) or N. In embodiments of the compound of formula 2, A¹ is selected from N(R⁹) or N; and A⁴ is selected from N(R⁹) or N. In embodiments of the compound of formula 2, A¹ is selected from N(R⁹) or N; and A⁴ is selected from O or S. In embodiments of the compound of formula 2, A² is selected from N(R⁹) or N; and A⁴ is selected from O or S. In embodiments of the compound of formula 2, A² is C(R⁸); and A³ is C(R⁸).

In embodiments of the compound of formula 2, R⁸ is selected from hydrogen, F, Cl, hydroxyl, methyl, ethyl, CF₃, or OMe. In embodiments of the compound of formula 2, R⁸ is selected from hydrogen or methyl.

In embodiments of the compound of formula 2, R⁹ is selected from hydrogen, methyl, ethyl or OMe. In embodiments of the compound of formula 2, R⁹ is selected from hydrogen or methyl.

In embodiments of the compound of formula 2, n is suitably 1 or 2. In particular embodiments of the compound of formula 2, n is 1.

In any of the aspects and embodiments disclosed herein, halogen may suitably be selected from fluoro or chloro. In particular, in any such embodiments, halogen may be chloro.

In another embodiment, the present invention is directed to a compound, or a pharmaceutically acceptable salt thereof, represented by any one of the following structures:

TABLE 1
Chemical structures of compounds according to the disclosure.
Any of these compounds may also exist in the isomeric forms,
particularly the oxaborolane isomer derivative, and such
isomers are explicitly intended to be encompassed within the
scope of this disclosure. While some stereochemistry has been
depicted, in some cases where stereoisomers exist,
stereochemistry may be arbitrarily assigned.
Structure 1:

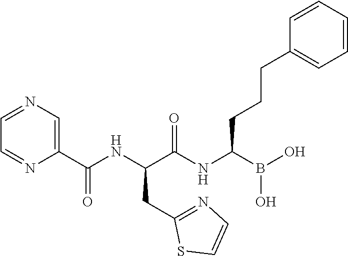 [SMILES: O=C(N[C@H](Cc1nccs1)C(=O)N[C@@H](CCCc1ccccc1)B(O)O)c1cnccn1]

Structure 2:

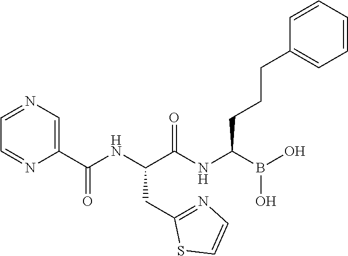 [SMILES: O=C(N[C@@H](Cc1nccs1)C(=O)N[C@@H](CCCc1ccccc1)B(O)O)c1cnccn1]

Structure 3:

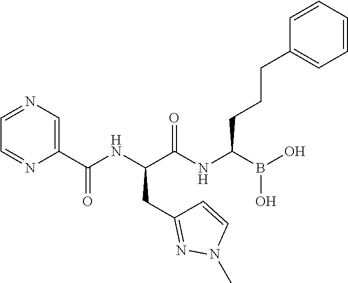 [SMILES: Cn1ccc(C[C@@H](NC(=O)c2cnccn2)C(=O)N[C@@H](CCCc2ccccc2)B(O)O)n1]

Structure 4:

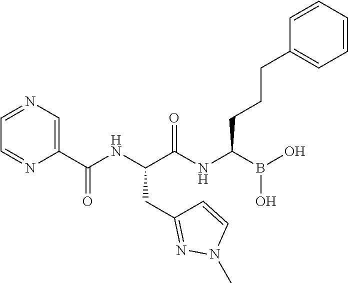 [SMILES: Cn1ccc(C[C@H](NC(=O)c2cnccn2)C(=O)N[C@@H](CCCc2ccccc2)B(O)O)n1]

Structure 5:

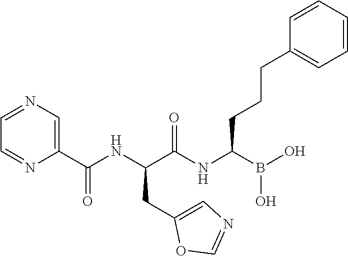 [SMILES: O=C(N[C@H](Cc1cnco1)C(=O)N[C@@H](CCCc1ccccc1)B(O)O)c1cnccn1]

Structure 6:

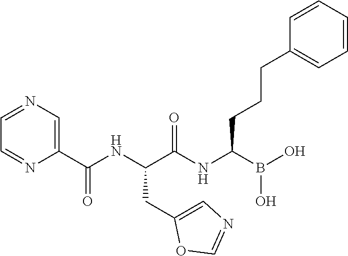 [SMILES: O=C(N[C@@H](Cc1cnco1)C(=O)N[C@@H](CCCc1ccccc1)B(O)O)c1cnccn1]

Structure 7:

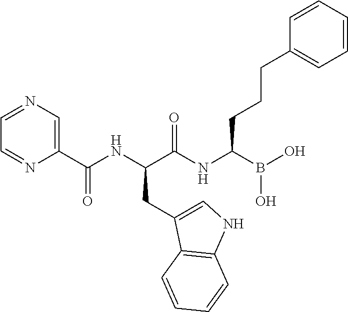 [SMILES: O=C(N[C@H](Cc1c[nH]c2ccccc12)C(=O)N[C@@H](CCCc1ccccc1)B(O)O)c1cnccn1]

Structure 8:

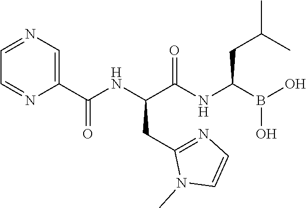 [SMILES: CC(C)C[C@H](NC(=O)[C@@H](Cc1nccn1C)NC(=O)c1cnccn1)B(O)O]

Structure 9:

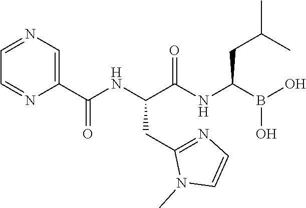 [SMILES: CC(C)C[C@H](NC(=O)[C@H](Cc1nccn1C)NC(=O)c1cnccn1)B(O)O]

Structure 10:

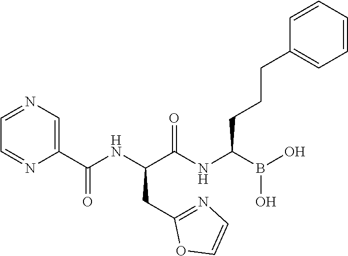 [SMILES: O=C(N[C@H](Cc1ncco1)C(=O)N[C@@H](CCCc1ccccc1)B(O)O)c1cnccn1]

Structure 11:

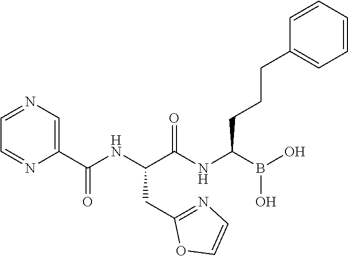 [SMILES: O=C(N[C@@H](Cc1ncco1)C(=O)N[C@@H](CCCc1ccccc1)B(O)O)c1cnccn1]

Structure 12:

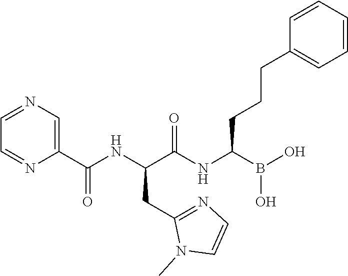 [SMILES: Cn1ccnc1C[C@@H](NC(=O)c1cnccn1)C(=O)N[C@@H](CCCc1ccccc1)B(O)O]

Structure 13:

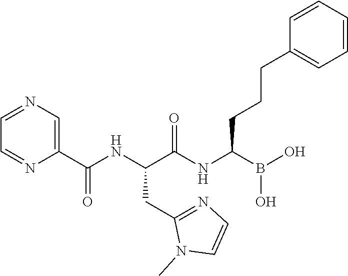 [SMILES: Cn1ccnc1C[C@H](NC(=O)c1cnccn1)C(=O)N[C@@H](CCCc1ccccc1)B(O)O]

The compounds of the present invention may contain asymmetric carbon atoms (sometimes as the result of a deuterium atom) and thereby can exist as either individual stereoisomers or mixtures of enantiomers or mixtures of diastereomers. Accordingly, a compound of the present invention may exist as either a racemic mixture, a mixture of diastereomers, or as individual stereoisomers that are substantially free of other stereoisomers. Synthetic, separation, or purification methods to be used to obtain an enantiomer of a given compound are known in the art and are applicable for obtaining the compounds identified herein.

Unless otherwise indicated, when a disclosed compound is named or depicted by a structure without specifying the stereochemistry and has one or more chiral centers, it is understood to represent all possible stereoisomers of the compound. In other words, chiral centers that lack solid wedged or hashed wedged bonds indicate a mixture of stereoisomers.

Certain compounds of the present invention may be able to exist as tautomers. All tautomeric forms of these compounds, whether isolated individually or in mixtures, are within the scope of the present invention. For example, in instances where an —OH substituent is permitted on a heteroaromatic ring and keto-enol tautomerism is possible, it is understood that the substituent might in fact be present, in whole or in part, in the oxo (═O) form.

Compounds of the present invention may exist in amorphous form and/or one or more crystalline forms. As such, all amorphous and crystalline forms and mixtures thereof of the compounds of the invention are intended to be included within the scope of the present invention. In addition, some of the compounds of the present invention may form solvates with water (i.e., a hydrate) or common organic solvents. Such solvates and hydrates, particularly the pharmaceutically acceptable solvates and hydrates, of the compounds of this invention are likewise encompassed within the scope of the compounds of the invention and the pharmaceutically acceptable salts thereof, along with un-solvated and anhydrous forms of such compounds.

In one embodiment, deuterium isotope content at the deuterium substituted position is greater than the natural isotopic deuterium content (0.015%), more preferably greater than 50%, more preferably greater than 60%, more preferably greater than 75%, more preferably greater than 90%, more preferably greater than 95%, more preferably greater than 97%, more preferably greater than 99%. It will be understood that some variation of natural isotopic abundance may occur in any compound depending upon the source of the reagents used in the synthesis. Thus, a preparation of undeuterated compounds may inherently contain small amounts of deuterated isotopologues, such amounts being insignificant as compared to the degree of stable isotopic substitution of the deuterated compounds of the invention (see, e.g., Gannes, L. Z. et al., Comp. Biochem. Physiol. Mol. Integr. Physiol, 119, 725 (1998)). Replacement of hydrogen with deuterium may affect the activity, toxicity, and pharmacokinetics (e.g., absorption, distribution, metabolism, and excretion (“ADME”)) of some drugs. For instance, such replacement may alter the chemical stability and biochemical reactivity of a compound through kinetic isotope effects. Because of the increased mass of deuterium relative to hydrogen, epimerization at stereogenic carbons may be slowed down when hydrogen is replaced with deuterium (see Pirali, T. et al, J. Med. Chem. 62, 5276-97 (2019)). Additionally, the presence of deuterium may affect how a molecule interacts with enzymes, thereby impacting enzyme kinetics. While in certain cases the increased mass of deuterium as compared to hydrogen can stabilize a compound and thereby improve activity, toxicity, or half-life, such impact is not predictable. In other instances deuteration may have little to no impact on these properties, or may affect them in an undesirable manner. Whether and/or how such replacement will impact drug properties can only be determined if the drug is synthesized, evaluated, and compared to its non-deuterated counterpart (see Fukuto, J. M., et al., J. Med. Chem. 34, 2871-76 (1991)). Because some drugs have multiple sites of metabolism or more than one active sites for binding to a target, it is unpredictable as to which sites may benefit by deuterium replacement or to what extent isotope enrichment is necessary to produce a beneficial effect.

A further embodiment of the present invention are compounds of the invention (that is, compounds of Compounds 1, 2, 3, 4, 5, 6, 7, 8, 9, 10, 11, 12 and 13) or their pharmaceutically acceptable salts wherein one or more hydrogen is substituted with a deuterium atom.

Additional embodiments of the invention are pharmaceutical compositions comprising a compound of the disclosure (that is, compounds of Compounds 1, 2, 3, 4, 5, 6, 7, 8, 9, 10, 11, 12 and 13) or a pharmaceutically acceptable salt thereof and a pharmaceutically acceptable excipient.

Further embodiments of the invention are methods of treating a disease characterized by mitochondrial dysfunction, such methods comprising administering to a subject in need thereof a therapeutically effective amount of a compound of the invention, or a pharmaceutically acceptable salt thereof. In some embodiments, the disease is selected from the group consisting of Alper's syndrome (Alpers-Huttenlocher syndrome), ataxia neuropathy syndrome (ANS), Mitochondrial DNA Depletion Syndrome (MDDS), Leigh Syndrome (Leigh Disease), Leber's Hereditary Optic Neuropathy (LHON), chronic progressive external ophthalmoplegia (CPEO), myoclonic epilepsy myopathy sensory ataxia (MEMSA), MELAS (Mitochondrial Encephalopathy, Lactic Acidosis, and Stroke-like episodes) syndrome, MERRF (myoclonus epilepsy with ragged-red fibers) syndrome, mitochondrial neurogastrointestinal encephalomyopathy (MNGIE), neuropathy, ataxia, and retinitis pigmentosa (NARP), Kearn's-Sayre Syndrome (KSS), and Pearson's Syndrome.

In some embodiments, the disease to be treated with a compounds of the invention or pharmaceutically acceptable salts thereof is associated with mtDNA mutations or deletions, for example, m.3243A>G, m.11778G>A, m.14484T>C, m.3460G>A, m.8344A>G, m.3271T>C, m.3251A>G, m.8356T>C, m.4274T>C, m.14709T>C, m.12320A>G, m.4269A>G, m.12258C>A, m.1606G>A, m.10010T>C, m.7445A>G, and m.1555A>G (see https://mitomap.org/MITOMAP).

Additional embodiments of the invention are methods of treating cancers, such as those identified in Wong, K. S. et al. “Recent Advances in Targeting Human Mitochondrial AAA+ Proteases to Develop Novel Cancer Therapeutics,” Advances in Experimental Medicine and Biology, 1158,119-142 (2019), using a compound of the invention or its pharmaceutically acceptable salt.

For the treatment of cancer, the compounds described herein may be administered in combination with a chemotherapeutic agent. Therapeutically effective amounts of the additional chemotherapeutic agent(s) are well known to those skilled in the art. However, it is well within the attending physician to determine the amount of other chemotherapeutic agent(s) to be delivered.

Examples of these chemotherapeutic agents include, but are not limited to, Abitrexate (Methotrexate Injection), Abraxane (Paclitaxel Injection), Actemra (Tocilizumab), Adcetris (Brentuximab Vedotin Injection), Adriamycin (Doxorubicin), Adrucil Injection (5-FU (fluorouracil)), Afinitor (Everolimus), Afinitor Disperz (Everolimus), Aldara (Imiquimod), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Avelumab, Bexxar (Tositumomab), BiCNU (Carmustine), Blenoxane (Bleomycin), Blincyto (Blinatumomab), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Cyramza (Ramucirumab), Dacogen (Decitabine), Darzalex (Daratumumab), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Durvalumab, Eligard (Leuprolide), Ellence (Ellence (epirubicin)), Eloxatin (Eloxatin (oxaliplatin)), Elspar (Asparaginase), Emcyt (Estramustine), Empliciti (Elotuzumab), Enhertu (fam-trastuzumab deruxtecan-nxki), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gazyva (Obinutuzumab), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leflunomide (SU101), Lartruvo (Olaratumab), Leukeran (Chlorambucil), Leukine (Sargramostim), Leustatin (Cladribine), Libtayo (Cemiplimab), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Ninlaro (Ixazomib), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Portrazza (Necitumumab), Prednisone Intensol (Prednisone), Proleukin (Aldesleukin), Purinethol (Mercaptopurine), Reclast (Zoledronic acid), Revlimid (Lenalidomide), Removab (Catumaxomab), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Sarclisa (Isatuximab-irfc), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Tecentriq (Atezolizumab), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Tremelimumab, Trelstar (Triptorelin Injection), Trexall (Methotrexate), Trisenox (Arsenic trioxide), Tykerb (lapatinib), Unituxin (Dinutuximab), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (lbritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MED14736, MPDL3280A and MSB0010718C targeting.

Further embodiments of the invention are methods of treating neurodegenerative disorders, metabolic disorders, and diseases associated with the aging process.

### Dosage Forms, Medicaments and Pharmaceuticals:

The compounds, molecules or agents of the disclosure may be used to treat (e.g. cure, alleviate or prevent) one or more diseases, infections or disorders. Thus, in accordance with the disclosure, the compounds and molecules may be manufactured into medicaments or may be incorporated or formulated into pharmaceutical compositions.

The molecules, compounds and compositions of the disclosure may be administered by any convenient route, for example, methods of administration include intradermal, intramuscular, intraperitoneal, intravenous, subcutaneous, intranasal, epidural, oral, sublingual, intranasal, intravaginal, transdermal, rectally, by inhalation, or topically to the skin. Delivery systems are also known to include, for example, encapsulation in liposomes, microgels, microparticles, microcapsules, capsules, etc. Any other suitable delivery system known in the art is also envisioned in use. Administration can be systemic or local. The mode of administration may be left to the discretion of the practitioner.

The dosage administered will, of course, vary depending upon known factors, such as the pharmacodynamic properties of the particular active agent; the chosen mode and route of administration; the age, health and weight of the recipient; the nature of the disease or disorder to be treated; the extent of the symptoms; any simultaneous or concurrent treatments; the frequency of treatment; and the effect desired.

Depending on known factors, such as those noted above, the required dosage of the active agent may be administered in a single daily dose, or the total daily dosage may be administered in divided doses of e.g. two, three, or four times daily. Suitably, the therapeutic treatment regime according to the disclosure is devised for a single daily dose or for a divided daily dose of two doses.

The ‘effective amount’ or ‘therapeutically effective amount’ is meant to describe an amount of compound or a composition of the disclosure that is effective in curing, inhibiting, alleviating, reducing or preventing the adverse effects of the diseases or disorders to be treated, or the amount necessary to achieve a physiological or biochemically-detectable effect. Thus, at the effective amount, the compound or agent is able to produce the desired therapeutic, ameliorative, inhibitory or preventative effect in relation to disease or disorder. Beneficially, an effective amount of the compound or composition of the disclosure may have the effect of inhibiting LONP1. Diseases or disorders which may benefit from LONP1 inhibition include, for example, proliferative diseases or disorders and cancer.

When administered to a subject, a compound of the disclosure is suitably administered as a component of a composition that comprises a pharmaceutically acceptable carrier or vehicle. One or more additional pharmaceutical acceptable carrier (such as diluents, adjuvants, excipients or vehicles) may be combined with the compound of the disclosure in a pharmaceutical composition. Suitable pharmaceutical carriers are described in “Remington's Pharmaceutical Sciences” by E. W. Martin. Pharmaceutical formulations and compositions of the disclosure are formulated to conform to regulatory standards and according to the chosen route of administration.

Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are generally sterile. Water is a suitable vehicle when the compound is to be administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents.

The medicaments and pharmaceutical compositions of the disclosure can take the form of solutions, suspensions, emulsion, tablets, pills, pellets, powders, gels, capsules (for example, capsules containing liquids or powders), modified-release formulations (such as slow or sustained-release formulations), suppositories, emulsions, aerosols, sprays, suspensions, or any other form suitable for use. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

Suitably, the therapeutic compositions or medicaments of the disclosure are formulated in accordance with routine procedures as a pharmaceutical composition adapted for oral administration (more suitably for humans). Compositions for oral delivery may be in the form of tablets, lozenges, aqueous or oily suspensions, granules, powders, emulsions, capsules, syrups, or elixirs, for example. Thus, in one embodiment, the pharmaceutically acceptable vehicle is a capsule, tablet or pill.

Orally administered compositions may contain one or more agents, for example, sweetening agents such as fructose, aspartame or saccharin; flavouring agents such as peppermint, oil of wintergreen, or cherry; colouring agents; and preserving agents, to provide a pharmaceutically palatable preparation. When the composition is in the form of a tablet or pill, the compositions may be coated to delay disintegration and absorption in the gastrointestinal tract, so as to provide a sustained release of active agent over an extended period of time. Selectively permeable membranes surrounding an osmotically active driving compound are also suitable for orally administered compositions. In these dosage forms, fluid from the environment surrounding the capsule is imbibed by the driving compound, which swells to displace the agent or agent composition through an aperture. These dosage forms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. For oral formulations, the location of release may be the stomach, the small intestine (the duodenum, the jejunum, or the ileum), or the large intestine. One skilled in the art is able to prepare formulations that will not dissolve in the stomach yet will release the material in the duodenum or elsewhere in the intestine. Suitably, the release will avoid the deleterious effects of the stomach environment, either by protection of the compound (or composition) or by release of the compound (or composition) beyond the stomach environment, such as in the intestine. To ensure full gastric resistance a coating impermeable to at least pH 5.0 would be essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac, which may be used as mixed films.

While it can be beneficial to provide therapeutic compositions and/or compounds of the disclosure in a form suitable for oral administration, for example, to improve patient compliance and for ease of administration, in some embodiments, compounds or compositions of the disclosure may cause undesirable side-effects, such as intestinal inflammation which may lead to premature termination of a therapeutic treatment regime. Thus, in some embodiments, the therapeutic treatment regime is adapted to accommodate ‘treatment holidays’, e.g. one or more days of non-administration. For example, treatment regimens and therapeutic methods of the disclosure may comprise a repetitive process comprising administration of the therapeutic composition or compound for a number of consecutive days, followed by a treatment holiday of one or more consecutive days. For example, a treatment regime of the disclosure may comprise a repetitive cycle of administration of the therapeutic composition or compound for between 1 and 49 consecutive days, between 2 and 42 days, between 3 and 35 days, between 4 and 28 days, between 5 and 21 days, between 6 and 14 days, or between 7 and 10 days; followed by a treatment holiday of between 1 and 14 consecutive days, between 1 and 12 days, between 1 and 10 days, or between 1 and 7 days (e.g. 1, 2, 3, 4, 5, 6 or 7 days).

To aid dissolution of the therapeutic agent into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. Potential nonionic detergents that could be included in the formulation as surfactants include: lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants, when used, could be present in the formulation of the compound or derivative either alone or as a mixture in different ratios.

Typically, compositions for intravenous administration comprise sterile isotonic aqueous buffer. Where necessary, the compositions may also include a solubilising agent.

Another suitable route of administration for the therapeutic compositions of the disclosure is via pulmonary or nasal delivery.

Additives may be included to enhance cellular uptake of the therapeutic agent of the disclosure, such as the fatty acids oleic acid, linoleic acid and linolenic acid.

The therapeutic agents of the disclosure may also be formulated into compositions for topical application to the skin of a subject.

Where the invention provides more than one active compound/agent for use in combination, generally, the agents may be formulated separately or in a single dosage form, depending on the prescribed most suitable administration regime for each of the agents concerned. When the therapeutic agents are formulated separately, the pharmaceutical compositions of the invention may be used in a treatment regime involving simultaneous, separate or sequential administration with the other one or more therapeutic agent. The other therapeutic agent(s) may comprise a compound of the disclosure or a therapeutic agent known in the art.

Specific and general embodiments of the disclosure will now be described by way of the following non-limiting examples.

### EXAMPLES

The examples and preparations provided below further illustrate and exemplify the compounds of the present invention and methods of preparing such compounds. It is to be understood that the scope of the present invention is not limited in any way by the scope of the following examples and preparations.

The structures of the compounds are confirmed by MS, elemental analysis and/or NMR, where peaks assigned to the characteristic protons in the title compound are presented where appropriate. 1H NMR shift (6) are given in parts per million (ppm) down field from an internal reference standard.

Preparative SFC method: Separation was performed on PIC-SOLUTION-175 instrument by using Reflect (R,R) WHELK-01 column (21.1 mm×250 mm), 5μ operating at 35° C., maintaining flow rate of 60 ml/min, using 65% C02 in super critical state and 35% methanol as mobile phase, run for 12 minutes at 100 bar (detection at 230 nm).

The following abbreviations are used in the synthetic schemes above and the examples below. If an abbreviation used herein is not defined, it has its generally accepted meaning:
Abu 2-aminobutyric acid
ACN Acetonitrile
Bn Benzyl
BnBr Benzyl bromide
CuCl2 Copper chloride
D2O Deuterated water
DCM Dichloromethane
DIPEA Diisopropylethylamine
DMSO Dimethylsulfoxide
DMSO-d6 Deuterated dimethylsulfoxide
Et Ethyl
EtOAc Ethyl acetate
Et3N Triethylamine
EtOH Ethanol
Et2O Diethyl ether
h hour
H2O water
HBr Hydrobromic acid
HCl Hydrochloric acid
I2 Iodine
IBCF Isobutyl chloroformate
K2CO3 Potassium carbonate
KOAc Potassium acetate
KOtBu Potassium tert-butoxide
LAF Laminar Flow Box
LiOH Lithium hydroxide
min minutes
Me Methyl
MeCN Acetonitrile
MeI Methyl iodide
MeMgBr Methyl magnesium bromide
MeO or OMe Methoxy
MeOD Deuterated Methanol
MeOH Methanol
MgSO4 Magnesium sulfate
MS Mass spectrometry
N2 Nitrogen
NaH Sodium hydride
NMM N-Methylmorpholine
NaOAc Sodium acetate
NaOH Sodium hydroxide
Na2CO3 Sodium carbonate
Na2SO4 Sodium sulfate
NaH Sodium hydride
NaHCO₃Sodium bicarbonate
NH4Cl Ammonium chloride
Pd2(dba)3 Tris(dibenzylideneacetone)dipalladium(0)
POCl3 Phosphoryl chloride
Prep-HPLC Preparative high performance liquid chromatography
Prep-TLC Preparative thin layer chromatography
PTSA p-Toluenesulfonic acid
Ph Phenyl
rt Room temperature (typically 18 to 22° C.)
SiO2 Silica gel
t-BuOH Tert-butanol
THF Tetrahydrofuran

### Preparation of the Compounds:

The starting materials and reagents used in each step in the preparation are known and can be readily prepared or purchased from commercial sources.

The compound obtained in each step can also be used for the next reaction as a reaction mixture thereof, or after obtaining a crude product thereof. Alternatively, the compound obtained in each step can be isolated and/or purified from the reaction mixture by a separation means such as concentration, crystallization, recrystallization, distillation, solvent extraction, fractionation, chromatography and the like according to a conventional method.

In each reaction step, while the reaction time varies depending on the reagents and solvents to be used, unless otherwise specified, it is generally 1 min to 48 hr, preferably 10 min to 8 hr.

In the reaction of each step, while the reaction temperature varies depending on the reagents and solvents to be used, unless otherwise specified, it is generally −78° C. to 300° C., preferably −78° C. to 150° C.

In the reaction of each step, unless otherwise specified, a reagent is used in 0.5 equivalent to 20 equivalents, preferably 0.8 equivalent to 5 equivalents, relative to the substrate. When a reagent is used as a catalyst, the reagent is used in 0.001 equivalent to 1 equivalent, preferably 0.01 equivalent to 0.2 equivalent, relative to the substrate. When the reagent is also a reaction solvent, the reagent is used in a solvent amount.

In the reaction of each step, unless otherwise specified, it is performed without solvent or by dissolving or suspending in a suitable solvent. Specific examples of the solvent include the following. Alcohols: methanol, ethanol, tert-butyl alcohol, 2-methoxyethanol and the like; ethers: diethyl ether, diphenyl ether, tetrahydrofuran, 1,2-dimethoxyethane and the like; aromatic hydrocarbons: chlorobenzene, toluene, xylene and the like; saturated hydrocarbons: cyclohexane, hexane and the like; amides: N,N-dimethylformamide, N-methylpyrrolidone and the like; halogenated hydrocarbons: dichloromethane, carbon tetrachloride and the like; nitriles: acetonitrile and the like; sulfoxides: dimethyl sulfoxide and the like; aromatic organic bases: pyridine and the like; acid anhydrides: acetic anhydride and the like; organic acids: formic acid, acetic acid, trifluoroacetic acid and the like; inorganic acids: hydrochloric acid, sulfuric acid and the like; esters: ethyl acetate and the like; ketones: acetone, methyl ethyl ketone and the like; and water. Two or more kinds of the above-mentioned solvents may be used by mixing at an appropriate ratio.

Unless otherwise specified, the reaction of each step is performed according to a known method, for example, the methods described in “Reactions and Syntheses: In the Organic Chemistry Laboratory 2nd Edition” (Lutz F. Tietze, Theophil Eicher, Ulf Diederichsen, Andreas Speicher, Nina Schützenmeister) Wiley, 2015; “Organic Syntheses Collective Volumes 1-12” (John Wiley & Sons Inc); “Comprehensive Organic Transformations, Third Edition” (Richard C. Larock) Wiley, 2018 and the like.

In each step, protection or deprotection of a functional group is performed by a known method, for example, the methods described in “Protective Groups in Organic Synthesis, 4^{th} Ed.” (Theodora W. Greene, Peter G. M. Wuts) Wiley-Interscience, 2007; “Protecting Groups 3rd Ed.” (P. J. Kocienski) Thieme, 2004 and the like.

Deuterated LONP1 inhibitors of the present invention can be prepared using chemical reactions known to a person of ordinary skill in the art using deuterated starting materials or reagents.

Deuterium-containing reagents are well known in the art and can be prepared using known procedures or purchased from commercial sources. The deuterated compounds obtained can be characterized by analytical techniques known to persons of ordinary skill in the art. For example, nuclear magnetic resonance (‘NMR’) can be used to determine a compound's structure while mass spectroscopy (‘MS’) can be used to determine the amount of deuterium atom in the compound by comparison to its non-deuterated form.

The Examples and preparations provided below further illustrate and exemplify the compounds of the present invention and methods of preparing such compounds. It is to be understood that the scope of the present invention is not limited in any way by the scope of the following examples and preparations.

The structures of the compounds are confirmed by either elemental analysis or NMR, where peaks assigned to the characteristic protons in the title compound are presented where appropriate. ¹H NMR shift (δH) are given in parts per million (ppm) down field from an internal reference standard.

### Example 1 and Example 2

### Synthesis of (R)-4-phenyl-1-((R)-2-(pyrazine-2-carboxamido)-3-(thiazol-2-yl) propanamido) butyl)boronic acid (Compound 1) and ((R)-4-phenyl-1-((S)-2-(pyrazine-2-carboxamido)-3-(thiazol-2-yl)propanamido)butyl)boronic acid (Compound 2)

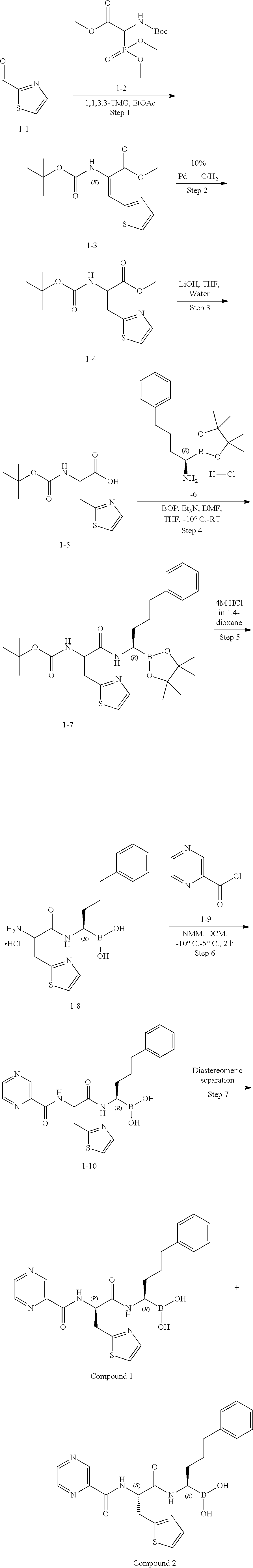

Synthesis of methyl (E)-2-((tert-butoxycarbonyl)amino)-3-(thiazol-2-yl)acrylate [Step 1]: To a stirred solution of methyl 2-(tert-butoxycarbonylamino)-2-dimethoxyphosphoryl-acetate (1-2, 5.05 g, 17.0 mmol) and thiazole-2-carbaldehyde (1-1, 2.00 g, 17.5 mmol) in THF (20 mL)) at ice-cold condition, 1, 1, 3, 3-tetramethylguanidine (2.4 mL, 19.3 mmol) was added dropwise over 5 mins and the mixture was stirred for 2 h at room temperature. The reaction mixture was concentrated under reduced pressure and the residue was partitioned between water and ethyl acetate. The organic layer was separated and washed with aqueous NH₄Cl solution (50 mL). Organic layer was dried over Na₂SO₄, filtered and concentrated under reduced pressure to afford product which was purified by combiflash column chromatography by (0-30% ethyl acetate/hexane) to afford methyl (E)-2-((tert-butoxycarbonyl)amino)-3-(thiazol-2-yl)acrylate (1-3, 2.00 g). [M+H]⁺=285.

Synthesis of methyl 2-((tert-butoxycarbonyl)amino)-3-(thiazol-2-yl)propanoate [Step 2]: To the stirred solution of methyl (Z)-2-(tert-butoxycarbonylamino)-3-thiazol-2-yl-prop-2-enoate (1-3, 2.00 g, 7.03 mmol) in methanol (20 mL) was added Pd—C (20% on activated carbon, 450 mg) and the reaction mixture was stirred at room temperature for 16 h under H₂ balloon pressure. The reaction mixture was filtered through celite bed using 50 ml DCM. Combined filtrate was concentrated under reduced pressure to afford methyl 2-(tert-butoxycarbonylamino)-3-(thiazol-2-yl)propanoate (1-4, 1.90 g). [M+H]⁺=287.

Synthesis of 2-((tert-butoxycarbonyl)amino)-3-(thiazol-2-yl)propanoic acid [Step 3]: To the stirred solution of methyl 2-(tert-butoxycarbonylamino)-3-thiazol-2-yl-propanoate (1-4, 1.90 g, 6.64 mmol) in THF (10 mL) and water (2 mL)) was added LiOH H₂O (139 mg, 5.82 mmol) at ice cold condition and stirred for 2 h at room temperature. The reaction mixture was concentrated under reduced and diluted with 10 ml water. The aqueous part was washed with EtOAc. The aqueous part was acidified with 1N aq. HCl (pH=2) and lyophilized to afford 2-(tert-butoxycarbonylamino)-3-thiazol-2-yl-propanoic acid (1-5, 1.4 g). [M+H]⁺=273; ¹H NMR (400 MHz, DMSO-_{d6}) δ: 12.83 (s, 1H), 7.71 (d, 1H), 7.59 (d, 1H), 7.22 (d, 1H), 4.31-4.30 (m, 1H), 3.43-3.39 (m, 1H), 3.38-3.25 (m, 1H), 1.35 (m, 10H).

Synthesis of tert-butyl (1-oxo-1-(((R)-4-phenyl-1-(4, 4, 5, 5-tetramethyl-1, 3, 2-dioxaborolan-2-yl)butyl)amino)-3-(thiazol-2-yl)propan-2-yl)carbamate [Step 4]: To the stirred solution of 2-(tert-butoxycarbonylamino)-3-thiazol-2-yl-propanoic acid (1-5, 400 mg, 1.47 mmol) in THF (5 mL), Et₃N (0.60 mL, 4.41 mmol) and (1R)-4-phenyl-1-(4, 4, 5, 5-tetramethyl-1, 3, 2-dioxaborolan-2-yl)butan-1-amine hydrochloride (1-6, 504 mg, 1.62 mmol) were added at −10° C., followed by the addition of BOP (974 mg, 2.20 mmol). The resulting reaction mixture was allowed to stir for 2 h at room temperature. The reaction mixture was diluted with water and extracted with EtOAc. Organic layer was washed with brine solution, dried over Na₂SO₄, filtered and evaporated under reduced pressure to get tert-butyl (1-oxo-1-(((R)-4-phenyl-1-(4, 4, 5, 5-tetramethyl-1, 3, 2-dioxaborolan-2-yl)butyl)amino)-3-(thiazol-2-yl)propan-2-yl)carbamate (1-7, 750 mg). [M−H]⁻=528.

Synthesis of 2-amino-N—((R)-4-phenyl-1-(4, 4, 5, 5-tetramethyl-1, 3, 2-dioxaborolan-2-yl)butyl)-3-(thiazol-2-yl)propanamide hydrochloride [Step 5]: To the stirred solution of fert-butyl N-[2-oxo-2-[[(1R)-4-phenyl-1-(4, 4, 5, 5-tetramethyl-1, 3, 2-dioxaborolan-2-yl)butyl]amino]-1-(thiazol-2-ylmethyl)ethyl]carbamate (1-7, 780 mg, 1.47 mmol) in 1, 4-dioxane (10 mL) was added HCl (4M in dioxane, 3.67 mL, 14.7 mmol) at ice cold condition stirred at room temperature for 2 h. Reaction mixture was evaporated under reduced pressure, washed with pentane and dried to get 2-amino-N-[(1R)-4-phenyl-1-(4, 4, 5, 5-tetramethyl-1, 3, 2-dioxaborolan-2-yl)butyl]-3-thiazol-2-yl-propanamide hydrochloride (1-8, 600 mg). [M−H]⁻=346.

Synthesis of (R)-4-phenyl-1-((R)-2-(pyrazine-2-carboxamido)-3-(thiazol-2-yl)propanamido) butyl)boronic acid (Compound 1), and ((R)-4-phenyl-1-((S)-2-(pyrazine-2-carboxamido)-3-(thiazol-2-yl)propanamido)butyl)boronic acid (Compound 2) [Step 6]: To the stirred solution of 2-amino-N-[(1R)-4-phenyl-1-(4, 4, 5, 5-tetramethyl-1, 3, 2-dioxaborolan-2-yl)butyl]-3-thiazol-2-yl-propanamide hydrochloride (1-8, 200 mg, 0.43 mmol) in DCM (10 mL), NMM (0.07 mL, 0.52 mmol) was added at 0° C. and the reaction mixture was stirred at this temperature for 5 min. Next pyrazine-2-carbonyl chloride (1-9, 73 mg, 0.52 mmol) was added and the reaction mixture was stirred at room temperature for 2 h. The reaction mixture was diluted with DCM and washed with water and brine. The organic part was dried over Na₂SO₄, filtered and evaporated under reduced pressure at 30° C. to obtain product (1-10), which was purified by RP prep HPLC purification. The pure fraction was lyophilized to afford [(1R)-4-phenyl-1-[(2R)-2-(pyrazine-2-carbonylamino)-3-thiazol-2-yl-propanoyl]amino]butyl]boronic acid as diastereomeric mixture (1-10, 150 mg). The mixture was further purified by normal phase preparative chiral HPLC to afford ((R)-4-phenyl-1-((R)-2-(pyrazine-2-carboxamido)-3-(thiazol-2-yl)propanamido)butyl)boronic acid, Peak 1 (Compound 1, 23 mg) and ((R)-4-phenyl-1-((S)-2-(pyrazine-2-carboxamido)-3-(thiazol-2-yl)propanamido) butyl)boronic acid, Peak 2 (Compound 2, 10 mg). Absolute stereochemistry of each isomer has been arbitrarily assigned.

Compound 1: [M−H]⁻=452; ¹H NMR (400 MHz, MeOD) δ: 9.19 (s, 1H), 8.79 (d, 1H), 8.70 (s, 1H), 7.70 (br s, 1H), 7.45 (br s, 1H), 7.20-7.18 (m, 2H), 7.14-7.10 (m, 3H), 5.30 (t, 1H), 3.71-3.69 (m, 2H), 2.62-2.55 (s, 3H), 1.6-1.27 (m, 4H).

Compound 2: [M−H]⁻=452; ¹H NMR (400 MHz, MeOD) δ: 9.20 (s, 1H), 8.79 (d, 1H), 8.69 (s, 1H), 7.70 (br s, 1H), 7.45 (br s, 1H), 7.22-7.18 (m, 2H), 7.14-7.08 (m, 3H), 5.29 (t, 1H), 3.75-3.63 (m, 2H), 2.65-2.62 (m, 1H), 2.59-2.54 (m, 2H), 1.59-1.40 (m, 4H).

Chiral-PREP Method: Chiral separation was performed on an Agilent 1200 series instrument. Column: CHIRALPAK IC (250×21 mm) 5μ, operating at ambient temperature and flow rate of 21.0 mL/min. The mobile phase was 0.1% TFA in the mixture of 80% hexane, 10% dichloromethane, and 10% isopropanol, and held isocratic for up to 30 min with detection at a wavelength of 268 nm.

### Example 3 and Example 4

### Synthesis of ((R)-1-((R)-3-(1-methyl-1H-pyrazol-3-yl)-2-(pyrazine-2-carboxamido) propanamido)-4-phenylbutyl)boronic acid (Compound 3), and ((S)-1-((R)-3-(1-methyl-1H-pyrazol-3-yl)-2-(pyrazine-2-carboxamido)propanamido)-4-phenylbutyl)boronic acid (Compound 4)

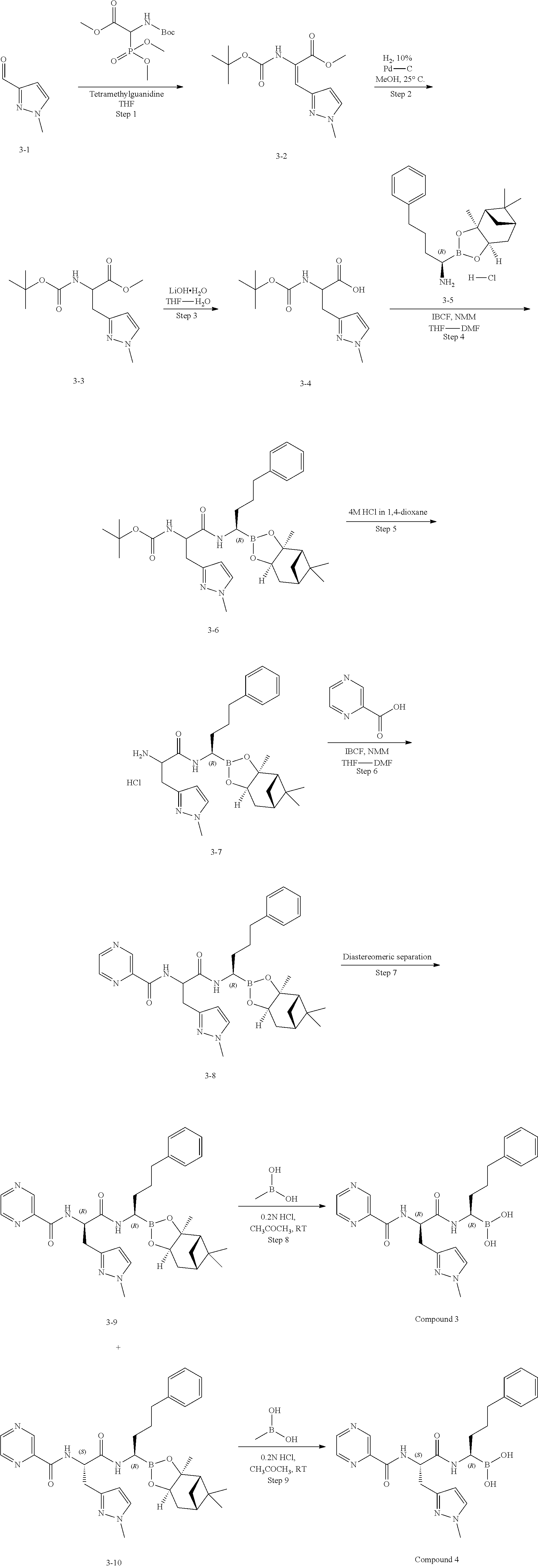

Synthesis of methyl (E)-2-((tert-butoxycarbonyl)amino)-3-(1-methyl-1H-pyrazol-3-yl) acrylate [Step 1]: To a stirred solution of 1-methylpyrazole-3-carbaldehyde (3-1, 500 mg, 4.5 mmol) and methyl 2-((tert-butoxycarbonyl)amino)-2-(dimethoxyphosphoryl)acetate (1.3 g, 4.4 mmol) in THF (5 mL) was added 1, 1, 3, 3-tetramethylguanidine (0.6 mL, 5.0 mmol) dropwise over one minute at 0° C. After stirring for 2 h at 25° C., the reaction mixture was concentrated under reduced pressure. The product was dissolved in ethyl acetate, washed with brine, dried over anhy. Na₂SO₄, filtered and concentrated under reduced pressure to get methyl (E)-2-((tert-butoxycarbonyl)amino)-3-(1-methyl-1H-pyrazol-3-yl)acrylate (3-2, 1.0 g). The product was used in the next step without further purification. [M+H]⁺=282.

Synthesis of methyl 2-((tert-butoxycarbonyl)amino)-3-(1-methyl-1H-pyrazol-3-yl) propanoate [Step 2]: To a solution of methyl (E)-2-((tert-butoxycarbonyl)amino)-3-(1-methyl-1H-pyrazol-3-yl)acrylate (3-2, 1.0 g, 3.5 mmol) in methanol (10 mL) was added Pd/C (250 mg, 25 wt %) under N₂. The reaction vessel was evacuated and backfilled with H₂ twice and finally kept under hydrogen atmosphere. After stirring for 16 h at 25° C., the reaction mixture was filtered through a pad of celite. The filtrate was concentrated to dryness to afford methyl 2-((tert-butoxycarbonyl)amino)-3-(1-methyl-1H-pyrazol-3-yl)propanoate (3-3, 800 mg), which was used in the next step without further purification. [M+H]⁺=284.

Synthesis of 2-((tert-butoxycarbonyl)amino)-3-(1-methyl-1H-pyrazol-3-yl)propanoic acid [Step 3]: To a stirred solution of methyl 2-((tert-butoxycarbonyl)amino)-3-(1-methyl-1H-pyrazol-3-yl)propanoate (3-3, 800 mg, 2.8 mmol) in THF (8 mL)-water (1 mL) was added LiOH·H₂O (101 mg, 4.2 mmol) and the mixture was stirred at 25° C. After 2 h, the reaction mixture was concentrated and diluted with 10 ml water. The aqueous phase was washed with EtOAc, acidified with 1N HCl (pH=2), and lyophilized to afford 2-((tert-butoxycarbonyl)amino)-3-(1-methyl-1H-pyrazol-3-yl)propanoic acid (3-4, 700 mg). The product was forwarded to the next step without further purification. [M+H]⁺=270.

Synthesis of tert-butyl (3-(1-methyl-1H-pyrazol-3-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl) amino)propan-2-yl)carbamate [Step 4]: To a solution of 2-((tert-butoxycarbonyl)amino)-3-(1-methyl-1H-pyrazol-3-yl)propanoic acid (3-4, 505 mg, 1.9 mmol) in THF (5 mL) was added IBCF (0.2 mL, 1.9 mmol) followed by NMM (0.2 mL, 1.9 mmol) at −15° C. After 45 min, a solution of (R)-4-phenyl-1-((3aS, 4S, 6S, 7aR)-3a, 5, 5-trimethylhexahydro-4, 6-methanobenzo[d][1, 3, 2]dioxaborol-2-yl)butan-1-amine hydrochloride (3-5,620 mg, 1.7 mmol) in DMF (1 mL) was added dropwise followed by NMM (0.2 mL, 1.7 mmol). After stirring for 1 h at the same temperature, the reaction mixture was diluted with EtOAc and washed successively with 0.1 N aq. HCl (×2), 5% aq. K₂CO₃ (×2), water (×2) and brine (×2). The organic phase was dried over anhy. Na₂SO₄, filtered and concentrated under reduced pressure to get tert-butyl (3-(1-methyl-1H-pyrazol-3-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)carbamate (3-6, 700 mg), which was directly used for the next step without further purification. [M−H]⁻=577.

Synthesis of 2-amino-3-(1-methyl-1H-pyrazol-3-yl)-N—((R)-4-phenyl-1-((3aS, 4S, 6S, 7aR)-3a, 5, 5-trimethylhexahydro-4, 6-methanobenzo[d][1, 3, 2]dioxaborol-2-yl)butyl)propanamide hydrochloride [Step 5]: To a solution of tert-butyl (3-(1-methyl-1H-pyrazol-3-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)carbamate (3-6, 700 mg, 1.2 mmol) in 1, 4-dioxane (5 mL) was added HCl (3.0 mL, 4M in dioxane, 12.0 mmol) at 0° C., and the mixture was stirred at 25° C. for 16 h. The reaction mixture was concentrated under reduced pressure to afford 2-amino-3-(1-methyl-1H-pyrazol-3-yl)-N—((R)-4-phenyl-1-((3aS, 4S, 6S, 7aR)-3a, 5, 5-trimethylhexahydro-4, 6-methanobenzo[d][1, 3, 2]dioxaborol-2-yl)butyl)propanamide hydrochloride (3-7, 550 mg), which was used in the next step without further purification. [M−H]⁻=477.

Synthesis of N-(3-(1-methyl-1H-pyrazol-3-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl) amino)propan-2-yl)pyrazine-2-carboxamide [Step 6]: To a solution of pyrazine-2-carboxylic acid (146 mg, 1.2 mmol) in THF (5 mL) was added IBCF (0.2 mL, 1.2 mmol) followed by NMM (0.2 mL, 1.2 mmol) at −15° C. After 45 min, a solution of 2-amino-3-(1-methyl-1H-pyrazol-3-yl)-N—((R)-4-phenyl-1-((3aS, 4S, 6S, 7aR)-3a, 5, 5-trimethylhexahydro-4, 6-methanobenzo[d][1, 3, 2]dioxaborol-2-yl)butyl)propanamide hydrochloride (3-7, 550 mg, 1.1 mmol) in DMF (1 mL) was added dropwise followed by NMM (0.2 mL, 1.1 mmol). After stirring for 1 h at the same temperature, the reaction mixture was diluted with EtOAc, and washed successively with 0.1 N aq. HCl (×2), 5% aq. K₂CO₃, water and brine. The organic phase was dried over anhy. Na2SO4, filtered and concentrated under reduced pressure to afford N-(3-(1-methyl-1H-pyrazol-3-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide (3-8, 500 mg). [M+H]⁺=586.

Isolation of N—((R)-3-(1-methyl-1H-pyrazol-3-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino) propan-2-yl)pyrazine-2-carboxamide and N—((S)-3-(1-methyl-1H-pyrazol-3-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide [Step 7]: N-(3-(1-methyl-1H-pyrazol-3-yl)-1-oxo-1-(((R)-4-phenyl-1-((3aS, 4S, 6S, 7aR)-3a, 5, 5-trimethylhexa hydro-4, 6-methanobenzo[d][1, 3, 2]dioxaborol-2-yl)butyl)amino)propan-2-yl)pyrazine-2-carboxamide (3-8, 500 mg, 1.0 mmol) was separated by a chiral HPLC to afford N—((R)-3-(1-methyl-1H-pyrazol-3-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide, Peak 2 (3-9, 89 mg) and N—((S)-3-(1-methyl-1H-pyrazol-3-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide, Peak 1 (3-10, 100 mg).

N—((R)-3-(1-methyl-1H-pyrazol-3-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl) pyrazine-2-carboxamide, the (R,R)-diastereomer, was synthesized separately following the same reaction scheme but using asymmetric hydrogenation in Step 2. The analytical data for the (R,R)-diastereomer matched that of Peak 2 (3-9), and thus the corresponding (S,R)-diastereomer was assigned to Peak 1 (3-10).
3-9: [M−H]⁻=583.
3-10: [M−H]⁻=583.

Chiral SFC purification method: Chiral separation was performed on a Thar SFC-80 series instrument using a CHIRALPAK AS-H column (21 mm×250 mm), 5μ, operating at 35° C. temperature, maintaining a flow rate of 40 gm/min, using 85% CO₂ in super critical state and 15% of 0.3% isopropylamine in (ethanol:methanol 70:30) as mobile phase, isocratic up to 12 min and isobaric at 100 bar, with detection at a wavelength of 213 nm.

Synthesis of ((R)-1-((R)-3-(1-methyl-1H-pyrazol-3-yl)-2-(pyrazine-2-carboxamido) propanamido)-4-phenylbutyl)boronic acid (Compound 3) [Step 8]: To a solution of N—((R)-3-(1-methyl-1H-pyrazol-3-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide (3-9, 100 mg, 0.2 mmol) and methylboronic acid (154 mg, 2.6 mmol) in acetone (4 mL) was added 0.2N aq. HCl (4.0 mL, 0.2 mmol) at 25° C. After stirring for 16 h at the same temperature, the reaction mixture was concentrated under reduced pressure, and then lyophilized. The material was purified by prep HPLC (RP) and lyophilized to afford ((R)-1-((R)-3-(1-methyl-1H-pyrazol-3-yl)-2-(pyrazine-2-carboxamido)propanamido)-4-phenylbutyl) boronic acid (Compound 3, 40 mg). [M−H]⁻=449; ¹H NMR (400 MHz, MeOD): δ 9.19 (s, 1H), 8.79-8.78 (m, 1H), 8.69 (s, 1H), 7.40-7.41 (m, 1H), 7.23-7.10 (m, 5H), 6.15-6.14 (m, 1H), 5.05 (t, 1H), 3.79 (s, 3H), 3.30-3.24 (m, 2H), 2.61-2.55 (m, 3H), 1.59-1.54 (m, 4H).

Synthesis of ((S)-1-((R)-3-(1-methyl-1H-pyrazol-3-yl)-2-(pyrazine-2-carboxamido) propanamido)-4-phenylbutyl)boronic acid (Compound 4) [Step 9]: To a solution of N—((S)-3-(1-methyl-1H-pyrazol-3-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide (3-10, 89 mg, 0.2 mmol) and methylboronic acid (137 mg, 2.3 mmol) in acetone (4 mL) was added 0.2N aq. HCl (4.0 mL, 0.2 mmol) at 25° C. After stirring for 16 h at the same temperature, the reaction mixture was concentrated under reduced pressure, and then lyophilized. The material was purified by prep HPLC (RP) and lyophilized to afford ((S)-1-((R)-3-(1-methyl-1H-pyrazol-3-yl)-2-(pyrazine-2-carboxamido)propanamido)-4-phenylbutyl) boronic acid (Compound 4.15 mg). [M−H]⁻=449; ¹H NMR (400 MHz, MeOD): δ 9.19 (s, 1H), 8.79-8.78 (m, 1H), 8.69 (s, 1H), 7.42-7.41 (m, 1H), 7.23-7.10 (m, 5H), 6.15-6.14 (m, 1H), 5.05 (t, 1H), 3.79 (t, 3H), 3.24-3.23 (m, 2H), 2.61-2.55 (m, 3H), 1.62-1.39 (m, 4H).

### Example 5 and Example 6

### Synthesis of ((R)-1-((R)-3-(oxazol-5-yl)-2-(pyrazine-2-carboxamido)propanamido)-4-phenylbutyl)boronic acid (Compound 5), and (R)-1-((S)-3-(oxazol-5-yl)-2-(pyrazine-2-carboxamido)propanamido)-4-phenylbutyl)boronic acid (Compound 6)

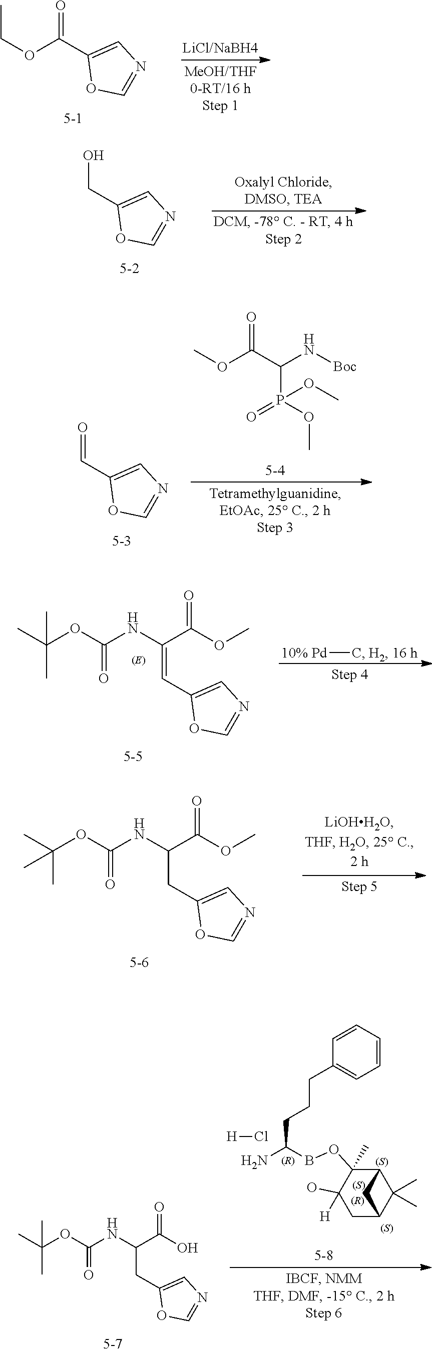

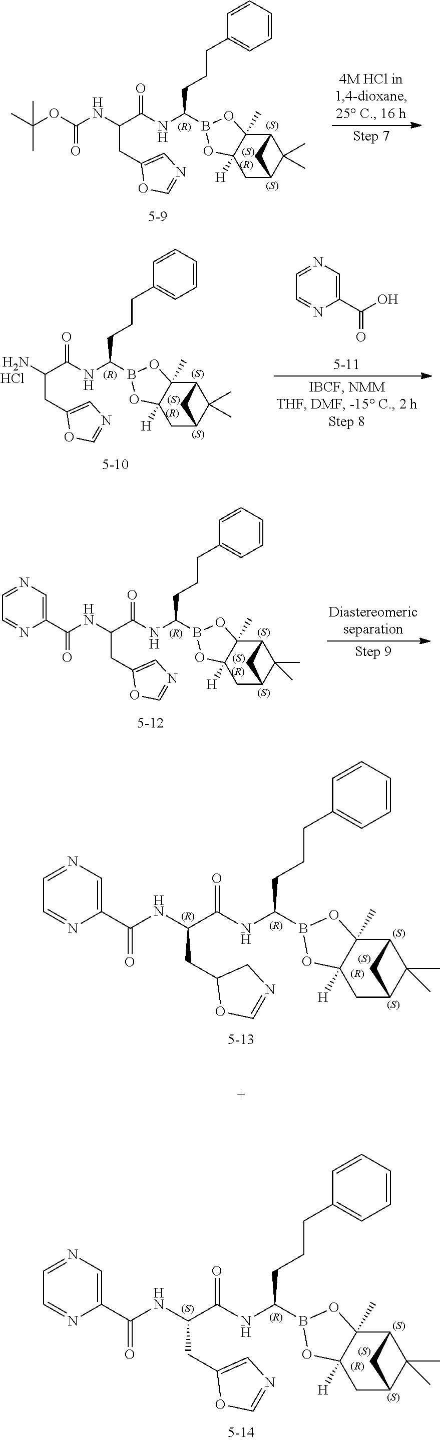

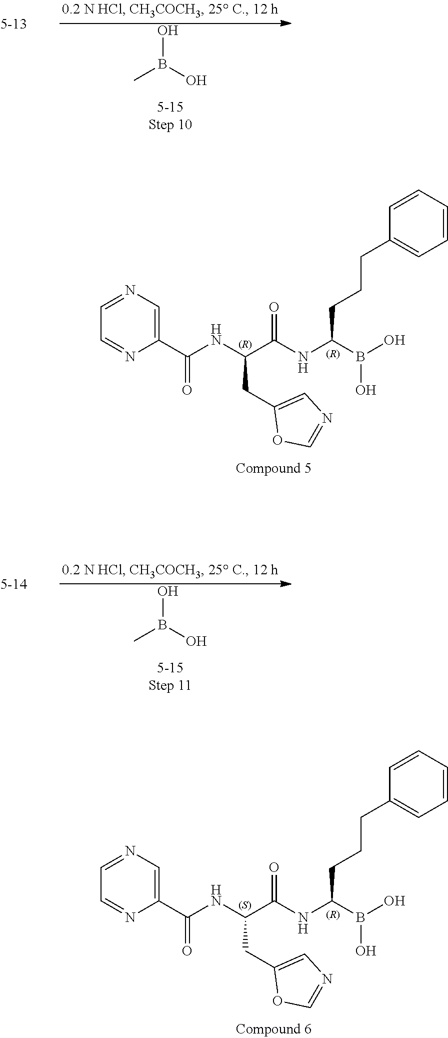

Synthesis of oxazol-5-ylmethanol [Step 1]: To a stirred solution of propyl_oxazole-5-carboxylate (5-1, 4 g, 25.8 mmol) in methanol (97 mL) and dry THF (46.4 mL) was added UCl (6.5 g, 155 mmol) at −10° C. NaBH4 (5.9 g, 155 mmol) was added to it. The reaction mixture was allowed to stirred for 16 h at 25° C. Reaction was monitored by TLC. The reaction mass was partitioned between saturated aqueous sodium potassium tertarate and EtOAc. Organic layer was collected. Aqueous layer was further extracted with EtOAc (twice). Combined organic layer was washed with brine, dried over Na2SO4 and concentrated under reduced pressure. The product was purified through flash chromatography to get oxazol-5-yI methanol (5-2, 2 g). ¹H NMR (400 MHz, DMSO-d₆) δ: 8.27 (s, 1H), 7.03 (s, 1H), 5.39 (s, 1H), 4.45 (d, 2H).

Synthesis of oxazole-5-carbaldehyde [Step 2]: To a stirred solution of oxalyl chloride (2.1 mL, 24.2 mmol) in DCM (47 mL) was added DMSO (4.3 mL, 61 mmol) at −78° C. and stirred for 30 min. Oxazol-5-ylmethanol (5-2, 1.2 g, 12.1 mmol) was added and stirred at −78° C. for 30 min. TEA (6.6 mL, 49 mmol) was added at −78° C. and stirred at same temperature for 30 min and then at 0° C. for 1 h. The progress of the reaction was monitored by TLC. The reaction mixture was diluted with water and extracted with DCM (twice). Combined organic layer was dried over Na₂SO₄ and concentrated under reduced pressure. The product was purified by combi-flash column chromatography to get oxazole-5-carbaldehyde (5-3, 800 mg). ¹H NMR (400 MHz, DMSO-d₆) δ: 9.81 (s, 1H), 8.77 (s, 1H), 8.21 (s, 1H).

Synthesis of methyl (E)-2-((tert-butoxycarbonyl)amino)-3-(oxazol-5-yl)acrylate [Step 3]: To a stirred solution of methyl 2-(tert-butoxycarbonylamino)-2-dimethoxyphosphoryl-acetate (5-4, 1.99 g, 6.70 mmol) and oxazole-5-carbaldehyde (5-3, 500 mg, 5.15 mmol) in THF (4 mL) was added Tetramethylguanidin (2.1 mL, 16.5 mmol) at ice-cold condition and stirred for 2 h at 25° C.

Volatiles were removed under reduced pressure and the residue was partitioned between water and ethyl acetate. Organic layer was washed with saturated aqueous NH₄Cl, dried over Na₂SO₄ and concentrated under reduced pressure. The product was purified by flash column chromatography to get methyl (E)-2-(tert-butoxycarbonylamino)-3-oxazol-5-yl-prop-2-enoate (5-5, 500 mg). [M+H]⁺=269; ¹H NMR (400 MHz, DMSO-d₆) δ: 8.76 (s, 1H), 8.52 (s, 1H), 7.49 (s, 1H), 7.04 (s, 1H), 3.73 (s, 3H), 1.39 (s, 9H).

Synthesis of (methyl 2-((tert-butoxycarbonyl)amino)-3-(oxazol-5-yl)propanoate [Step 4]: To a stirred solution of methyl (E)-2-(tert-butoxycarbonylamino)-3-oxazol-5-yl-prop-2-enoate (5-5, 400 mg, 1.5 mmol) in Methanol (10 mL) was added 10% Pd/C (100 mg) and hydrogenated under H₂ balloon pressure for 16 h at 25° C. The reaction was monitored by TLC. The reaction mixture was filtered through celite bed. Filtrate liquid was concentrated under reduced pressure to get methyl 2-(tert-butoxycarbonylamino)-3-oxazol-5-yl-propanoate (5-6, 350 mg). [M+H]⁺=271; ¹H NMR (400 MHz, DMSO-d₆) δ: 8.23 (s, 1H), 7.36 (d, 1H), 6.89 (s, 1H), 4.27-4.26 (m, 1H), 3.62 (bs, 3H), 3.15-2.98 (m, 2H), 1.34 (bs, 9H).

Synthesis of 2-((tert-butoxycarbonyl)amino)-3-(oxazol-5-yl)propanoic acid [Step 5]: To a stirred solution of methyl 2-(tert-butoxycarbonylamino)-3-oxazol-5-yl-propanoate (5-6, 400 mg, 1.5 mmol) in THF (10 mL) was added a solution of LiOH (93 mg, 2.2 mmol) in water (2 mL) and stirred for 16 h at 25° C. The reaction was monitored by LCMS. After completion the reaction mixture was concentrated and diluted with 10 mL water. The aqueous part was washed with 10 mL EtOAc. The residual aqueous part was acidified with NaHSO4 and extracted with 10% (MeOH/DCM). Organic layer was concentrated to get 2-(tert-butoxycarbonylamino)-3-oxazol-5-yl-propanoic acid (5-7, 180 mg). [M−H]⁻=255; ¹H NMR (400 MHz, DMSO): δ 8.22 (s, 1H), 7.12 (d, 1H), 6.87 (s, 1H), 4.14-4.12 (m, 1H), 3.13-3.08 (m, 1H), 3.00-2.94 (m, 1H), 1.37-1.34 (m, 9H).

Synthesis of tort-butyl (3-(oxazol-5-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)carbamate [Step 6]: To a stirred solution of 2-((tert-butoxycarbonyl)amino)-3-(oxazol-5-yl)propanoic acid (79-7, 232 mg, 0.9 mmol) in THF (8 mL) was added IBCF (0.12 mL, 0.9 mmol) followed by NMM (0.12 mL, 0.9 mmol) at −15° C. and stirred for 30 min. A solution of (R)-4-phenyl-1-((3aS, 4S, 6S, 7aR)-3a, 5, 5-trimethylhexahydro-4, 6-methanobenzo[d][1, 3, 2]dioxaborol-2-yl)butan-1-amine hydrochloride (5-8, 300 mg, 0.8 mmol) in DMF (1 mL) was added to it followed by NMM (0.1 mL, 0.8 mmol) at −15° C. It was gradually warmed to 0° C. and stirred for 2 h. LCMS of crude reaction mass confirmed the formation of desired product. It was neutralized with saturated aqueous 0.1 N HCl solution and extracted with ethyl acetate. Combined organic layer was washed with 5% K₂CO₃ solution, water and brine, dried over Na₂SO₄ and evaporated under reduced pressure to get tert-butyl (3-(oxazol-5-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)carbamate (5-9, 450 mg). [M−H]⁻=564.

Synthesis of 2-amino-3-(oxazol-5-yl)-N—((R)-4-phenyl-1-((3aS, 4S, 6S, 7aR)-3a, 5, 5-trimethylhexahydro-4, 6-methanobenzo[d][1, 3, 2]dioxaborol-2-yl)butyl)propanamide hydrochloride [Step 7]: To a solution of tert-butyl (3-(oxazol-5-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)carbamate (5-9, 450 mg, 0.8 mmol) in 1, 4-dioxane (6 mL) was added 4 M HCl in 1, 4-dioxane (5 mL) at 0° C. It was gradually warmed to 25° C. and stirred for 16 h. The reaction was monitored by TLC. Volatiles were removed under reduced pressure to get 2-amino-3-(oxazol-5-yl)-N—((R)-4-phenyl-1-((3aS, 4S, 6S, 7aR)-3a, 5, 5-trimethylhexahydro-4, 6-methanobenzo[d][1, 3, 2]dioxaborol-2-yl)butyl)propanamide hydrochloride (5-10, 400 mg). [M−H]⁻=464.

Synthesis of N-(3-(oxazol-5-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide [Step 8]: To a stirred solution of pyrazine-2-carboxylic acid (5-11, 110 mg, 0.9 mmol) in THF (8 mL) was added IBCF (0.12 mL, 0.9 mmol) and NMM (0.12 mL, 0.9 mmol) at −15° C. and stirred for 30 min. A solution of 2-amino-3-(oxazol-5-yl)-N—((R)-4-phenyl-1-((3aS, 4S, 6S, 7aR)-3a, 5, 5-trimethylhexahydro-4, 6-methanobenzo[d][1, 3, 2]dioxaborol-2-yl)butyl)propanamide hydrochloride (5-10, 400 mg, 0.8 mmol) in DMF (1 mL) was added to it followed by NMM (0.1 mL, 0.8 mmol). It was gradually warmed to 0° C. and stirred for 2 h. The reaction was monitored by LCMS. It was neutralized with saturated aqueous 0.1 N HCl solution and extracted with ethyl acetate (thrice). The combined organic layer was washed with 5% K₂CO₃ solution, water and brine, dried over Na₂SO₄ and evaporated under reduced pressure to get N-(3-(oxazol-5-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide (5-12, 450 mg).

Synthesis of N—((R)-3-(oxazol-5-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide and N—((S)-3-(oxazol-5-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide [Step 9]: N-(3-(oxazol-5-yl)-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)propan-2-yl)pyrazine-2-carboxamide (5-12, 450 mg) was purified via SFC chiral separation and lyophilized to afford peak 1 as ((R)-3-(oxazol-5-yl)-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)propan-2-yl)pyrazine-2-carboxamide (5-13, 65 mg). LCMS (ESI) Calcd. for C₃₁H₃₈BN₅O₅: 571, found [M−H]⁻=570. Peak 2 was isolated as N—((S)-3-(oxazol-5-yl)-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)propan-2-yl)pyrazine-2-carboxamide (5-14, 65 mg). [M−H]⁻=570. The absolute stereochemistry of these products was not determined.

Prep SFC method: Column was C-AMYLOSE-A (30 mm×250 mm), 5μ, flow rate at 60 g/min, mobile phase: 70% CO2+30% (MeOH), ABPR: 100 bar, temp: 35° C., UV: 268 nm, Diluent: MeOH+EtOH+MeCN, Sample concentration: 63.5 mg/ml, loading: 95.2 mg/12.2 min.

Synthesis of ((R)-1-((R)-3-(oxazol-5-yl)-2-(pyrazine-2-carboxamido)propanamido)-4-phenylbutyl)boronic acid (Compound 5) [Step 10]: To a stirred solution of N—((R)-3-(oxazol-5-yl)-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)propan-2-yl)pyrazine-2-carboxamide (5-13, 75 mg, 0.1 mmol) and methylboronic acid (118 mg, 1.9 mmol) in acetone (4.0 mL) was added 0.2 N HCl (4.0 mL) and stirred at 25° C. for 16 h. The reaction was monitored by LCMS. The volatiles were evaporated under reduced pressure and purified by prep HPLC purification and lyophilized to afford ((R)-1-((R)-3-(oxazol-5-yl)-2-(pyrazine-2-carboxamido)propanamido)-4-phenylbutyl) boronic acid (Compound 5, 26 mg). [M−H]⁻=436; ¹H NMR (400 MHz, DMSO-d₆+2 drops D₂O): δ 9.15 (s, 1H), 8.89 (s, 1H), 8.74 (s, 1H), 8.15 (s, 1H), 7.21-7.11 (m, 5H), 6.84 (s, 1H), 4.80-4.77 (m, 1H), 3.34-3.05 (m, 3H), 1.50 (bs, 4H). The absolute stereochemistry of this product was not determined.

Synthesis of (R)-1-((S)-3-(oxazol-5-yl)-2-(pyrazine-2-carboxamido)propanamido)-4-phenyl butyl)boronic acid (Compound 6) [Step 11]: To a stirred solution of N-[(1S)-1-(oxazol-5-ylmethyl)-2-oxo-2-[[(1R)-4-phenyl-1-[(1S,2S,6R,8S)-2,9,9-trimethyl-3,5-dioxa-4-boratricyclo [6.1.1.02,6]decan-4yl]butyl]amino]ethyl]pyrazine-2-carboxamide (5-14, 75 mg, 0.1 mmol) and methylboronic acid (118 mg, 1.9 mmol) in acetone (4 mL) was added 0.2 N_HCl (4 mL) and stirred at 25° C. for 16h. Reaction was monitored by LCMS. The volatiles were evaporated and the residue was purified by prep HPLC purification and lyophilized to afford [(1R)-1-[[(2S)-3-oxazol-5-yl-2-(pyrazine-2-carbonylamino) propanoyl]amino]-4-phenyl-butyl]boronic acid (Compound 6, 26 mg). [M−H]⁻=436; ¹H NMR (400 MHz, DMSO-d₆+2 drops D₂O): δ 9.16 (s, 1H), 8.89 (s, 1H), 8.75 (s, 1H), 8.14 (s, 1H), 7.26-7.22 (m, 2H), 7.15-7.14 (m, 3H), 6.82 (s, 1H), 4.82-4.79 (m, 1H), 3.37-3.13 (m, 3H), 1.49 (bs, 4H). The absolute stereochemistry of this product was not determined.

### Example 7

### Synthesis of ((R)-1-((R)-3-(1H-indol-3-yl)-2-(pyrazine-2-carboxamido)propanamido)-4-phenylbutyl)boronic acid

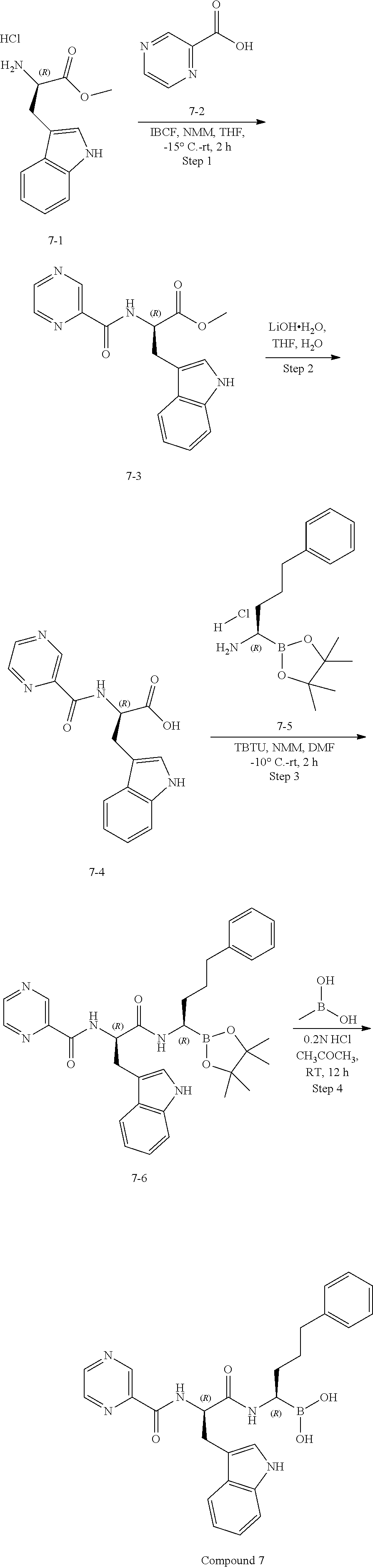

Synthesis of methyl (pyrazine-2-carbonyl)-D-tryptophanate [Step 1]: To a stirred solution of pyrazine-2-carboxylic acid (7-2, 509 mg, 4.1 mmol) in THF (10 mL) was added IBCF (0.4 mL, 3.4 mmol) and NMM (0.45 mL, 4.1 mmol) dropwise at −15° C. and stirred at same temperature for 30 min. Methyl D-tryptophanate hydrochloride (7-1, 950 mg, 3.7 mmol) followed by NMM (0.4 mL, 3.7 mmol) was added to the reaction mixture under same condition and gradually warmed to 0° C. and stirred for 2 h. LCMS of crude reaction mass confirmed the formation of desired product. It was neutralized with saturated aqueous 0.1 N HCl solution and extracted with ethyl acetate. Combined organic layer was washed with 5% K₂CO₃ solution, water, brine, dried over Na₂SO₄ and evaporated under reduced pressure. The product was purified through flash column chromatography to afford methyl (pyrazine-2-carbonyl)-D-tryptophanate (7-3, 1.0 g). [M−H]⁻=323.

Synthesis of ((pyrazine-2-carbonyl)-D-tryptophan [Step 2]: To a stirred solution of methyl (pyrazine-2-carbonyl)-D-tryptophanate (7-3, 1.0 g, 3.1 mmol) in THF (6 mL) was added a solution of LiOH·H2O (388 mg, 9.3 mmol) in Water (2 mL) at 0° C. and stirred at RT for 2 h. the reaction was monitored by LCMS. The reaction mixture was neutralized with 1N HCl (pH: 5-6) and lyophilized to get (pyrazine-2-carbonyl)-D-tryptophan (7-4, 950 mg). [M+H]⁺=311.

Synthesis of N—((R)-3-(1H-Indol-3-yl)-1-oxo-1-(((R)-4-phenyl-1-(4, 4, 5, 5-tetramethyl-1, 3, 2-dioxaborolan-2-yl)butyl)amino)propan-2-yl)pyrazine-2-carboxamide [Step 3]: To a stirred solution of (1R)-4-phenyl-1-(4, 4, 5, 5-tetramethyl-1, 3, 2-dioxaborolan-2-yl)butan-1-amine hydrochloride (7-5, 301 mg, 0.9 mmol) in DMF (3 mL) was added (pyrazine-2-carbonyl)-D-tryptophan (7-4, 300 mg, 0.9 mmol) followed by NMM (0.5 mL, 4.8 mmol) at −10° C. TBTU (341 mg, 1.06 mmol) was added portion wise and stirred for 1.5 h at ambient temperature. The reaction was monitored by LCMS. Reaction mixture was partitioned between EtOAc and water. Organic layer was collected and washed with water (thrice) and brine, dried over anhydrous Na₂SO₄ and evaporated under reduced pressure to get N-[(1R)-1-(1H-indol-3-ylmethyl)-2-oxo-2-[[(1R)-4-phenyl-1-(4, 4, 5, 5-tetramethyl-1, 3, 2-dioxaborolan-2-yl) butyl]amino]ethyl]pyrazine-2-carboxamide (7-6, 500 mg). [M−H]⁻=567.

Synthesis of ((R)-1-((R)-3-(1H-indol-3-yl)-2-(pyrazine-2-carboxamido)propanamido)-4-phenylbutyl)boronic acid [Step 4]: To a stirred solution of N-[(1R)-1-(1H-indol-3-ylmethyl)-2-oxo-2-[[(1R)-4-phenyl-1-(4, 4, 5, 5-tetramethyl-1, 3, 2-dioxaborolan-2-yl)butyl]amino]ethyl]pyrazine-2-carboxamide (7-6, 600 mg, 1.1 mmol) and methylboronic acid (633 mg, 10.6 mmol) in acetone (10 mL) was added 0.2 N HCl (10 mL) and stirred at RT for overnight. The reaction was monitored by LCMS. The volatiles were evaporated and purified via prep HPLC purification and lyophilized to afford ((R)-1-((R)-3-(1H-indol-3-yl)-2-(pyrazine-2-carboxamido) propanamido)-4-phenylbutyl) boronic acid (Compound 7.57 mg). [M−H]⁻=484; ¹H NMR (400 MHz, DMSO-d₆+2 drops D20) δ: 9.10 (s, 1H), 8.83 (s, 1H), 8.67 (s, 1H), 7.55-7.53 (m, 1H), 7.29-7.27 (m, 1H), 7.22-7.18 (m, 2H), 7.13-7.07 (m, 4H), 7.04-6.99 (m, 1H), 6.91-6.87 (m, 1H), 4.76 (t, 1H), 3.24-3.14 (m, 2H), 3.06 (br s, 1H), 2.54 (br s, 2H), 1.41 (br s, 4H).

### Example 8 and Example 9

### Synthesis of ((R)-3-methyl-1-((R)-3-(1-methyl-1H-Imidazol-2-yl)-2-(pyrazine-2-carboxamido)propanamido)butyl)boronic acid (Compound 8) and ((R)-3-methyl-1-((S)-3-(1-methyl-1H-imidazol-2-yl)-2-(pyrazine-2-carboxamido)propanamido)butyl)boronic acid (Compound 9)

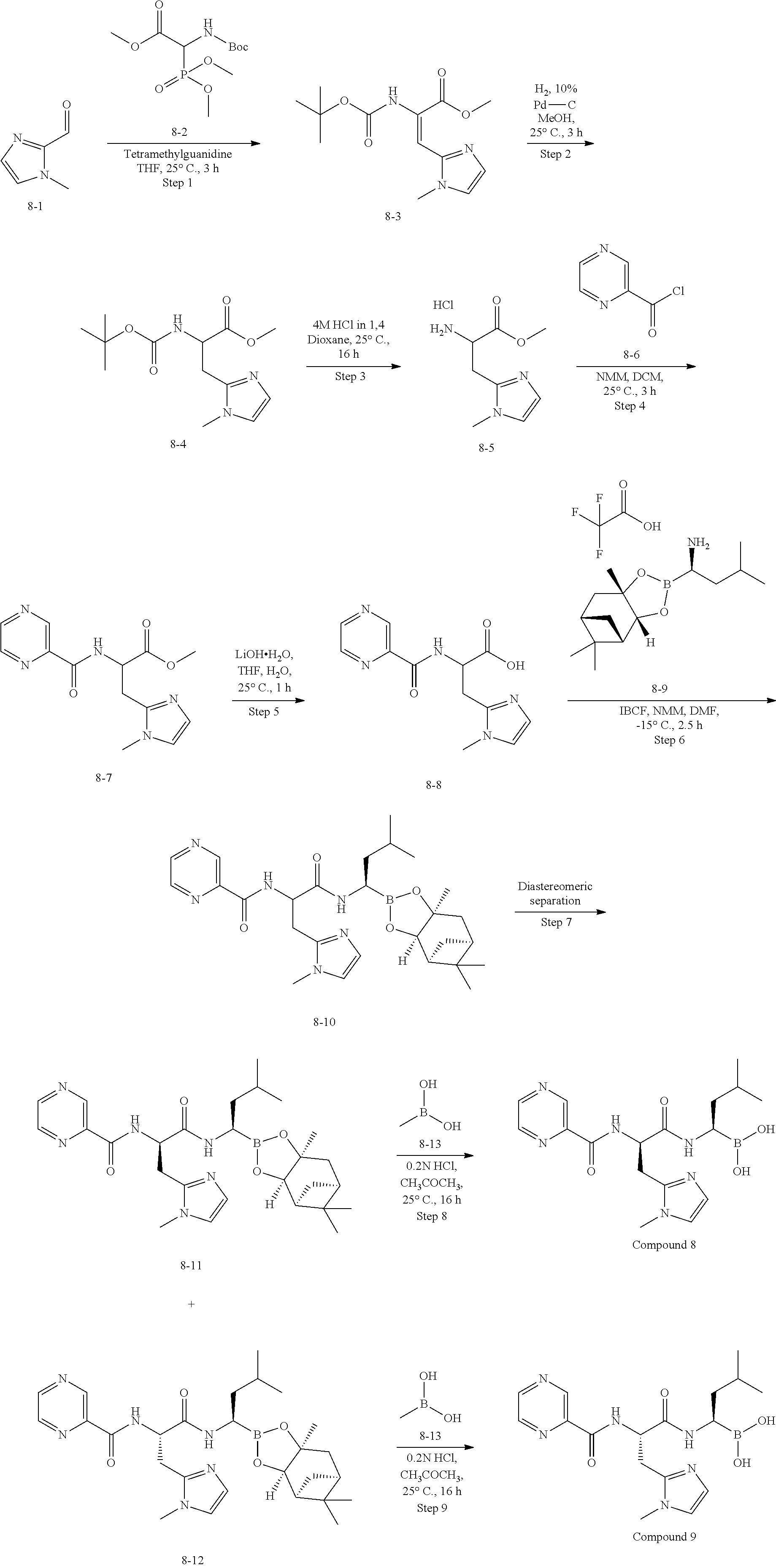

Synthesis of methyl (E)-2-((tert-butoxycarbonyl)amino)-3-(1-methyl-1H-imidazol-2-yl)acrylate [Step 1]: 1, 1, 3, 3-tetramethylguanidine (1.3 mL, 9.9 mmol) was added dropwise over 5 mins at 0° C. to a stirred solution 1-methyl-1H-imidazole-2-carbaldehyde (8-1, 1 g, 9.1 mmol) and methyl 2-((tert-butoxycarbonyl)amino)-2-(dimethoxyphosphoryl)acetate (8-2, 2.6 g, 8.8 mmol) in THF (10 mL) and stirred at 25° C. for 3 h. The reaction was monitored by TLC. Volatiles were removed under reduced pressure and the residue was partitioned between water and ethyl acetate. The organic layer was washed with saturated aqueous NH₄Cl solution, dried over Na₂SO₄ and concentrated under reduced pressure to afford methyl (E)-2-((tert-butoxycarbonyl)amino)-3-(1-methyl-1H-imidazol-2-yl)acrylate (8-3, 1.4 g). [M+H]⁺=282.

Synthesis of methyl 2-((tert-butoxycarbonyl)amino)-3-(1-methyl-1H-imidazol-2-yl)propanoate [Step 2]: To a stirred solution of methyl (E)-2-((tert-butoxycarbonyl)amino)-3-(1-methyl-1H-imidazol-2-yl)acrylate (8-3, 1.4 g, 4.9 mmol) in methanol (20 mL) was added 10% Pd/C (300 mg) and hydrogenated under H₂ balloon pressure at 25° C. for 2 h. The reaction was monitored by TLC. The reaction mixture was filtered through a pad of celite, the pad was washed with methanol. Combined filtrate liquid was evaporated under reduced pressure to get methyl 2-((tert-butoxycarbonyl)amino)-3-(1-methyl-1H-imidazol-2-yl)propanoate (8-4, 1.2 g). [M+H]⁺=284.

Synthesis of methyl 2-amino-3-(1-methyl-1H-Imidazol-2-yl)propanoate hydrochloride [Step 3]: To a stirred solution of methyl 2-((tert-butoxycarbonyl)amino)-3-(1-methyl-1H-imidazol-2-yl)propanoate (8-4, 1 g, 3.5 mmol) in 1, 4-dioxane (5 mL) was added 4M HCl in 1, 4-dioxane (8.8 mL, 35.3 mmol) at 0° C. and stirred at 25° C. for 16 h. The reaction was monitored by TLC. The reaction mixture was concentrated under reduced pressure to get methyl 2-amino-3-(1-methyl-1H-imidazol-2-yl)propanoate hydrochloride (8-5, 700 mg). [M+H]⁺=184.

Synthesis of methyl 3-(1-methyl-1H-Imidazol-2-yl)-2-(pyrazine-2-carboxamido) propanoate [Step 4]: To a stirred solution of methyl 2-amino-3-(1-methyl-1H-imidazol-2-yl)propanoate hydrochloride (8-5, 700 mg, 3.1 mmol) in DCM (7 mL), NMM (1 mL, 7.3 mmol) was added dropwise at ice-cold condition and stirred for 30 min. A solution of pyrazine-2-carbonyl chloride (8-6, 543 mg, 3.8 mmol) in DCM (3 mL) was added dropwise to the reaction mixture at ice-cold condition and stirred for 2 h. The reaction was monitored by TLC. The reaction mixture was purified by flash chromatography to afford methyl 3-(1-methyl-1H-imidazol-2-yl)-2-(pyrazine-2-carboxamido)propanoate (8-7, 350 mg). [M+H]⁺=290.

Synthesis of 3-(1-methyl-1H-Imidazol-2-yl)-2-(pyrazine-2-carboxamido)propanoic acid [Step 5]: To a stirred solution of methyl 3-(1-methyl-1H-imidazol-2-yl)-2-(pyrazine-2-carboxamido)propanoate (8-7, 250 mg, 1.2 mmol) in THF (10 mL) and water (2 mL) was added LiOH·H2O (63 mg, 1.5 mmol) and stirred at room temperature for 1 h. Volatiles were removed under reduced pressure and diluted with water. It was acidified with 0.2N HCl (pH: 3) and lyophilized to obtain 3-(1-methyl-1H-imidazol-2-yl)-2-(pyrazine-2-carboxamido) propanoic acid (8-8, 220 mg). [M+H]⁺=276.

Synthesis of N-(1-(((R)-3-methyl-1-((3aR, 4S, 6S, 7aS)-5, 5, 7a-trimethylhexahydro-4, 6-methanobenzo[d][1, 3, 2]dioxaborol-2-yl)butyl)amino)-3-(1-methyl-1H-Imidazol-2-yl)-1-oxopropan-2-yl)pyrazine-2-carboxamide [Step 6]: To a stirred solution of 3-(1-methyl-1H-imidazol-2-yl)-2-(pyrazine-2-carboxamido)propanoic acid (8-8, 152 mg, 0.5 mmol) in DMF (2 mL) was added IBCF (0.06 mL, 0.5 mmol) followed by NMM (0.07 mL, 0.5 mmol) at −15° C. and stirred for 45 min. A solution of (1R)-3-methyl-1-[(1S, 2R, 6S, 8S)-6, 9, 9-trimethyl-3, 5-dioxa-4-boratricyclo[6.1.1.02, 6]decan-4-yl]butan-1-amine; 2, 2, 2-trifluoroacetic acid (8-9, 190 mg, 0.5 mmol) in DMF (2 mL) was added dropwise followed by NMM (0.06 mL, 0.5 mmol) and stirred at −15° C. for 1 h. The reaction was monitored by LCMS. The reaction mixture was diluted with EtOAc, washed with 0.1 N HCl, 5% aq. K₂CO₃, water and brine, dried over anhy. Na2SO4 and concentrated under reduced pressure to get N-(1-(((R)-3-methyl-1-((3aR, 4S, 6S, 7aS)-5, 5, 7a-trimethylhexahydro-4, 6-methanobenzo[d][1, 3, 2]dioxaborol-2-yl)butyl)amino)-3-(1-methyl-1H-imidazol-2-yl)-1-oxopropan-2-yl)pyrazine-2-carboxamide (8-10, 100 mg). [M−H]⁻=522.

Isolation of N—((R)-1-(((R)-3-methyl-1-((3aR,4S,6S,7aS)-5,5,7a-trimethylhexahydro-4,6-methanobenzo[d][1,3,2]dioxaborol-2-yl)butyl)amino)-3-(1-methyl-1H-imidazol-2-yl)-1-oxopropan-2-yl)pyrazine-2-carboxamide (8-11) and N—((S)-1-(((R)-3-methyl-1-((3aR,4S,6S,7aS)-5,5,7a-trimethylhexahydro-4,6-methanobenzo[d][1,3,2]dioxaborol-2-yl)butyl)amino)-3-(1-methyl-1H-Imidazol-2-yl)-1-oxopropan-2-yl)pyrazine-2-carboxamide (8-12) [Step 7]: N-(1-(((R)-3-methyl-1-((3aR,4S,6S,7aS)-5,5,7a-trimethylhexahydro-4,6-methanobenzo[d][1,3,2]dioxaborol-2-yl)butyl)amino)-3-(1-methyl-1H-imidazol-2-yl)-1-oxopropan-2-yl)pyrazine-2-carboxamide (8-10, 100 mg, 1.0 mmol) was separated via SFC chiral and lyophilized to afford Peak 1 as N—((R)-1-(((R)-3-methyl-1-((3aR,4S,6S,7aS)-5,5,7a-trimethylhexahydro-4,6-methanobenzo[d][1,3,2]dioxaborol-2-yl) butyl)amino)-3-(1-methyl-1H-imidazol-2-yl)-1-oxopropan-2-yl)pyrazine-2-carboxamide (8-11, 38 mg) and Peak 2 as N—((S)-1-(((R)-3-methyl-1-((3aR,4S,6S,7aS)-5,5,7a-trimethylhexahydro-4,6-methanobenzo[d][1,3,2]dioxaborol-2-yl)butyl)amino)-3-(1-methyl-1H-imidazol-2-yl)-1-oxopropan-2-yl)pyrazine-2-carboxamide (85-12, 33 mg). The absolute stereochemistry of the compounds was not determined.

SFC purification method: Chiral separation was performed on a Thar SFC-80 series instrument using C-Amylose A column (30 mm×250 mm), Sp, operating at 35° C. temperature, maintaining flow rate of 40 gm/min, using 80% CO₂ in super critical state and 20% methanol as the mobile phase, run isocratic for up to 13 min and also isobaric conditions of 100 bar at a detection of 230 nm wavelength.

Synthesis of ((R)-3-methyl-1-((R)-3-(1-methyl-1H-imidazol-2-yl)-2-(pyrazine-2-carboxamido)propanamido)butyl)boronic acid (Compound 8) [Step 8]: To a solution of N-(1-(((R)-3-methyl-1-((3aR, 4S, 6S, 7aS)-5, 5, 7a-trimethylhexahydro-4, 6-methanobenzo[d][1, 3, 2]dioxaborol-2-yl)butyl)amino)-3-(1-methyl-1H-imidazol-2-yl)-1-oxopropan-2-yl)pyrazine-2-carboxamide (8-11, 35 mg, 0.06 mmol) and methylboronic acid (8-13, 48 mg, 0.8 mmol) in acetone (2 mL) was added 0.2N aq. HCl (2 mL) at 25° C. and stirred at RT for 16 h. Volatiles were removed under reduced pressure and purified by prep HPLC purification and lyophilized to afford (3-methyl-1-((R)-3-(1-methyl-1H-imidazol-2-yl)-2-(pyrazine-2-carboxamido) propanamido)butyl)boronic acid (Compound 8, 12 mg). [M−H]⁻=387; ¹H NMR (400 MHz, MeOD): δ 9.18 (s, 1H), 8.82 (s, 1H), 8.70 (s, 1H), 7.46 (d, 2H), 5.21 (t, 1H), 3.93 (s, 3H), 3.75-3.69 (m, 1H), 3.50-3.44 (m, 1H), 2.96 (t, 1H), 1.59-1.56 (m, 1H), 1.37-1.27 (m, 2H), 0.89 (s, 6H). The absolute stereochemistry of the compound was not determined.

Synthesis of ((R)-3-methyl-1-((S)-3-(1-methyl-H-imidazol-2-yl)-2-(pyrazine-2-carboxamido) propanamido)butyl)boronic acid (Example 9) [Step 9]: To a solution of N-(1-(((R)-3-methyl-1-((3aR, 4S, 6S, 7aS)-5, 5, 7a-trimethylhexahydro-4, 6-methanobenzo[d][1, 3, 2]dioxaborol-2-yl)butyl)amino)-3-(1-methyl-1H-imidazol-2-yl)-1-oxopropan-2-yl)pyrazine-2-carboxamide (8-12, 30 mg, 0.05 mmol) and methylboronic acid (8-13, 41 mg, 0.7 mmol) in acetone (2 mL) was added 0.2N aq. HCl (2 mL) at 25° C. and stirred at RT for 16 h. Volatiles were removed under reduced pressure and purified by prep HPLC purification and lyophilized to afford ((R)-3-methyl-1-((S)-3-(1-methyl-1H-imidazol-2-yl)-2-(pyrazine-2-carboxamido) propanamido)butyl)boronic acid (Compound 9, 9 mg). [M−H]⁻=387; ¹H NMR (400 MHz, MeOD): δ 9.18 (s, 1H), 8.82 (s, 1H), 8.71 (s, 1H), 7.45 (d, 2H), 5.29-5.27 (m, 1H), 3.92 (s, 3H), 3.76-3.71 (m, 1H), 3.49-3.43 (m, 1H), 2.93-2.90 (m, 1H), 1.59-1.56 (m, 1H), 1.40-1.36 (m, 2H), 0.90 (s, 6H). The absolute stereochemistry of the compounds was not determined.

### Example 10 and Example 11

### Synthesis of ((R)-1-((R)-3-(oxazol-2-yl)-2-(pyrazine-2-carboxamido)propanamido)-4-phenyl butyl)boronic acid (Compound 10) and ((R)-1-((S)-3-(oxazol-2-yl)-2-(pyrazine-2-carboxamido)propanamido)-4-phenylbutyl)boronic acid (Compound 11)

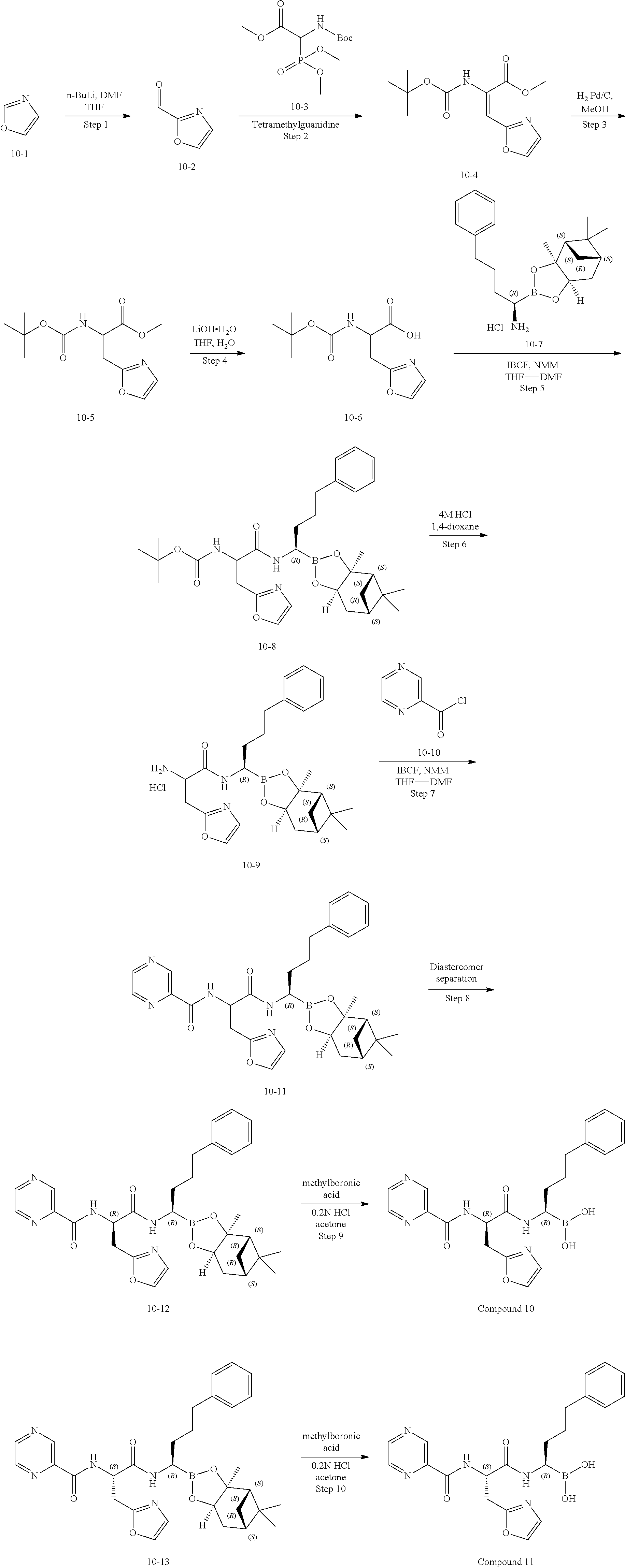

Synthesis of oxazole-2-carbaldehyde [Step 1]: To a solution of oxazole (10-1, 1.0 mL, 15.2 mmol) in THF (40 mL) was added n-BuLi (2.5 M in hexanes) (6.1 mL, 15.2 mmol) at −78° C. After 30 min, a solution of N,N-dimethylformamide (1.2 mL, 15.2 mmol) in THF (5 mL) was added dropwise, and the reaction mixture was allowed to warm to 25° C. After 16 h, the reaction was quenched with water, and extracted with Et₂O (twice). Combined organic phase was washed with brine, dried over anhydrous Na₂SO₄, filtered and concentrated under reduced pressure to afford oxazole-2-carbaldehyde (10-2, 200 mg), which was used in the next step without further purification. ¹H NMR (400 MHz, CDCl₃): δ 9.79 (s, 1H), 7.89 (s, 1H), 7.44 (s, 1H).

Synthesis of methyl (E)-2-((tert-butoxycarbonyl)amino)-3-(oxazol-2-yl)acrylate [Step 2]: 1, 1, 3, 3-tetramethylguanidine (1.4 mL, 11.3 mmol) was added dropwise over one minute to a stirred solution of oxazole-2-carbaldehyde (10-2, 1.0 g, 10.3 mmol) and methyl 2-((tert-butoxycarbonyl)amino)-2-(dimethoxyphosphoryl)acetate (10-3, 3.0 g, 10.0 mmol) in THF (10 mL). The mixture was stirred for 2 h at 25° C. The reaction mixture was concentrated under reduced pressure, and partitioned between water and EtOAc. The organic phase was washed with brine, dried over anhydrous Na2SO4, filtered and concentrated under reduced pressure. The compound was purified by column chromatography methyl (E)-2-((tert-butoxycarbonyl)amino)-3-(oxazol-2-yl)acrylate (10-4, 300 mg). [M+H]⁺=269.

Synthesis of methyl 2-((tert-butoxycarbonyl)amino)-3-(oxazol-2-yl)propanoate [Step 3]: To a solution of methyl (E)-2-((tert-butoxycarbonyl)amino)-3-(oxazol-2-yl)acrylate (10-4, 700 mg, 2.6 mmol) in methanol (25 mL) was added 10% Pd/C (70 mg, 10 wt %) under N₂. The reaction vessel was evacuated and backfilled with H₂ (twice), and then kept under a positive pressure of H₂. After stirring for 16 h at 25° C., the reaction mixture was filtered through a pad of celite. The filtrate was concentrated under reduced pressure to get methyl 2-((tert-butoxycarbonyl)amino)-3-(oxazol-2-yl)propanoate (10-5, 650 mg), which was used for the next step without further purification. [M+H]⁺=271.

Synthesis of 2-((fort-butoxycarbonyl)amino)-3-(oxazol-2-yl)propanoic acid [Step 4]: To a solution of methyl 2-((tert-butoxycarbonyl)amino)-3-(oxazol-2-yl)propanoate (10-5, 650 mg, 2.4 mmol) in THF (10 mL)-water (2 mL) was added LiOH·H₂O (152 mg, 3.6 mmol). The reaction mixture was stirred at ambient temperature. After 2 h, the reaction mixture was concentrated under reduced pressure, and diluted with 10 ml water. The aqueous phase was washed with EtOAc. The aqueous phase was acidified with 1N aqueous HCl (till pH 2), and lyophilized to get 2-((tert-butoxycarbonyl)amino)-3-(oxazol-2-yl)propanoic acid (10-6, 550 mg), which was forwarded to the next step without further purification. [M+H]⁺=257.

Synthesis of tort-butyl (3-(oxazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)carbamate [Step 5]: To a solution of 2-((tert-butoxycarbonyl)amino)-3-(oxazol-2-yl)propanoic acid (10-6, 465 mg, 1.8 mmol) in THF (10 mL) was added IBCF (0.2 mL, 1.8 mmol) followed by NMM (0.2 mL, 1.8 mmol) at −15° C. After 45 min, a solution of (R)-4-phenyl-1-((3aS, 4S, 6S, 7aR)-3a, 5, 5-trimethylhexahydro-4, 6-methanobenzo[d][1, 3, 2]dioxaborol-2-yl)butan-1-amine hydrochloride (10-7, 600 mg, 1.6 mmol) in DMF (2 mL) was added dropwise followed by NMM (0.2 mL, 1.6 mmol). After stirring for 1 h at the same temperature, the reaction mixture was diluted with EtOAc, and washed successively with 0.1 N aqueous HCl (twice), 5% aqueous K₂CO₃ (twice), water (twice) and brine (twice). The organic phase was dried over anhydrous Na2SO4, filtered and concentrated under reduced pressure to afford tert-butyl (3-(oxazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)carbamate (10-8, 700 mg), which was used in the next step without further purification. [M−H]⁻=564.

Synthesis of 2-amino-3-(oxazol-2-yl)-N—((R)-4-phenyl-1-((3aS, 4S, 6S, 7aR)-3a, 5, 5-trimethylhexahydro-4, 6-methanobenzo[d][1, 3, 2]dioxaborol-2-yl)butyl)propanamide hydrochloride [Step 6]: To a solution of tert-butyl (3-(oxazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)carbamate (10-8, 700 mg, 1.24 mmol) in 1, 4-dioxane (5 mL) was added HCl (4M dioxane) (5 mL, 20.0 mmol) at 0° C., and the mixture was stirred at 25° C. After 16 h, the reaction mixture was concentrated under reduced pressure to afford 2-amino-3-(oxazol-2-yl)-N—((R)-4-phenyl-1-((3aS, 4S, 6S, 7aR)-3a, 5, 5-trimethylhexahydro-4, 6-methanobenzo[d][1, 3, 2]dioxaborol-2-yl)butyl)propanamide hydrochloride (10-9, 500 mg), which was used in the next step without further purification. [M−H]⁻=464.

Synthesis of N-(3-(oxazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide [Step 7]: To a solution of pyrazine-2-carboxylic acid (10-10, 163 mg, 1.3 mmol) in THF (5 mL) was added IBCF (0.2 mL, 1.3 mmol) followed by NMM (0.2 mL, 1.3 mmol) at −15° C. After 45 min, a solution of 2-amino-3-(oxazol-2-yl)-N—((R)-4-phenyl-1-((3aS, 4S, 6S, 7aR)-3a, 5, 5-trimethylhexahydro-4, 6-methanobenzo[d][1, 3, 2]dioxaborol-2-yl)butyl) propanamide hydrochloride (10-9, 600 mg, 1.2 mmol) in DMF (1 mL) was added dropwise followed by NMM (0.2 mL, 1.2 mmol). After stirring for 1 h at the same temperature, the reaction was diluted with EtOAc and washed successively with 0.1 N aqueous HCl, 5% aqueous K₂CO₃, water and brine. The organic phase was dried over anhydrous Na₂SO₄, filtered and concentrated under reduced pressure to afford N-(3-(oxazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino) propan-2-yl)pyrazine-2-carboxamide (10-11, 400 mg). [M−H]⁻=570.

Synthesis of N-(3-(oxazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide (10-12) and N-(3-(oxazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide (10-13) [Step 8]: Two diastereomers of N-(3-(oxazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide (10-11, 400 mg) were separated by chiral HPLC (SFC) to afford N—((R)-3-(oxazol-2-yl)-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)propan-2-yl)pyrazine-2-carboxamide (10-12, 28 mg) and N—((S)-3-(oxazol-2-yl)-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)propan-2-yl)pyrazine-2-carboxamide (10-13, 25 mg). Absolute stereochemistry of the compounds was not determined.

10-12: [M−H]⁻=571.

10-13: [M−H]⁻=570.

Chiral HPLC (SFC) purification method: Prep SFC was performed on a C-Amylose A column (30 mm×250 mm), 5μ, using MeOH as the diluent with a flowrate of 60 g/min using mobile phase of 70% CO₂+30% of (0.3% TEA in MeOH) with ABPR of 100 bar at 35° C., and the fractions were detected using UV (274 nm).

Synthesis of ((R)-1-((R)-3-(oxazol-2-yl)-2-(pyrazine-2-carboxamido)propanamido)-4-phenyl butyl)boronic acid (Compound 10) [Step 9]: To an ice-cold solution of N—((R)-3-(oxazol-2-yl)-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)propan-2-yl)pyrazine-2-carboxamide (10-12, 28 mg, 0.05 mmol) and methylboronic acid (44 mg, 0.7 mmol) in acetone (1 mL) was added 0.2N HCl (1.0 mL, 0.2 mmol), and the mixture was stirred at 25° C. After 16 h, the reaction mixture was concentrated under reduced pressure, and then lyophilized. The material was purified by prep HPLC (RP) and lyophilized to afford ((R)-1-(3-(oxazol-2-yl)-2-(pyrazine-2-carboxamido)propanamido)-4-phenylbutyl)boronic acid (Compound 10, 18 mg). [M−H]⁻=436; ¹H NMR (400 MHz, MeOD): δ 9.22-9.21 (m, 1H), 8.80-8.79 (m, 1H), 8.69-8.68 (m, 1H), 7.80 (s, 1H), 7.23-7.19 (m, 2H), 7.15-7.10 (m, 3H), 7.05 (s, 1H), 5.33-5.30 (m, 1H), 3.49-3.46 (m, 2H), 2.69-2.66 (m, 1H), 2.61-2.56 (m, 2H), 1.63-1.62 (m, 2H), 1.61-1.43 (m, 2H). The absolute stereochemistry was not determined for this compound.

Synthesis of ((R)-1-((S)-3-(oxazol-2-yl)-2-(pyrazine-2-carboxamido)propanamido)-4-phenyl butyl)boronic acid (Compound 11) [Step 10]: To an ice-cold solution of N—((S)-3-(oxazol-2-yl)-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)propan-2-yl)pyrazine-2-carboxamide (10-13, 25 mg, 0.04 mmol) and methylboronic acid (39 mg, 0.6 mmol) in acetone (1 mL) was added 0.2N HCl (1.0 mL, 0.2 mmol), and the mixture was stirred at 25° C. After 16 h, the reaction mixture was concentrated under reduced pressure, and then lyophilized. The material was purified by prep HPLC (RP) and lyophilized to afford ((R)-1-((S)-3-(oxazol-2-yl)-2-(pyrazine-2-carboxamido)propanamido)-4-phenylbutyl)boronic acid (Compound 11, 13 mg). [M−H]⁻=436; ¹H NMR (400 MHz, MeOD): δ 9.21 (s, 1H), 8.80-8.79 (m, 1H), 8.69 (br s, 1H), 7.79 (s, 1H), 7.22-7.19 (m, 2H), 7.15-7.08 (m, 3H), 7.04 (s, 1H), 5.35-5.32 (m, 1H), 3.49-3.47 (m, 1H), 2.66-2.54 (m, 3H), 1.65-1.28 (m, 4H). The absolute stereochemistry was not determined for this compound.

### Example 12 and Example 13

### Synthesis of ((R)-1-((R)-3-(1-methyl-1H-Imidazol-2-yl)-2-(pyrazine-2-carboxamido) propanamido)-4-phenylbutyl)boronic acid (Compound 12) and ((R)-1-((S)-3-(1-methyl-1H-imidazol-2-yl)-2-(pyrazine-2-carboxamido)propanamido)-4-phenylbutyl)boronic acid (Compound 13)

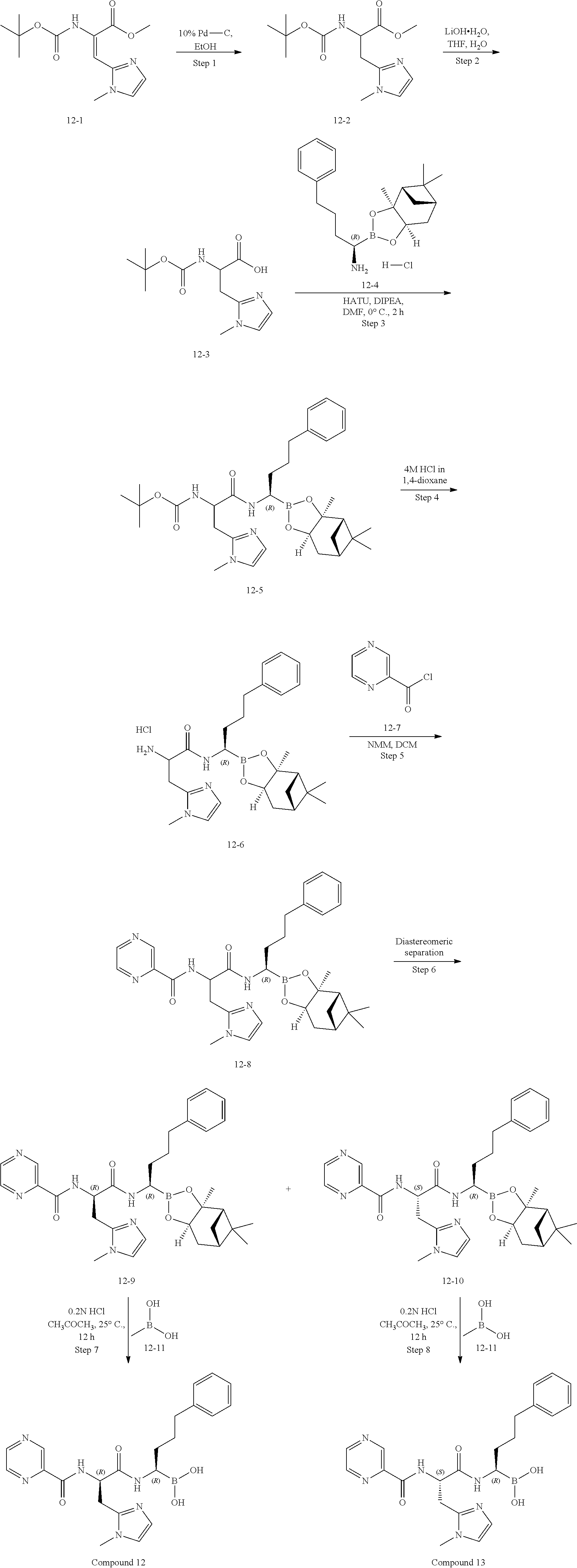

Synthesis of methyl 2-((tert-butoxycarbonyl)amino)-3-(1-methyl-1H-imidazol-2-yl)propanoate [Step 1]: To a stirred solution of methyl (E)-2-(tert-butoxycarbonylamino)-3-(3-methylimidazol-4-yl)prop-2-enoate (12-1, 1.05 g, 3.73 mmol) in Methanol (30 mL) was added 10% Pd—C(250 mg) and hydrogenated under H₂ balloon pressure at ambient temperature for 16 h. The reaction mixture was filtered through celite bed. Filtrate liquid was concentrated under reduced pressure to get methyl 2-(tert-butoxycarbonylamino)-3-(3-methylimidazol-4-yl) propanoate (12-2, 900 mg). [M+H]⁺=284; ¹H NMR (400 MHz, DMSO) δ 7.19 (d, 1H), 7.00 (s, 1H), 6.74 (s, 1H), 4.48-4.46 (m, 1H), 3.59 (s, 3H), 3.53 (s, 3H), 3.01-3.00 (m, 2H), 1.36 (s, 9H).

Synthesis of 2-((tert-butoxycarbonyl)amino)-3-(1-methyl-1H-Imidazol-2-yl)propanoic acid [Step 2]: To a stirred solution of methyl 2-(tert-butoxycarbonylamino)-3-(3-methylimidazol-4-yl)propanoate (12-2, 472 mg, 1.66 mmol) in THF (10 mL) was added a solution of LiOH·H2O (105 mg, 2.50 mmol) in water (2 mL) and stirred at ambient temperature for 16 h. The reaction mixture was concentrated and diluted with water. The aqueous part was washed with EtOAc. The residual aqueous part was acidified with 1N HCl and lyophilized to get 2-(tert-butoxycarbonylamino)-3-(3-methylimidazol-4-yl)propanoic acid (12-3, 380 mg). [M+H]⁺=270; ¹H NMR (400 MHz, DMSO-d₆): δ 7.04-6.99 (m, 2H), 6.79-6.76 (m, 1H), 4.35-4.33 (m, 1H), 3.59-3.58 (m, 1H), 3.55 (s, 3H), 3.01 (bs, 2H), 1.75 (bs, 1H), 1.35 (s, 9H).

Synthesis of tert-butyl (3-(1-methyl-1H-imidazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl) amino)propan-2-yl)carbamate [Step 3]: To a stirred solution of 2-((tert-butoxycarbonyl)amino)-3-(1-methyl-1H-imidazol-2-yl)propanoic acid (12-3, 163 mg, 0.6 mmol) in DMF (3 mL) was added HATU (250 mg, 0.65 mmol) followed by DIPEA (0.15 mL, 1.1 mmol) at 0° C. and stirred for 30 min. (R)-4-phenyl-1-((3aS, 4S, 6S, 7aR)-3a, 5, 5-trimethylhexahydro-4, 6-methanobenzo[d][1, 3, 2]dioxaborol-2-yl)butan-1-amine hydrochloride (12-4, 200 mg, 0.55 mmol) was added to it stirred at 0° C. for 2 h. It was quenched with 5% aqueous K₂CO₃ solution and extracted with EtOAc (thrice). Combined organic layer was washed with water and brine, dried over anhydrous Na₂SO₄ and evaporated under reduced pressure to get tert-butyl (3-(1-methyl-1H-imidazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)carbamate (12-5, 250 mg). [M−H]⁻=578.

Synthesis of 2-amino-3-(1-methyl-1H-imidazol-2-yl)-N—((R)-4-phenyl-1-((3aS, 4S, 6S, 7aR)-3a, 5, 5-trimethylhexahydro-4, 6-methanobenzo[d][1, 3, 2]dioxaborol-2-yl)butyl) propenamide hydrochloride [Step 4]: To a solution of tert-butyl (3-(1-methyl-1H-imidazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)carbamate (12-5, 250 mg, 0.4 mmol) in 1, 4-dioxane (3 mL) was added 4M HCl in 1, 4-dioxane (4 mL) at 0° C. It was gradually warmed to ambient temperature and stirred for 16 h. Volatiles were removed under reduced pressure to get 2-amino-3-(oxazol-5-yl)-N—((R)-4-phenyl-1-((3aS, 4S, 6S, 7aR)-3a, 5, 5-trimethylhexahydro-4, 6-methanobenzo[d][1, 3, 2]dioxaborol-2-yl)butyl)propanamide hydrochloride (12-6, 200 mg). [M−H]⁻=477.

Synthesis of N-(3-(1-methyl-1H-Imidazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino) propan-2-yl)pyrazine-2-carboxamide [Step 5]: To a stirred solution of NMM (0.05 mL, 0.38 mmol) in DCM (10 mL) was added 2-amino-3-(1-methylimidazol-2-yl)-N-[(1R)-4-phenyl-1-[(1S, 2S, 6R, 8S)-2, 9, 9-trimethyl-3, 5-dioxa-4-boratricyclo[6.1.1.02, 6]decan-4-yl]butyl]propanamide hydrochloride (12-6, 140 mg, 0.27 mmol) at 0-5° C. Pyrazine-2-carbonyl chloride (12-7, 140 mg, 0.27 mmol) was added to the reaction mixture. The reaction was stirred at ambient temperature for 1.5 h. Reaction mixture was diluted with water and extracted with (2×10 mL) DCM. Combined organic layer was washed with water and brine, dried over anhydrous Na₂SO₄ and evaporated under reduced pressure. The product was purified by prep HPLC purification and lyophilized to afford N-(3-(1-methyl-1H-imidazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide (12-8, 60 mg). [M−H]⁻=584.

Synthesis of chiral N-(3-(1-methyl-1H-imidazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide [Step 6]: N-(3-(1-methyl-1H-imidazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide (12-8, 60 mg) was purified via SFC chiral separation and lyophilized to afford the first product as N—((R)-3-(1-methyl-1H-imidazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl) pyrazine-2-carboxamide, Peak 1 (12-9, 18 mg) and the second product as N—((S)-3-(1-methyl-1H-imidazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide, Peak 2 (12-10, 17 mg). The absolute stereochemistry of these compounds was not determined.

12-9: N-(3-(1-methyl-1H-imidazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide, Peak 1: [M−H]⁻=584.

12-10: N-(3-(1-methyl-1H-imidazol-2-yl)-1-oxo-1-(((R)-4-phenyl-1-((3aS, 4S, 6S, 7aR)-3a, 5, 5-trimethylhexahydro-4, 6-methanobenzo[d][1, 3, 2]dioxaborol-2-yl)butyl)amino)propan-2-yl)pyrazine-2-carboxamide, Peak 2: [M−H]⁻=584.

SFC chiral HPLC method: Chiral Separation was performed on a Thar SFC-80 instrument. Column: Amylose A (30 mm×250), 5μ, operating at a temperature of 35° C. with a flow rate of: 60 ml/min, Mobile Phase: 60% CO₂+40% of (0.3% TEA in MeOH) held isocratic and isobaric up to 14 min with detection at a wavelength of 220 nm.

Synthesis of ((R)-1-((R)-3-(1-methyl-1H-Imidazol-2-yl)-2-(pyrazine-2-carboxamido) propanamido)-4-phenylbutyl)boronic acid (Compound 12) [Step 7]: To a stirred solution of N—((R)-3-(1-methyl-1H-imidazol-2-yl)-1-oxo-1-(((R)-4-phenyl-1-((3aS,4S,6S,7aR)-3a,5,5-trimethyl hexahydro-4,6-methanobenzo[d][1,3,2]dioxaborol-2-yl)butyl)amino)propan-2-yl)pyrazine-2-carboxamide (12-9, 25 mg, 0.04 mmol) and methylboronic acid (12-11, 25 mg, 0.4 mmol) in acetone (1.0 mL) was added 0.2 N_HCl (1.0 mL) and stirred at ambient temperature for 16 h. Volatiles were evaporated under reduced pressure and purified by prep HPLC purification and lyophilized to afford ((R)-1-((R)-3-(1-methyl-1H-imidazol-2-yl)-2-(pyrazine-2-carboxamido) propanamido)-4-phenylbutyl)boronic acid (Compound 12, 8 mg). [M−H]⁻=449; ¹H NMR (400 MHz, MeOD): δ 9.18 (s, 1H), 8.81 (d, 1H), 8.69 (d, 1H), 7.46-7.40 (m, 2H), 7.22-7.10 (m, 5H), 5.21 (s, 1H), 3.91 (s, 3H), 3.74-3.68 (m, 1H), 3.49-3.44 (m, 1H), 2.86-2.56 (m, 4H), 1.63-1.27 (m, 5H). The absolute stereochemistry of this compound was not determined.

Synthesis of ((R)-1-((S)-3-(1-methyl-1H-Imidazol-2-yl)-2-(pyrazine-2-carboxamido) propanamido)-4-phenylbutyl)boronic acid (Compound 13) [Step 8]: To a stirred solution of N-[(1S)-1-[(1-methylimidazol-2-yl)methyl]-2-oxo-2-[[(1R)-4-phenyl-1-[(1S,2S,6R,8S)-2,9,9-trimethyl-3,5-dioxa-4-boratricyclo[6.1.1.02,6]decan-4-yl]butyl]amino]ethyl]pyrazine-2-carboxamide (12-10, 25 mg, 0.0428 mmol) and methylboronic acid (12-11, 26 mg, 0.428 mmol) in acetone (1 mL) was added 0.2 N HCl (1.0 mL) and stirred at ambient temperature for 16 h. Volatiles were evaporated under reduced pressure and purified by prep HPLC purification and lyophilized to afford [(1R)-1-[[(2S)-3-(1-methylimidazol-2-yl)-2-(pyrazine-2-carbonylamino) propanoyl]amino]-4-phenyl-butyl]boronic acid (Compound 13, 8 mg). [M−H]⁻=449; ¹H NMR (400 MHz, MeOD): δ 9.17 (s, 1H), 8.81 (d, 1H), 8.70 (s, 1H), 7.44-7.40 (d, 2H), 7.22-7.19 (m, 2H), 7.14-7.11 (m, 3H), 5.27 (s, 1H), 3.90 (s, 3H), 3.71-3.69 (m, 1H), 3.48-3.44 (m, 1H), 2.81 (bs, 1H), 2.62-2.57 (m, 2H), 1.62-1.49 (m, 4H). The absolute stereochemistry of this compound was not determined.

### Example 14

### General Procedures for the Preparation of Compounds

The following general procedures are provided to prepare compounds that are prepared using similar reaction conditions.

General Procedure A: Amide Formation from an Amine and a Carboxylic Acid

To a stirred solution of a carboxylic acid containing compound in tetrahydrofuran (THF) was added isobutyl chloroformate (IBCF, 1 equivalent) and 4-methylmorpholine (NMM, 1 equivalent) at −15° C. The reaction mixture was stirred at the same temperature for about 30 minutes. The corresponding amine (0.9-1.1 equivalents) in dimethylformamide (DMF) was added followed by NMM (0.9-1.1 equivalents) at −15° C. The reaction mixture was gradually warmed to 0° C. and stirred for about 2 hours. The resulting product was neutralized with aqueous 0.1 N HCl solution and extracted several times with ethyl acetate. The organic layers were combined and washed with a solution of 5% potassium carbonate, water, brine, and dried over anhydrous sodium sulfate. The mixture was filtered, concentrated under reduced pressure, and purified through combiflash column chromatography to afford the corresponding amide product.

General Procedure B: Amide Formation from an Amine and an Acid Chloride, Anhydride, or Sulfonyl Chloride

To a stirred solution of the amine compound 0.6 mmol) and acetic anhydride (1.1 equivalents) in dichloromethane was added an ice cooled solution of diisopropylethylamine (DIPEA) (5 equivalents) and the reaction mixture was stirred at room temperature for about 2 hours. Thin layer chromatography showed complete disappearance of the starting material. The reaction mixture was diluted with dichloromethane (DCM) and washed with water and brine solution. The organic phase was dried over anhydrous sodium sulfate, filtered, and concentrated under reduced pressure. The product was purified by reverse-phase prep-HPLC and lyophilized to afford the desired amide product.

In General Procedure B, acetic anhydride can be replaced with an acyl chloride (e.g., morpholine-4-carbonyl chloride) or a sulfonyl chloride (e.g., benzenesulfonyl chloride), and the DIPEA can be replaced with another base (e.g., N-methylmorpholine, NMM).

### General Procedure C: Hydrogenolysis of Benzyl Esters

To a stirred solution of the benzyl ester containing compound in tetrahydrofuran (THF) was added nitrogen gas for 10 minutes. Then 10% Pd—C(0.7 equivalents) was added and the reaction mixture was hydrogenated under a hydrogen balloon from 3 to 12 hours and monitored by thin layer chromatography. The reaction mixture was filtered over celite using excess ethyl acetate. The solvents were removed by concentration under reduced pressure to afford the corresponding carboxylic acid product.

General Procedure D: Amide Formation with a Protected Boronic Acid

To a stirred solution of a carboxylic acid containing compound in tetrahydrofuran was added isobutyl chloroformate (IBCF) (1 equivalent) and N-methylmorpholine (NMM) (1 equivalent) at −15° C. The reaction mixture was stirred at the same temperature for about 30 minutes. Then the protected boronic acid compound bearing an amine group (1 equivalent) in dimethylformamide was added to the reaction mixture followed by NMM (1 equivalent) at −15° C. The reaction mixture was gradually warmed to 0° C. and stirred for about 2 hours. LCMS of the reaction mass confirmed the formation of the desired product, and the reaction mixture was neutralized with an aqueous solution of 0.1 N HCl and extracted with ethyl acetate. The organic layers were combined and washed with 5% potassium carbonate solution, water, brine, dried over sodium sulfate, filtered, and concentrated under reduced pressure to afford the coupled product.

### General Procedure E. Removal of a BOC Protection Group

To a solution of a BOC-protected compound was added 4 M HCl in dioxane (10 equivalents) at 0° C. The reaction mixture was gradually warmed to ambient temperature and stirred for about 16 hours. Thin layer chromatography showed complete consumption of the starting material and the reaction mixture was concentrated under reduced pressure to obtain the desired product as the hydrochloride salt. The product was used without purification.

### General Procedure F: Hydrolysis of a Boronate Ester

To a stirred solution of the boronate ester and methylboronic acid (8 equivalents) in acetone was added an equivalent volume of 0.2 N HCl and the reaction mixture was stirred at ambient temperature overnight. Thin layer chromatography showed the complete disappearance of the starting material and the reaction mixture was concentrated under reduced pressure. The product was redissolved in a mixture of acetone and deionized water, and lyophilized to obtain the boronic acid product.

### General Procedure G: Oxidative Removal of a Boronate Ester

To a stirred solution of a boronate ester in a mixture of (1:1) acetone and water was added ammonium acetate (1 equivalent) and the reaction mixture was stirred for 5 minutes. Sodium periodate (NaIO₄) (1 equivalent) was added portion-wise and reaction mixture was stirred for 3 hours. The reaction mixture was concentrated under reduced pressure and partitioned between ethyl acetate and water. The organic layer was collected and the aqueous layer was further extracted with ethyl acetate (twice). The combined organic layers were dried over anhydrous sodium sulfate and concentrated under reduced pressure. The product was purified through reverse-phase prep-HPLC to afford the desired boronic acid product.

### Example 15—Biological/Biochemical Evaluation

### General Protocol for In Vitro Analysis of Compounds

The inhibitory activity of the compounds of the present invention against LONP1, 20S proteasome and other proteases are determined by assays known to persons of ordinary skill in the art (see, e.g., Fishovitz, J. et al. “Active-Site-Directed Chemical Tools for Profiling Mitochondrial Lon Protease” ACS Chem. Biol. 6, 781-788 (2011)).

In this Example, LONP1 (NM_004793.4) activity was measured by a FRET-based assay for protease activity using a fluorogenic peptide DabcylYRGIT(2Abu)SGRQK(5-FAM) (Cambridge Research Biochemicals) as substrate. LONP1 activity is followed by an increase in fluorescence signal due to the degradation of the peptide. Inhibition of LONP1 protease activity by an inhibitor compound of the disclosure elicits a decrease in the fluorescent signal.

The assay is performed in a 384-well plate (Greiner, cat. #781076) using the following reagents and conditions: substrate (3 μM) was incubated for 1 hour at 37° C. in the presence of LONP1 (15 nM as monomer), 25 mM Tris pH 8.0, 10 mM MgCl₂, 0.03 mg/mL BSA, 0.5 mM DTT, 0.0003% Tween-20, 10 mM NaCl, 0.06 mM ATP and 0.5 mM EGTA in a 15 μL final volume. The LONP1-containing mix (10 μL) was incubated with the test compound for 15 min at 37° C. before adding the peptide-containing mix (5 μL). Solutions were dispensed using a small cassette-Multidrop Combi (Thermo Scientific). Fluorescence was measured using a PheraStar plate reader (BMG Labtech) FI-FRET EX 485 nm Em 520 nm.

The IC₅₀ values for binding to LONP1 are summarized in Table 2 below. Each value is based on an average of a minimum of two repeats.

TABLE 2
IC50 assay data for compounds of
the disclosure binding to LONP1;

| Compound No. | Activity IC₅₀ (μM) |
|---|---|
| 1 | A |
| 2 | A |
| 3 | A |
| 4 | A |
| 5 | A |
| 6 | A |
| 7 | B |
| 8 | B |
| 9 | B |
| 10 | A |
| 11 | A |
| 12 | A |
| 13 | B |

A: <0.05 μM;
B: 0.05-0.5 μM;
C: 0.5-5 μM;
D >5 μM

In one embodiment, beneficial compounds of this disclosure have IC₅₀ values of less than 5 μM. In another embodiment, beneficial compounds of this disclosure have IC₅₀ less than 2.5 μM. In another embodiment, beneficial compounds of this disclosure have IC₅₀ less than 1 μM. In another embodiment, beneficial compounds of this disclosure have IC₅₀ less than 0.5 μM. In another embodiment, beneficial compounds of this disclosure have IC₅₀ less than 0.1 μM. In another embodiment, beneficial compounds of this disclosure have IC₅₀ less than 0.05 μM. In another embodiment, beneficial compounds of this disclosure have IC₅₀ less than 0.01 μM.

### Cell Viability Assay:

### Materials and Kits:

Cell Proliferation Kit I (MTT), Merck, Cat #11465007001
DMEM GlutaMax, Thermo Fisher Scientific, Cat #31966021—for expansion and assay
DMEM GlutaMax, low glucose, Thermo Fisher Scientific, Cat #21885025—for expansion for cell viability assay
FBS, Gibco, Cat #A3840402

### Assay Procedure:

One day before treatment, 3,000-5,000/mL of 143b cells are placed in aliquots of 100 μL per well in flat bottom ThermoFisher 96 well plate. The starting seeding number is optimised in relation to the batch of cells and medium. The assay lasts for 8 days from seeding to MTT assay, and so the seeding number must be selected to avoid over-confluency at the last day of the assay.

On day 0, 100 μl of medium (Cat #21885025) is transferred to compound/DMSO plate, then the compound/DMSO-containing medium is transferred to the plates with pre-seeded cells.

Incubate for 7 days at 37° C., 5% CO₂ incubator.

On day 7, the medium is discarded. 100 μl of MTT labelling reagent mixed 1:10 in culture medium (Cat #21885025) is added and incubated for 4 hours at 37° C., 5% CO₂ incubator. 100 μl of MTT solubilization solution is added, mixed well and incubated overnight at 37° C.

Absorbance is measured at 570 nm on a plate reader.

### Compound Plate Setup:

The compounds are dispensed in a 96-well Greiner plate (cat no. 651201).
Volume of each compound solution: 200 nL
Final conc of DMSO: 0.1% in all wells
Starting concentration: 10 mM (final concentration on assay plate: 10 μM). In total, 8 doses and three replicates per dose per compound.
Dilution factor 3.162

The compounds are dissolved in DMSO and dispensed according to the concentration titrations and experimental design (indicated above).

Two plates of the same compounds are dispensed and the remaining plates are retained as a backup.

Compounds can be dispensed in an Echo dispenser and sealed immediately so that they are not exposed to air and contamination. The protocol is performed under the LAF bench.

On the first day of treatment (Day 0), compound plates are opened under the LAF bench. 100 μl of assay medium (Cat #21885025) is added to each well and 100.2 μl of medium+compound/DMSO is transferred to the assay plates containing pre-seeded cells.

1. A compound of structural Formula 1: 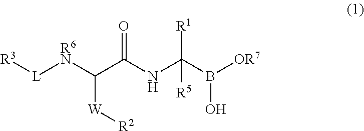
or a pharmaceutically acceptable salt, solvate, stereoisomer or mixture of stereoisomers, tautomer, isotopic form, pharmaceutically active metabolite thereof, or combinations thereof, wherein:
R¹ is selected from the group consisting of: deuterium, C1-C4 alkyl, C1-C4 alkoxyl, C₁-C4 oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R⁸, CONR⁸R⁹, NR⁸R⁹, SR⁸, SO2NR⁸R⁹, C1-C4 alkyl, C1-C4 haloalkyl, C1-C4 alkoxy, phenyl, or a 5- or 6-membered heteroaryl;
W is C1-C4 alkyl, optionally substituted with one or more of deuterium, halogen, hydroxyl, CN, methyl or ethyl;
R² is a 5 to 14 membered heterocyclic mono-, bi- or tricyclic ring optionally having one or more heteroatoms selected from N, O and S, wherein the heterocyclic ring is optionally substituted with one or more substituent selected from deuterium, halogen, hydroxyl, CN, C1-C4 alkyl, C1-C4 haloalkyl or C1-C4 alkoxy;
L is C(O), C(O)O, C(O)NR⁴, S(O)₂, or a bond;
R³ is C₁-C₄ alkyl optionally substituted with one or more substituents each independently selected from the group consisting of deuterium, halogen, cyano, hydroxyl, C₁-C₄ alkoxyl, 5 or 6 membered aryl (e.g. phenyl) or 5 or 6 membered heteroaryl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C₁-C₄ alkoxyl, or C₁-C₄ alkyl that is optionally substituted with one to three substituents selected from deuterium, halogen, cyano, hydroxyl, or C₁-C₄ alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R⁸, CONR⁸R⁹, NR⁸R⁹, SR⁸, SO2NR⁸R⁹, C₁-C₄ alkoxyl, or C₁-C₄ alkyl that is optionally substituted with one to three substituents selected from deuterium, halogen, cyano, hydroxyl, or C₁-C₄ alkoxyl;
R⁴ is hydrogen, deuterium, or C1-C4 alkyl optionally substituted with one or more of halogen, hydroxyl and phenyl, wherein phenyl is optionally substituted with one or more substituent selected from halogen, hydroxyl and C1-C2 alkyl;
R⁵ is selected from hydrogen, deuterium or C1-C2 alkyl;
R⁶ is selected from hydrogen, deuterium or C1-C2 alkyl optionally substituted with one or more substituents each independently selected from the group consisting of halogen, hydroxyl, cyano, methoxyl and phenyl;
R⁷ is hydrogen, or R⁷ and R¹, together with the boron atom to which —OR⁷ is attached form a 5-membered heteroalkyl ring; and
R⁸ and R⁹ are each independently selected from hydrogen, deuterium, C1-C4 alkyl; C1-C4 haloalkyl, C1-C5 alkyl-alkoxyl, C3-C7 cycloalkyl, or R⁸ and R⁹ together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

2. A compound according to claim 1, wherein R¹ is selected from methyl, ethyl, n-propyl, i-propyl, n-butyl or tert-butyl, each optionally substituted with a phenyl ring.

3. The compound according to claim 1, wherein R¹ is selected from methyl, n-propyl, n-butyl or tert-butyl.

4. The compound according to claim 1, wherein R¹ is selected from phenyl-(CH₂)₂— or phenyl-(CH₂)₃—.

5. The compound according to claim 1, wherein R¹ is selected from tert-butyl or phenyl-(CH₂)₃—.

6. The compound according to claim 1, wherein W is C1-C2 alkyl, optionally substituted with one or more of deuterium, halogen, hydroxyl, CN, methyl or ethyl.

7. The compound according to claim 1, wherein W is selected from methyl or ethyl, wherein the methyl or ethyl is optionally substituted with one to three substituents selected from deuterium, F, Cl, hydroxyl or methyl.

8. The compound according to claim 1, wherein W is methyl or ethyl.

9. The compound according to claim 1, wherein W is methyl.

10. The compound according to claim 1, wherein R² is a 5 or 6 membered heterocyclic ring having one or more heteroatoms selected from N, O and S, wherein the heterocyclic ring is optionally substituted with one or more substituent selected from deuterium, halogen, hydroxyl, CN, C1-C4 alkyl, C1-C4 haloalkyl or C1-C4 alkoxy.

11. The compound according to claim 1, wherein R² is a 5 or 6 membered heterocyclic ring having one or two heteroatoms selected from N, O and S, wherein the heterocyclic ring is optionally substituted with one or more substituent selected from deuterium, halogen, hydroxyl, CN, methyl, ethyl, C1-C2 haloalkyl or C1-C2 alkoxy.

12. The compound according to claim 1, wherein R² is a 5 or 6 membered heterocyclic ring having one heteroatom selected from N, O and S, wherein the heterocyclic ring is optionally substituted with one or more substituent selected from F, Cl, hydroxyl, methyl, ethyl, wherein the methyl and ethyl are optionally substituted with one or more halogen or deuterium.

13. The compound according to claim 1, wherein R² is a 5 or 6 membered heterocyclic ring having two heteroatoms selected from N, O and S, wherein the heterocyclic ring is optionally substituted with one or more substituent selected from F, Cl, hydroxyl, methyl, ethyl, wherein the methyl and ethyl are optionally substituted with one or more halogen or deuterium.

14. The compound according to claim 1, wherein R² is a 9 or 10 membered bicyclic heterocyclic ring having one or more heteroatoms selected from N, O and S, wherein the heterocyclic ring is optionally substituted with one or more substituent selected from deuterium, halogen, hydroxyl, CN, C1-C4 alkyl, C1-C4 haloalkyl or C1-C4 alkoxy.

15. The compound according to claim 1, wherein R² is a 9 or 10 membered heterocyclic ring having one or two heteroatoms selected from N, O and S, wherein the heterocyclic ring is optionally substituted with one or more substituent selected from deuterium, halogen, hydroxyl, CN, methyl, ethyl, C1-C2 haloalkyl or C1-C2 alkoxy.

16. The compound according to claim 1, wherein R² is a 9 or 10 membered heterocyclic ring having one heteroatom selected from N, O and S, wherein the heterocyclic ring is optionally substituted with one or more substituent selected from F, Cl, hydroxyl, methyl, ethyl, wherein the methyl and ethyl are optionally substituted with one or more halogen or deuterium.

17. The compound according to claim 1, wherein R² is a 9 or 10 membered heterocyclic ring having two heteroatoms selected from N, O and S, wherein the heterocyclic ring is optionally substituted with one or more substituent selected from F, Cl, hydroxyl, methyl, ethyl, wherein the methyl and ethyl are optionally substituted with one or more halogen or deuterium.

18. The compound according to claim 10, wherein the heteroatom or heteroatoms are selected from: (i) N; (ii) N and O; (iii) N and S; or (iv) O and S.

19. The compound according to claim 1, wherein R² is a heterocyclic ring selected from optionally substituted: tetrahydrofuranyl, furanyl, pyrrolidinyl, pyrrolyl, thiophenyl, imidazolyl, pyrazolyl, oxazolyl, isooxazolyl, thiazolyl, isothiazolyl, oxadiazolyl, pyridinyl, piperidinyl, pyridazinyl, piperazinyl, pyrimidinyl, pyrazinyl, tetrahydropyranyl, pyranyl, dioxanyl, morpholinyl, azepanyl, oxepanyl, oxazepanyl, pyrrolizidinyl, indolyl, isoindolyl, indolizinyl, benzimidazolyl, purinyl, quinolinyl, isoquinolinyl, quinazolinyl, or pteridinyl; and wherein the heterocyclic ring is joined to W via a carbon atom or via a heteroatom.

20. The compound according to claim 1, wherein R² is a heterocyclic ring selected from: 2-, 3-, 4- or 5-tetrahydrofuranyl, 2-, 3-, 4- or 5-furanyl, 1-, 2, 3-, 4- or 5-pyrrolidinyl, 1-, 2-, 3-, 4- or 5-pyrrolyl, 2-, 3-, 4- or 5-thiophenyl, 1-, 2-, 3-, 4- or 5-imidazolyl, 1-, 2-, 3-, 4- or 5-pyrazolyl, 2-, 3-, 4- or 5-oxazolyl, 2-, 3-, 4- or 5-isooxazolyl, 2, 3-, 4- or 5-thiazolyl, 2-, 3-, 4- or 5-isothiazolyl, 2-, 3-, 4- or 5-oxadiazolyl, 1-, 2-, 3-, 4-, 5- or 6-pyridinyl, 1-, 2-, 3-, 4-, 5- or 6-piperidinyl, 1-, 2-, 3-, 4-, 5- or 6-pyridazinyl, 1-, 2-, 3-, 4-, 5- or 6-piperazinyl, 1-, 2-, 3-, 4-, 5- or 6-pyrimidinyl, 1-, 2-, 3-, 4-, 5- or 6-pyrazinyl, 1-, 2-, 3-, 4-, 5- or 6-tetrahydropyranyl, 2-, 3-, 4-, 5- or 6-pyranyl, 2-, 3-, 5- or 6-dioxanyl, 2-, 3-, 4-, 5- or 6-morpholinyl, 1-, 2-, 3-, 4-, 5-, 6- or 7-azepanyl, 2-, 3-, 4-, 5-, 6- or 7-oxepanyl, 2-, 3-, 4-, 5-, 6- or 7-oxazepanyl, 1-, 2-, 3-, 5-, 6-, 7- or 8-pyrrolizidinyl, 1-, 2-, 3-, 4-, 5-, 6- or 7-indolyl, 1-, 2, 3-, 4-, 5-, 6-, 7-isoindolyl, 1-, 2-, 3-, 5-, 6-, 7- or 8-indolizinyl, 1-, 2-, 3-, 4-, 5-, 6- or 7-benzimidazolyl, 1-, 2-, 3-, 6-, 7-, 8- or 9-purinyl, 1-, 2-, 3-, 4-, 5-, 6-, 7- or 8-quinolinyl, 1-, 2, 3-, 4-, 5-, 6-, 7- or 8-isoquinolinyl, 1-, 2-, 3-, 4-, 5-, 6-, 7- or 8-quinazolinyl, or 1-, 2-, 3-, 4-, 5-, 6-, 7- or 8-pteridinyl

21. The compound according to claim 1, wherein R² is a heterocyclic ring selected from: 2, 3- or 5-tetrahydrofuranyl, 2, 3- or 5-furanyl, 2, 3- or 5-pyrrolidinyl, 2, 3- or 5-pyrrolyl, 2, 3- or 5-thiophenyl, 2, 3- or 5-imidazolyl, 2, 3- or 5-pyrazolyl, 2, 3- or 5-oxazolyl, 2, 3- or 5-isooxazolyl, 2, 3- or 5-thiazolyl, 2, 3- or 5-isothiazolyl, 2, 3- or 5-oxadiazolyl, 2, 3- or 4-pyridinyl, 2, 3- or 4-piperidinyl, 2, 3- or 4-pyridazinyl, 2, 3- or 4-piperazinyl, 2, 3- or 4-pyrimidinyl, 2, 3- or 4-pyrazinyl, 2, 3- or 4-tetrahydropyranyl, 2, 3- or 4-pyranyl, 2- or 3-dioxanyl, 2, 3- or 4-morpholinyl, 2, 3- or 4-azepanyl, 2, 3- or 4-oxepanyl, 2, 3- or 4-oxazepanyl, 2, 3- or 5-pyrrolizidinyl, 2, 3- or 4-indolyl, 2, 3- or 4-isoindolyl, 2, 3- or 5-indolizinyl, 2, 3- or 4-benzimidazolyl, 2, 3- or 6-purinyl, 2, 3- or 4-quinolinyl, 2, 3- or 4-isoquinolinyl, 2, 3- or 4-quinazolinyl, or 2, 3- or 4-pteridinyl.

22. The compound according to claim 1, wherein R² is a heterocyclic ring selected from: imidazolyl, pyrazolyl, oxazolyl, thiazolyl or indolyl.

23. The compound according to claim 1, wherein R² is a heterocyclic ring selected from: 2-imidazolyl, 3-pyrazolyl, 2-oxazolyl, 5-oxazolyl, 2-thiazolyl or 3-indolyl.

24. The compound according to claim 10, wherein R² is substituted with methyl, and wherein methyl may be attached to a carbon atom or to a heteroatom.

25. The compound according to claim 1, wherein R² is a heterocyclic ring selected from: 1-methyl-2-imidazolyl, 1-methyl-3-pyrazolyl, 2-oxazolyl, 5-oxazolyl, 2-thiazolyl or 3-indolyl.

26. The compound according to claim 1, wherein R² is a heterocyclic ring selected from: 1-methyl-2-imidazolyl, 1-methyl-3-pyrazolyl.

27. The compound according to claim 1, having the structural formula 2: 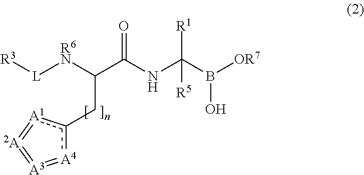
wherein:
n is 1, 2 or 3;
each of A¹ to A⁴ are independently selected from C(R⁸), N(R⁹), N, O or S;
R⁸ is selected from hydrogen, deuterium, halogen, hydroxyl, CN, C1-C4 alkyl, C1-C4 haloalkyl or C1-C4 alkoxy; and
R⁹ is selected from hydrogen, deuterium, C1-C4 alkyl or C1-C4 haloalkyl.

28. The compound according to claim 27, wherein one, two or three of A¹ to A⁴ are C(R⁸).

29. The compound according to claim 27, wherein two of A¹ to A⁴ are C(R⁸).

30. The compound according to claim 27, wherein three of A¹ to A⁴ are C(R⁸).

31. The compound according to claim 27, wherein A¹ is selected from N(R⁹), N, O or S.

32. The compound according to claim 27, wherein A² is selected from N(R⁹), N, O or S.

33. The compound according to claim 27, wherein A³ is selected from N(R⁹), N, O or S.

34. The compound according to claim 27, wherein A⁴ is selected from N(R⁹), N, O or S.

35. The compound according to claim 27, wherein A¹ is selected from N(R⁹) or N; and A² is selected from N(R⁹) or N.

36. The compound according to claim 27, wherein A¹ is selected from N(R⁹) or N; and A⁴ is selected from N(R⁹) or N.

37. The compound according to claim 27, wherein A¹ is selected from N(R⁹) or N; and A⁴ is selected from O or S.

38. The compound according to claim 27, wherein A² is selected from N(R⁹) or N; and A⁴ is selected from O or S.

39. The compound according to claim 27, wherein A² is C(R⁸); and A³ is C(R⁸).

40. The compound according to claim 27, wherein R⁸ is selected from hydrogen, F, Cl, hydroxyl, methyl, ethyl, CF₃, or OMe.

41. The compound according to claim 27, wherein R⁹ is selected from hydrogen or methyl.

42. The compound according to claim 27, wherein R⁹ is selected from hydrogen, methyl, ethyl or OMe.

43. The compound according to claim 27, wherein R⁹ is selected from hydrogen or methyl.

44. The compound according to claim 27, wherein n is 1 or 2.

45. The compound according to claim 27, wherein n is 1.

46. The compound according to claim 1, wherein L is selected from C(O), C(O)O, C(O)NH, C(O)N(CH₃), SO₂.

47. The compound according to claim 1, wherein L is selected from C(O), C(O)O and C(O)NH.

48. The compound according to claim 1, wherein L is C(O).

49. The compound according to claim 1, wherein R³ is C₁-C₄ alkyl, a 5- or 6-membered heteroaryl, C6 aryl, a 5- or 6-membered heterocycloalkyl or C6 cycloalkyl, and wherein R³ is optionally substituted.

50. The compound according to claim 1, wherein R³ is methyl, ethyl, n-propyl, i-propyl, n-butyl or tert-butyl, each optionally substituted with a phenyl ring.

51. The compound according to claim 50, wherein R³ is selected from methyl, i-propyl and tert-butyl.

52. The compound according to claim 1, wherein R³ is selected from phenyl, phenyl-(CH₂)— and phenyl-(CH₂)₂—, wherein the phenyl group is optionally substituted.

53. The compound according to claim 1, wherein R³ is selected from an aryl, heteroaryl, cycloalkyl or heterocycloalkyl selected from tetrahydropyranyl, pyrazinyl, tetrahydropyrrolyl, tetrahydrofuranyl, tetrahydropyranyl, cyclohexanyl, oxazolyl and morpholinyl, wherein said aryl, heteroaryl, cycloalkyl or heterocycloalkyl is optionally substituted.

54. The compound according to claim 53, wherein R³ is selected from n-tetrahydropyrrolyl, morpholinyl and pyrazinyl.

55. The compound according to claim 52, wherein said substituent is selected from one to three of halogen, hydroxyl and C1-C2 alkyl.

56. The compound according to claim 52, wherein said substituent is selected from one or two of Cl, hydroxyl and methyl.

57. The compound according to claim 52, wherein R³ is selected from 2-chlorophenyl, 3-chlorophenyl, 2,5-dichlorophenyl, 2,4-dimethyloxazolyl, and 3-hydroxy n-tetrahydropyrrolyl.

58. The compound according to claim 1, wherein R⁴ is hydrogen or methyl.

59. The compound according to claim 1, wherein R⁵ is hydrogen or C1-C2 alkyl.

60. The compound according to claim 1, wherein R⁵ is hydrogen.

61. The compound according to claim 1, wherein R⁶ is selected from hydrogen, phenyl-(CH₂)— or phenyl-(CH₂)₂—.

62. The compound according to claim 1, wherein R⁶ is hydrogen.

63. The compound according to claim 1, wherein R⁷ is hydrogen.

64. The compound according to claim 1, wherein R⁸ and R⁹ are each independently selected from hydrogen, deuterium, C1-C2 alkyl; C1-C2 haloalkyl, C1-C2 alkyl-alkoxyl or C3-C7 cycloalkyl, wherein C3-C7 cycloalkyl is optionally substituted with one or more substituent selected from deuterium, F, Cl, hydroxyl, oxo, CN, C1-C2 alkyl, C1-C2 haloalkyl or C1-C2 alkoxyl.

65. The compound according to claim 1, wherein R⁸ and R⁹ together with the N to which they are attached form 3 to 7 membered heterocyclic ring optionally having one or two additional heteroatoms selected from N, O and S, which is optionally substituted with one or more substituent selected from deuterium, F, Cl, hydroxyl, oxo, CN, C1-C2 alkyl, C1-C2 haloalkyl or C1-C2 alkoxyl.

66. The compound according to claim 1, wherein halogen is selected from fluoro or chloro.

67. The compound according to claim 1, wherein halogen is chloro.

68. The compound according to claim 1, which is selected from any one of:
(R)-4-phenyl-1-((R)-2-(pyrazine-2-carboxamido)-3-(thiazol-2-yl)propanamido)butyl) boronic acid;
((R)-4-phenyl-1-((S)-2-(pyrazine-2-carboxamido)-3-(thiazol-2-yl)propanamido)butyl) boronic acid;
((R)-1-((R)-3-(1-methyl-1H-pyrazol-3-yl)-2-(pyrazine-2-carboxamido)propanamido)-4-phenylbutyl) boronic acid;
((S)-1-((R)-3-(1-methyl-1H-pyrazol-3-yl)-2-(pyrazine-2-carboxamido)propanamido)-4-phenylbutyl) boronic acid;
((R)-1-((R)-3-(oxazol-5-yl)-2-(pyrazine-2-carboxamido)propanamido)-4-phenylbutyl) boronic acid;
(R)-1-((S)-3-(oxazol-5-yl)-2-(pyrazine-2-carboxamido)propanamido)-4-phenylbutyl) boronic acid;
((R)-1-((R)-3-(1H-indol-3-yl)-2-(pyrazine-2-carboxamido)propanamido)-4-phenylbutyl) boronic acid;
((R)-3-methyl-1-((R)-3-(1-methyl-1H-imidazol-2-yl)-2-(pyrazine-2-carboxamido) propanamido)butyl) boronic acid;
((R)-3-methyl-1-((S)-3-(1-methyl-1H-imidazol-2-yl)-2-(pyrazine-2-carboxamido) propanamido)butyl) boronic acid;
((R)-1-((R)-3-(oxazol-2-yl)-2-(pyrazine-2-carboxamido)propanamido)-4-phenylbutyl) boronic acid;
((R)-1-((S)-3-(oxazol-2-yl)-2-(pyrazine-2-carboxamido)propanamido)-4-phenylbutyl) boronic acid;
((R)-1-((R)-3-(1-methyl-1H-imidazol-2-yl)-2-(pyrazine-2-carboxamido)propanamido)-4-phenylbutyl) boronic acid; and
((R)-1-((S)-3-(1-methyl-1H-imidazol-2-yl)-2-(pyrazine-2-carboxamido)propanamido)-4-phenylbutyl) boronic acid.

69. The compound according to claim 1, which is selected from any one of structures 1 to 13.

70. The compound according to claim 1, which is selected from a compound of the group consisting of:
(i) compound 1, 2, 3, 4, 5, 6, 10, 11 and 12;
(ii) compound 7, 8, 9 and 13; or
(iii) structure 1, 3, 4, 5, 6, 10 and 11.

71. The compound according to claim 1, wherein the compound is an inhibitor of LONP1.

72. A pharmaceutical composition comprising one or more compounds according to claim 1 or pharmaceutically acceptable salt, solvate, stereoisomer or mixture of stereoisomers, tautomer, isotopic form, or pharmaceutically active metabolite thereof, or combinations thereof, and one or more pharmaceutically acceptable carrier.

73. A pharmaceutical composition comprising a compound according to Formula 1, 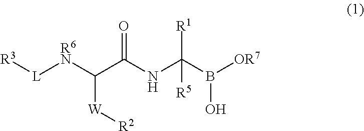
or a pharmaceutically acceptable salt, solvate, stereoisomer or mixture of stereoisomers, tautomer, isotopic form, or pharmaceutically active metabolite thereof, or combinations thereof, and one or more pharmaceutically acceptable carrier, wherein:
R¹ is selected from the group consisting of: deuterium, C1-C4 alkyl, C1-C4 alkoxyl, C₁-C₄ oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R⁸, CONR⁸R⁹, NR⁸R⁹, SR⁸, SO2NR⁸R⁹, C1-C4 alkyl, C1-C4 haloalkyl, C1-C4 alkoxy, phenyl, or a 5- or 6-membered heteroaryl;
W is C1-C4 alkyl, optionally substituted with one or more of deuterium, halogen, hydroxyl, CN, methyl or ethyl;
R² is a 5 to 14 membered heterocyclic mono-, bi- or tricyclic ring optionally having one or more heteroatoms selected from N, O and S, wherein the heterocyclic ring is optionally substituted with one or more substituent selected from deuterium, halogen, hydroxyl, CN, C1-C4 alkyl, C1-C4 haloalkyl or C1-C4 alkoxy;
L is C(O), C(O)O, C(O)NR⁴, S(O)₂, or a bond;
R³ is C₁-C₄ alkyl optionally substituted with one or more substituents each independently selected from the group consisting of deuterium, halogen, cyano, hydroxyl, C₁-C₄ alkoxyl, 5 or 6 membered aryl (e.g. phenyl) or 5 or 6 membered heteroaryl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C₁-C₄ alkoxyl, or C₁-C₄ alkyl that is optionally substituted with one to three substituents selected from deuterium, halogen, cyano, hydroxyl, or C₁-C₄ alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R⁸, CONR⁸R⁹, NR⁸R⁹, SR⁸, SO2NR⁸R⁹, C₁-C₄ alkoxyl, or C₁-C₄ alkyl that is optionally substituted with one to three substituents selected from deuterium, halogen, cyano, hydroxyl, or C₁-C₄ alkoxyl;
R⁴ is hydrogen, deuterium, or C1-C4 alkyl optionally substituted with one or more of halogen, hydroxyl and phenyl, wherein phenyl is optionally substituted with one or more substituent selected from halogen, hydroxyl and C1-C2 alkyl;
R⁵ is selected from hydrogen, deuterium or C1-C2 alkyl;
R⁶ is selected from hydrogen, deuterium or C1-C2 alkyl optionally substituted with one or more substituents each independently selected from the group consisting of halogen, hydroxyl, cyano, methoxyl and phenyl;
R⁷ is hydrogen, or R⁷ and R¹, together with the boron atom to which —OR⁷ is attached form a 5-membered heteroalkyl ring; and
R⁸ and R⁹ are each independently selected from hydrogen, deuterium, C1-C4 alkyl; C1-C4 haloalkyl, C1-C5 alkyl-alkoxyl, C3-C7 cycloalkyl, or R⁸ and R⁹ together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

74. The pharmaceutical composition of claim 73, wherein the compound of Formula 1 is defined according to claim 2.

75. The compound according to claim 1 for use in the treatment of a disease or disorder.

76. The compound for use according to claim 75, wherein the disease or disorder is characterized by mitochondrial dysfunction, such as mitochondrial disorders, including a neurodegenerative disorder, a metabolic disorder and a disease associated with the aging process.

77. The compound for use according to claim 75, wherein the disease or disorder is an oncologic disease or disorder, such as a cancer and/or a proliferative disease or disorder.

78. The compound for use according to claim 77, wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma colon cancer, colorectal cancer, cancer of connective tissue, esophageal cancer, embryonal carcinoma, fibrosarcoma, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas, mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML).

79. The compound for use according to claim 75, wherein the use comprises administering the compound orally; topically; by inhalation; by intranasal administration; by intracerebroventricular; or systemically by intravenous, intraperitoneal, subcutaneous, or intramuscular injection.

80. The compound for use according to claim 75, wherein the use comprises administering to a subject one or more compounds according to claim 1, optionally in combination with one or more additional therapeutic agent.

81. The compound for use according to claim 80, wherein the administering comprises administering the one or more compounds according to claim 1 simultaneously, sequentially or separately from the one or more additional therapeutic agent.

82. A method for treating or preventing a disease or disorder in a subject where inhibition of LONP1 may be beneficial, wherein said method comprises administering to the subject one or more compounds according to claim 1.

83. The method according to claim 82, wherein the disease or disorder is characterized by mitochondrial dysfunction, such as mitochondrial disorders, including a neurodegenerative disorder, a metabolic disorder and a disease associated with the aging process.

84. The method according to claim 82, wherein the disease or disorder is an oncologic disease or disorder, such as a cancer and/or a proliferative disease or disorder.

85. The method according to claim 84, wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma colon cancer, colorectal cancer, cancer of connective tissue, esophageal cancer, embryonal carcinoma, fibrosarcoma, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas, mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML).

86. The method according to claim 82, wherein one or more compounds according to claim 1 is administered in combination with one or more additional therapeutic agent.

87. The method according to claim 86, wherein the administering comprises administering the one or more compounds according to claim 1 simultaneously, sequentially or separately from the one or more additional therapeutic agent.

88. The method according to claim 82, wherein the method comprises administering the compound orally; topically; by inhalation; by intranasal administration; by intracerebroventricular; or systemically by intravenous, intraperitoneal, subcutaneous, or intramuscular injection.
